SEQUENCE LISTING (10 sequences)

SEQ ID NO: 1 - DNA, 23 bp, Artificial Sequence
  Description of Artificial Sequence Synthetic primer
ggctactgctgactctcaacatt

SEQ ID NO: 2 - DNA, 19 bp, Artificial Sequence
  Description of Artificial Sequence Synthetic primer
tcatggtggcgaattcgat

SEQ ID NO: 3 - DNA, 20 bp, Artificial Sequence
  Description of Artificial Sequence Synthetic primer
caaatagccctggcagattc

SEQ ID NO: 4 - DNA, 20 bp, Artificial Sequence
  Description of Artificial Sequence Synthetic primer
tgatacaagggacatcttcc

SEQ ID NO: 5 - DNA, 16 bp, Artificial Sequence
  Description of Artificial Sequence Synthetic primer
ttcatctgcaccaccg

SEQ ID NO: 6 - DNA, 17 bp, Artificial Sequence
  Description of Artificial Sequence Synthetic primer
ttgaagaagatggtgcg

SEQ ID NO: 7 - protein, 4 aa, Artificial Sequence
  Description of Artificial Sequence Synthetic peptide
RGDS

SEQ ID NO: 8 - DNA, 22 bp, Artificial Sequence
  Description of Artificial Sequence Synthetic primer
cacaaaaacaggttaaacccag

SEQ ID NO: 9 - DNA, 20 bp, Artificial Sequence
  Description of Artificial Sequence Synthetic primer
agcacataggaggcagagac

SEQ ID NO: 10 - DNA, 20 bp, Artificial Sequence
  Description of Artificial Sequence Synthetic primer
gaagacagaaaaggggaggg

(19) United States
(12) Patent Application Publication
(10) Pub. No.: US 2020/0132691 A1
(43) Pub. Date: Apr. 30, 2020

(54) DETECTION AND TARGETING OF TUMOR-PROMOTING NEUTROPHILS

(71) Applicants: THE GENERAL HOSPITAL CORPORATION, Boston, MA (US); PRESIDENT AND FELLOWS OF HARVARD COLLEGE, Cambridge, MA (US)

(72) Inventors: Mikael PITTET, Charlestown, MA (US); Camilla ENGBLOM, Boston, MA (US); Christina PFIRSCHKE, Boston, MA (US); Allon M KLEIN, Brookline, MA (US)

(73) Assignees: THE GENERAL HOSPITAL CORPORATION, Boston, MA (US); PRESIDENT AND FELLOWS OF HARVARD COLLEGE, Cambridge, MA (US)

(21) Appl. No.: 16/607,662

(22) PCT Filed: Apr. 24, 2018
(86) PCT No.: PCT/US2018/029072

§ 371 Date: Oct. 23, 2019

Related U.S. Application Data
(60) Provisional application No. 62/489,118, filed on Apr. 24, 2017.
(60) Provisional application No. 62/592,048, filed on Nov. 29, 2017.

(51) Int. Cl. G01N 33/574

(52) U.S. Cl. CPC G01N 33/57423

(57) ABSTRACT

Described herein are methods and compositions for treating cancer. Aspects of the invention relate to administering to a subject an agent that inhibits the activity, level, and/or migration of a SiglecFhigh cell. Another aspect of the invention relates to first identifying a population of SiglecFhigh cells in a patient and then administering an agent to the patient that inhibits the activity, level, Sand/or migration of said population of SiglecFhigh cells. In one embodiment, the patient has lung cancer.

### CROSS-REFERENCE TO RELATED APPLICATIONS

This application claims benefit under 35 U.S.C. § 119(e) of U.S. Provisional Application Nos. 62/489,118 filed Apr. 24, 2017 and 62/592,048 filed Nov. 29, 2017, the contents of which are incorporated herein by reference in their entireties.

### TECHNICAL FIELD

The field of the invention relates to the treatment of cancer.

### BACKGROUND

Myeloid cells have emerged as key regulators of cancer growth due to their abundance in the tumor stroma in a broad range of cancers, association with patient disease outcome and ability to modulate tumor progression (1-4). Most tumor-infiltrating myeloid cells are continuously replenished by circulating precursors, which are produced in distant tissues (4, 5) and some tumors amplify myeloid cell activity by skewing hematopoiesis toward the myeloid lineage or increasing myeloid cell populations in the periphery (6-8). For example, patients across cancer types present with elevated levels of hematopoietic myeloid progenitor cells in peripheral blood (9). Additionally, increased numbers of circulating myeloid cells, e.g., neutrophils, often correlate with poorer clinical outcome (10-12). It is therefore important to consider host changes that occur away from the tumor stroma to more fully understand the biological processes underlying tumor growth.

The bone marrow is a tissue of particular interest as it is the main site of hematopoietic cell production for all circulating blood lineages in the adult (13). The marrow contains resident cell components that not only participate in bone maintenance but also regulate hematopoiesis and immune cell fate, at least at steady-state (14-16). For example, osteoblasts, which are bone-forming cells, were the first bone-resident cells identified to regulate hematopoiesis (13, 14, 17). However, an understanding of bone dynamics in the context of cancer (at sites distant from the local bone microenvironment) and related immune responses remains limited. To address this knowledge gap, it should be determined whether a common solid cancer, e.g., lung adenocarcinoma, affects bone tissue, and how this might shape tumor-associated hematopoietic responses and distant tumor growth.

### SUMMARY

Presented herein are data that show solid tumor cancers, specifically lung cancer, induce the production of a osteocalcin-expressing (Ocn⁺) osteoblastic cells, which expand a distinct subset of tumor-infiltrating neutrophils defined as SiglecF^{high} (thereafter, referred to as SiglecF^{high} neutrophils). Findings presented herein show that SiglecF^{high} neutrophils exhibit tumor-promoting functions and promote tumor progression. Elimination of the SiglecF^{high} neutrophil population can reduce the capacity for a tumor microenvironment to protect that tumor, thus targeting these neutrophils can be an effective anti-cancer strategy. Presented herein is the SiglecF^{high} neutrophils gene profile, which is useful in targeting said SiglecF^{high} neutrophil population, for example, for cell death. Accordingly, one aspect of the invention described herein provides a method for treating cancer, the method comprising, administering an agent that inhibits the activity, level, and/or migration of a SiglecF^{high} cell.

Another aspect of the invention described herein provides a method for treating cancer, the method comprising identifying a population of SiglecF^{high} cells in a patient and administering an agent that inhibits the activity, level, and/or migration of said population of SiglecF^{high} cells.

Yet another aspect of the invention described herein provides a method of treating non-small cell lung cancer, the method comprising administering an agent that inhibits the activity, level, and/or migration of a SiglecF^{high} cell. In one embodiment, the method further comprises, before administering said agent, identifying in a patient a population of SiglecF^{high} cells.

In one embodiment of various aspects, the cancer is lung cancer, non-small cell lung cancer, KRAS⁺ non-small cell lung cancer, small cell lung cancer, small cell carcinoma, combined small cell carcinoma, lung carcinoid tumor, adenocarcinoma, squamous cell carcinoma, or large cell carcinoma. In one embodiment of various aspects, the cancer is a solid tumor cancer.

In one embodiment of any aspect, the agent is a small molecule, an inhibitory nucleic acid, an antibody or antigen-binding fragment thereof, or antibody reagent, an inhibitory polypeptide, an antisense oligonucleotide, an immunotherapy, nanoparticle, or polymer.

In one embodiment of any aspect, inhibiting the level kills the SiglecF^{high} cell, and/or inhibits the rate at which the SiglecF^{high} cell is induced.

In one embodiment of any aspect, inhibiting the activity puts the cell into anergy, disrupts the functional interaction of the SiglecF^{high} cell and a tumor cell and/or tumor microenvironment, and/or disrupts the tumor-promoting function of a SiglecF^{high} cell.

In one embodiment of any aspect, inhibiting the migration disrupts the physical interaction of the SiglecF^{high} cell and a tumor cell and/or tumor microenvironment, disrupts the movement of the SiglecF^{high} cell towards a tumor cell and/or tumor microenvironment, and/or inhibits the tropism of the SiglecF^{high} cell.

In one embodiment of various aspects, the identifying of a population of SiglecF^{high} cells in a patient comprises assessing the gene profile of a sample from said patient and comparing it to the gene profile of SiglecF^{high} cells. The sample can be obtained from a biopsy of a lung, a biopsy of a lymph node, or bronchoalveolar lavage.

In one embodiment of any aspect, the method further comprises administering a second therapeutic. A second therapeutic can be a chemotherapeutic, radiation, an anti-tumor agent, or surgery.

### Definitions

The terms “decrease”, “reduce”, “inhibit”, or other grammatical forms thereof are used herein to mean a decrease by a statistically significant amount. In some embodiments, “reduce” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “inhibition” does not encompass a complete inhibition as compared to a reference level. “Complete inhibition” is a 100% inhibition as compared to a reference level. Where applicable, a decrease can be preferably down to a level accepted as within the range of normal for an subject without a given disease (e.g., cancer).

The terms “increased”, “increase”, “enhance”, or grammatical forms thereof are used herein to mean an increase by a statically significant amount. In some embodiments, the terms “increased”, “increase”, or “enhance”, can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, “subject” and “patient” are used interchangeably, and mean a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. non-small cell lung cancer or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors. A subject can also be one who has been identified as having a SiglecF^{high} cell population.

As used herein, the terms “treat,” “treatment,” or “treating,” refer to therapeutic treatments, wherein the object is to reverse, alleviate, ameliorate, inhibit, slow down or stop the progression or severity of a condition associated with a disease or disorder, e.g. lung cancer or other solid tumor cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic or pharmaceutical composition into a subject by a method or route which results in at least partial delivery of the agent at a desired site.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

As used herein, the term “DNA” is defined as deoxyribonucleic acid. The term “polynucleotide” is used herein interchangeably with “nucleic acid” to indicate a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

### BRIEF DESCRIPTION OF THE DRAWINGS

FIGS. 1A-1G present data that show lung tumors increase bone density in mouse models and cancer patients. (FIG. 1A) Fluorescence molecular tomography-based detection of OsteoSense signal (marking areas of active bone formation) in the femoral-tibial joint of KP lung tumor-bearing mice compared to their respective age- and sex-matched littermate tumor-free controls. Scale bar 5 mm. (FIG. 1B) Quantification of (FIG. 1A) (n=10-12 femoral-tibial joints per group). (FIG. 1C) Detection of OsteoSense signal as in (FIG. 1A) but in LLC lung tumor-bearing mice and their tumor-free controls (n=4 femoral-tibial joints per group). (FIG. 1D) Ex vivo confocal microscopy of representative OsteoSense signal and vasculature signal (labeled with anti-Sca-1, anti-CD31 and anti-CD144 mAbs) in the sternum of tumor-free mice (top) and KP lung tumor-bearing mice (bottom). Scale bar 500 μm. (FIG. 1E) 3D reconstruction of micro-computed tomography (CT) scans (left) and quantification of trabecular bone volume fraction (BV/TV) (right) in the distal femoral metaphysis of KP1.9 lung tumor-bearing and control mice (n=4 mice per group). Scale bar 500 μm. (FIG. 1F) CT-based trabecular bone density in patients with KRAS+ (positive) NSCLC and in control individuals. Left: representative axial non-contrast CT image of the 10th thoracic vertebra (T10) in a 53-year-old healthy woman who underwent non-contrast chest CT for cough and was found to have no abnormalities (control patient). Middle: a 53-year-old woman with KRAS+ NSCLC. Images are presented using the same window and level. The mean trabecular bone density of the region of interest depicted by a oval was calculated in Hounsfield Units (HU) for all investigated individuals. Right: quantitative data from control (n=35) and KRAS+ NSCLC (n=35) patients. (FIG. 1G) As in (FIG. 1F), but showing mean trabecular bone density of KRAS-(negative) NSCLC patients (n=35) and matched controls (n=35). All figures show mean±SEM. Statistical significance was calculated using an unpaired t-test. *p<0.05, **p<0.01, ***p<0.001. Abbreviations: AdCre: adenovirus-Cre; KP: Kras and p53 mutant lung tumors; LLC: Lewis Lung Carcinoma; NSCLC: non-small cell lung cancer.

FIGS. 2A-2E present data that show lung tumors increase osteoblast activity in mice. (FIG. 2A) Representative Goldner's Trichrome staining of distal femur sections from a tumor-free mouse (top) and a KP lung tumor-bearing mouse (bottom) (n=4 mice per group). Osteoblasts are indicated with arrowheads. Scale bar 1 mm. See FIG. S6A-D. (FIG. 2B) Number of osteoblasts per bone surface in distal femur trabecular bone from the same mice as in (FIG. 2A) (n=4 mice per group). (FIG. 2C) Flow cytometry-based quantification of the percentage of bone marrow Ocn-YFP+ cells isolated from tumor-free mice and KP lung tumor-bearing OcnCre;Yfp mice (n=6 mice per group). Ocn-YFP+ cells were defined as 7AAD− Lin− CD45− CD31− Ter119− YFP+. (FIG. 2D) Representative von Kossa staining (left) and quantification of mineralized bone (% von Kossa area, right) in femurs from the same mice as in (FIG. 2A) (n=4 mice per group). Scale bar 1 mm. (FIG. 2E) Left: representative images of bone formation in trabecular bone of femurs from tumor-free mice and KP lung tumor-bearing mice. Double arrows depict distance between sequential injections of calcein and demeclocycline. # denotes trabecular bone. Scale bar 10 μm. Right: quantification of mineral apposition rate (n=3-4 mice per group). See FIGS. 13A and 13B for additional measurements. All figures show mean±SEM. Statistical significance was calculated using an unpaired t-test. *p<0.05, **p<0.01. Abbreviations: KP: Kras and p53 mutant lung tumors; Ocn: osteocalcin; YFP: yellow fluorescent protein.

FIGS. 3A-3F present data that show Ocn+ cells foster a tumor-promoting neutrophil response in mice. (FIG. 3A) Comparison of lung weight (proxy of tumor burden) in KP1.9 tumor-bearing mice with reduced numbers of Ocn+ cells (OcnCre;Dtr mice treated with DT) or in tumor-bearing control mice (mice lacking Cre or Dtr and treated with DT). DT was administered three weeks after tumor injection, i.e. when tumors were established. OcnCre;Dtr mice that did not receive DT were used as additional controls. Data show delta lung weights (pre/post DT treatment) and are pooled from four separate experiments (n=8-31 mice per group). Statistical significance was calculated using one-way ANOVA and Tukey's multiple comparisons test. (FIG. 3B) Tumor burden in control mice or in mice with reduced numbers of Ocn+ cells. Mice are defined as in (FIG. 3A). Left: representative H&E-stained lung tissue sections. Scale bar 1 mm.; right: quantification of percent change in tumor area following DT treatment. Statistical significance was calculated using an unpaired t-test. Data are pooled from three independent experiments (n=13 mice per group). (FIG. 3C) Ex vivo flow cytometry-based evaluation of neutrophils, monocytes and macrophages in lungs of tumor-bearing control mice or in mice with reduced numbers of Ocn+ cells, as defined in (FIG. 3A). Data were normalized to control (Ocn-sufficient) tumor-bearing mice and pooled from three independent experiments (n=14-25 mice per group). Statistical significance was calculated using multiple t-tests. (FIG. 3D) Fold change in volume of KP lung tumor nodules pre and post anti-Gr-1 or isotype mAb treatment. Tumors were detected noninvasively by micro-computed tomography (n=2-3 tumor nodules per mouse, 4-5 mice per group). Statistical significance was calculated using an unpaired t-test. (FIG. 3E) Number of CD11b+Ly6G+ neutrophils per ml blood in KP1.9 tumor-bearing control mice or in mice with reduced numbers of Ocn+ cells. Mice are defined as in (FIG. 3A). Mice were analyzed three days after DT treatment (n=4-5 mice per group) and cells were quantified by flow cytometry. Tumor-free OcnCre;Dtr mice were used as additional control. Statistical significance was calculated using one-way ANOVA and Tukey's multiple comparisons test. (FIG. 3F) Tumor-bearing mice with reduced numbers of Ocn+ cells were parabiosed with mice that had either normal numbers of Ocn+ cells (control parabiont) or reduced numbers of Ocn+ cells (OcnCre;Dtr parabiont). Left: outline of the parabiosis experiments. Middle: quantification by flow cytometry of lung tumor-infiltrating granulocytes in tumor-bearing OcnCre;Dtr mice. Right: lung weight of the same mice (n=4-6 mice per group). Statistical significance was calculated using an unpaired t-test. All figures show mean±SEM. *p<0.05, **p<0.01, ***p<0.001, n.s. not significant. Abbreviations: DT: diphtheria toxin; KP: Kras and p53 mutant lung tumors; Ocn: osteocalcin.

FIGS. 4A-4D present data that show Ocn+ cell-driven neutrophils exhibit discrete phenotypes. (FIG. 4A) Flow cytometry-based detection (left) of Ly-6G+ SiglecFhigh or low neutrophils from healthy lung tissue (top) and KP1.9 lung tumors (bottom). Plots are shown for gated live CD45+ CD11b+ cells. Representative cytospin images (right) are from FACS-sorted populations further stained with H&E. Scale bar 10 μm. (FIG. 4B) Fold change Ly-6G+ SiglecFhigh and Ly-6G+ SiglecFlow cell number in lungs from tumor bearing-mice when compared to tumor-free mice. Cells were assessed by flow cytometry (n=6 mice per group). (FIG. 4C) Representative SiglecF mAb staining on cryo-preserved KP lung tumor tissue. Tumor areas are highlighted by dotted lines. Scale bar 50 μm. (FIG. 4D) Flow cytometry-based quantification of Ly-6G+ SiglecFhigh and Ly-6G+ SiglecFlow cells in tumor-bearing lungs of mice with either preserved Ocn+ cells (control mice treated with DT) or reduced numbers of these cells (OcnCre;Dtr mice treated with DT) (n=7-9 mice per group). (FIG. 4E) Ability of CD45.1+ Lin− cKit+ hematopoietic precursors to produce tumor-infiltrating SiglecFhigh and SiglecFlow neutrophils upon transfer into KP tumor-bearing CD45.2+ recipient control mice or mice with reduced numbers of Ocn+ cells. Mice were treated as in (FIG. 4D). Results are shown as fold change relative to control mice. All figures show mean±SEM and significance values were calculated using multiple t-tests. *p<0.05, **p<0.01, ****p<0.0001, n.s. not significant. Abbreviations: KP: Kras and p53 mutant lung tumors; Lin.: Lineage; Ocn: osteocalcin.

FIGS. 5A-5F present data that show SiglecFhigh neutrophils exhibit tumor-promoting phenotypes and functions in mice. (FIG. 5A) Volcano plot showing differential gene expression between T-SiglecFhigh and T-SiglecFlow cells. Genes with false discovery rate (FDR)<5% and an absolute fold change (FC) >2 are highlighted, denoting down- and up-regulated genes, respectively, in T-SiglecFhigh cells versus T-SiglecFlow cells. Statistical analysis is outlined in materials and methods. (FIG. 5B) Average expression levels of genes involved in angiogenesis, myeloid cell recruitment, tumor proliferation, cytotoxicity, extracellular matrix remodeling and immunosuppression in T-SiglecFhigh, T-SiglecFlow and H-SiglecFlow cells. (FIG. 5C) Representative histogram (left) and quantification of gMFI (right) of ROS activity, measured by rhodamine 123 fluorescence (oxidized Dihydroamine 123) using flow cytometry, in T-SiglecFhigh, T-SiglecFlow and H-SiglecFlow cells (n=4-5 mice per group). (FIG. 5D) Representative flow cytometric dot plots showing CD11b+F4/80+ macrophages derived from splenic monocytes and cultured with T-SiglecFhigh, T-SiglecFlow or H-SiglecFlow cells (all gated on live CD45+ cells). Cultures in medium alone or with CSF-1 were used as negative and positive controls, respectively. Mean macrophage frequency±SEM are shown in parentheses. (FIG. 5E) Quantification of macrophage numbers as in (D) with 4-5 replicates per condition. (FIG. 5F) KP1.9 tumor growth in mice following tumor cell co-injection with either T-SiglecFhigh, T-SiglecFlow or H-SiglecFlow cells (n=4-5 mice per group). (FIG. 5G) Survival (Kaplan-Meier) plots of lung adenocarcinoma patients. Patients were stratified based on high (SiglecFhigh, top 25%) versus low (SiglecFlow, bottom 25%) expression of the humanized SiglecF neutrophil gene signature. p valued calculated using Cox regression method. See Methods and materials for details. Panels (FIG. 5C-5F) show mean±SEM. **p<0.01, ****p<0.0001, n.s. not significant. Statistical values were calculated using one-way ANOVA (5C and 5E) or two-way ANOVA (FIG. 5F). Abbreviations: CSF-1: colony-stimulating factor-1; gMFI: geometric mean fluorescence intensity; H: Healthy; KP: Kras and p53 mutant lung tumors; ROS: reactive oxygen species; T: Tumor.

FIGS. 6A-6D present data that show sRAGE contributes to the osteoblast-induced neutrophil response. (FIG. 6A) Bone marrow cells were cultured in osteogenic medium with serum from either tumor-free or lung tumor-bearing mice. Osteoblastic colonies were detected by alkaline phosphatase (ALP) staining. Graph shows the change in ALP+ (osteoblastic) colonies upon culture with serum from tumor-bearing mice compared to serum from tumor-free mice (n=4 replicates per condition). (FIG. 6B) Protein content was investigated in the blood of lung tumor-bearing (TB) and tumor-free (TF) mice using protein arrays. Heat-map shows relative protein content that was detectable above background levels and reproducibly altered between two individual protein arrays. Heat map shows pooled results from the two arrays and are normalized to blood from tumor-free mice. Scale: 0.5- to 2.0-fold change. (FIG. 6C) Osteoblastic colony formation measured as in (FIG. 6A) but using bone marrow cells exposed or not to sRAGE. Graph shows the change in ALP+ (osteoblastic) colonies upon exposure to sRAGE compared to serum alone (n=6 replicates per condition). (FIG. 6D) Flow cytometric evaluation of CXCR2 expression on developing neutrophils derived from bone marrow HSPCs of tumor-free mice. The cells were cultured without (left) or with (right) ST2 stromal cells, and with increased amounts of sRAGE (n=3 replicates per condition). Abbreviations: CXCR2: C-X-C chemokine receptor 2; HSPCs: hematopoietic stem and progenitor cells; sRAGE: soluble receptor for advanced glycation endproducts.

FIGS. 7A-7C present data that show lung adenocarcinoma development in KP mice. (FIG. 7A) In KP mice lung tumors are initiated through i.t. delivery of AdCre virus. (FIG. 7B) Representative H&E staining of lung lobe sections from KP tumor-bearing mice (+AdCre) post-tumor induction. Scale bar 2.5 mm. (FIG. 7C) Lung weight of KP lung tumor-bearing and tumor-free mice from OsteoSense experiment presented in FIG. 1A-B (n=5-6 mice per group). Statistical significance was calculated by an unpaired t-test. ***p<0.001; AdCre: Adenovirus-Cre.

FIGS. 8A-8E present data that show OsteoSense FMT analysis. (FIG. 8A) Experimental outline of OsteoSense injections into KP or LLC lung tumor-bearing or tumor-free mice and subsequent FMT readout. (FIG. 8B) Representative FMT images of OsteoSense signal in the femoral-tibial joint of LLC tumor-bearing mice and tumor-free control mice. (FIG. 8C) Representative ex vivo FMT OsteoSense signal in the rib cage of KP tumor-bearing (+AdCre) and tumor-free mice (−AdCre). (FIG. 8D) Representative ex vivo FMT images of long bones in mice with or without tumors (±AdCre) and injected or not with OsteoSense (±OsteoSense). (FIG. 8E) Ex vivo OsteoSense signal quantification in bones (n=24-25 bones per group) in various compartments including femoral-tibial joint, elbow joint, ribs sternum, vertebrae and pelvic bone. OsteoSense-injected mice as in (FIG. 8A). Statistical significance was calculated by an unpaired t-test. ****p<0.0001; AdCre: Adenovirus-Cre; FMT: Fluorescence-mediated tomography.

FIGS. 9A-9E present data that show KP lung tumor cells do not detectably metastasize to the bone. (FIG. 9A) Representative images of lung tumor tissue (left), tumor-free lung tissue (middle) and femur from a KP lung tumor-bearing mouse (right). Scale bar 100 μm. (FIG. 9B) Scoring of the presence of tumor cells in sections described in (FIG. 9A). 10 sections were scored per mouse (20× magnification), n=8 mice per group. (FIG. 9C) Detection of recombined p531 lox (only present in tumor cells) by PCR. Recombined p531 lox band: 612 bp; WT band: 288 bp; background band: 400 bp. KP.19 tumor cells were titrated to determine the PCR's detection sensitivity (1 cell approximately contains 6 pg of DNA). (FIG. 9D-9E) P53 PCR from DNA isolated from whole bone marrow (FIG. 9D) or calvarial bone (FIG. 9E) of KP lung tumor-bearing mice and compared to DNA isolated from KP1.9 tumor cells as in (FIG. 9C).

FIGS. 10A and 10B present data that show OsteoSense microscopy analysis. (FIG. 10A) Additional representative confocal microscopy images as presented in FIG. 1D of OsteoSense and vasculature signal (labeled with anti-Sca-1, anti-CD31 and anti-CD144 mAbs) in the sternum of KP1.9 lung tumor-bearing or tumor-free mice. Scale bar 500 μm. (FIG. 10B) Representative ex vivo confocal microscopy images of the femur from tumor-free (left) or KP tumor-bearing mice (right) showing OsteoSense signal. Scale bar 500 μm.

FIGS. 11A-11I present data that show CT analysis. Lung weight (proxy for tumor burden) and bone parameters (CT analysis of femurs) were measured in mice bearing KP 1.9 lung tumors and tumor-free mice. (FIG. 11A) Lung weight of mice (n=4 mice per group) analyzed for CT, Goldner's Trichrome (FIG. 2A), von Kossa staining (FIG. 2D). (FIG. 11B) Diagram of the trabecular and cortical bone areas that were scanned using CT in FIG. 1E and FIGS. 11C-11I. (FIG. 11C) Representative images of CT scans of trabecular bone from tumor-free (left) and tumor-bearing mice (right). Scale bar 1 mm. (FIG. 11D) Bone mineral density (BMD) (n=4 mice per group). (FIG. 11E) Trabecular number (Tb.N) (n=4 mice per group). (FIG. 11F) Trabecular thickness (Tb.Th) (n=4 mice per group). (FIG. 11G) Trabecular separation (Tb.Sp) (n=4 mice per group). (FIG. 11H) Representative images of CT scans of cortical bone from tumor-free (top) and KP1.9 lung tumor-bearing mice (bottom). Scale bar 1 mm. (FIG. 11I) Combination of all parameters investigated in the femurs using CT and presented in panels FIG. 11A to 11H. Statistical significance was calculated by an unpaired t-test. *p<0.05, **p<0.01, ***p<0.001.

FIGS. 12A-12D present data that show bone histomorphometry analysis. Cellular composition of trabecular bone was evaluated in KP1.9 lung tumor-bearing and tumor-free mice (see also FIG. 2A). (FIG. 12A) Osteoid surface/bone surface (n=4 mice per group). (FIG. 12B) Osteoclast number/bone surface (n=4 mice per group). (FIG. 12C) Eroded surface/bone surface (n=4 mice per group). (FIG. 12D) Representative magnification of the epiphyseal plate in Goldner's Trichrome stained femur sections from tumor-free (top) and tumor-bearing mice (bottom). Scale bar 1 mm. Statistical significance was calculated by an unpaired t-test. **p<0.01, n.s. not significant.

FIGS. 13A and 13B present data that show in vivo bone histomorphometry. See FIG. 2E. (FIG. 13A) Mineralized surface over bone surface in femur sections of tumor-free and tumor-bearing mice (n=3-4 mice per group) based on calcein and demeclocycline labeling. (FIG. 13B) Bone formation rate per bone surface area per day for mice as in (FIG. 13A). Statistical significance was calculated by an unpaired t-test. *p<0.05.

FIGS. 14A-14C present data that show RNAseq analysis of sorted osteoblasts reveals distinct tumor-induced changes in gene expression. (FIG. 14A) Generation of KPOcn-GFP mice and representative flow cytometry-based quantification to identify GFP positive cells that reflect osteoblasts in bone tissue of these mice. (Pre-gated on 7AAD⁻ CD45⁻ cells). (FIG. 14B) RNAseq analysis of Ocn-GFP positive cells obtained through flow cytometry based cell sorting for Lin⁻ CD45⁻Ter119⁻GFP⁺ cells from KPOcn-GFP tumor-bearing versus littermate tumor-free control mice. Presented are upregulated (in red) and downregulated genes (in blue) in Ocn-GFP osteoblastic cells. Overexpression of the Fos12 transcription factor leads to a high trabecular mass phenotype and can drive Ocn and Col1a2 expression (26, 27). Conversely, Dlk1 and Ndrg1 may negatively regulate bone density: osteoblast-specific Dlk1 overexpression results in decreased bone mineral density, trabecular bone volume and osteoblast frequency (28), and knocking out Ndrg1 leads to higher trabecular bone mass and reduced osteoclast activity (29). Thus, Ocn⁺ cells in tumor-bearing mice simultaneously upregulate bone stimulatory factors (Fos12) and reduce bone inhibitory agents (Dlk1 and Ndrg1). Decreased Cxc14 expression in OCN⁺ cells from tumor-bearing mice may also be relevant since CXCL14 has been shown to interact with CXCR4 and synergize with CXCL12 binding to CXCR4 (85). CXCL12-CXCR4 interactions are critical in neutrophil retention in the bone marrow; thus, CXCL14 downregulation by OCN⁺ cells could be involved in neutrophil exit from the bone marrow. (FIG. 14C) Table of downregulated (left) and upregulated genes (right) in Ocn⁺ cells shown in (FIG. 14B). FC: fold change in gene expression versus tumor-free mice (in log 2 scale).

FIGS. 15A-15F present data that show reduction of Ocn+ cells in OcnCre;Dtr/Yfp mice following diphtheria toxin (DT) treatment. (FIG. 15A) Experimental outline for DT-mediated Ocn+ cell depletion in mice with or without cancer. (FIG. 15B) Delta body weight of mice treated as in (A) (n=4-9 mice per group). Statistical significance was calculated using One-way ANOVA followed by Tukey's multiple comparisons test. (FIG. 15C) Bone marrow cells from DT-treated OcnCre;Dtr and OcnCre;Yfp/Yfp mice were stained by flow cytometry to identify YFP+ Ocn+ cells. Representative dot plots are shown (pre-gated on 7AAD− CD45− CD31− cells). (FIG. 15D) Representative Ocn IHC staining on bone sections of control and OcnCre;Dtr/Yfp mice. Scale bar 50 μm. (FIG. 15E) Representative ex vivo confocal microscopy images identify Ocn+ cells in bone tissue (femur) of OcnCre;Dtr/Yfp mice treated or not with DT and in DT-treated control mice. The bone is visualized by OsteoSense and the vasculature by antibody staining (stained in vivo with anti-CD31, anti-Sca1 and anti-CD144 mAbs). Scale bar 500 μm. (FIG. 15F) Representative H&E staining of decalcified paraffin-embedded femur sections of DT-treated control (left) and OcnCre;Dtr/Yfp mice (right) with magnification of Ocn+ cells. Scale bar 50 μm. n.s. not significant.

FIGS. 16A-16C present data that show Diphtheria toxin (DT) control experiments in bone marrow of DT-treated Cd169^{Dtr} mice. (FIG. 16A) Lung weight as proxy of tumor burden in Cd169^{Dtr} mice or control mice (n=9 mice per group). (FIG. 16B) Flow cytometry-based evaluation of bone marrow macrophage cell numbers per femur after DT-treatment in a subset of mice in (FIG. 16A) (n=4 mice per group). (FIG. 16C) Flow cytometry-based evaluation of bone marrow neutrophil cell numbers per femur after DT-treatment in a subset of mice in (FIG. 16A) (n=4 mice per group). Statistical significance was calculated by an unpaired t-test. ***p<0.001; n.s. not significant.

FIG. 17 present data that show KP tumor-infiltrating lymphocyte counts in DT-treated control and Ocn^{Cre;Dtr} mice. Lymphocyte populations including T cells (CD3⁺, CD3⁺CD4⁺, CD3⁺CD8⁺), B cells (B220⁺CD19⁺) and NK cells (CD49b⁺NK1.1⁺) were quantified ex vivo by flow cytometry. Results are shown relative to control mice (n=13-19 mice per group). Multiple t-tests were used to calculate statistical significance. **p<0.01; n.s. not significant.

FIGS. 18A-18D present data that show NK cell depletion does not restore tumor burden in osteoblast-reduced lung tumor-bearing mice. (FIG. 18A) Experimental layout of Ab-based NK1.1 cell depletion in KP1.9 tumor-bearing mice with either normal or reduced osteoblasts. (FIG. 18B) Flow cytometry-based verification of NK cell depletion in lung tumor tissue of Ocn^{Cre;Dtr} mice treated as in (FIG. 18A). Representative dot plots are shown. (FIG. 18C) Quantification of CD49b⁺ Nkp46⁺ cells per mg lung tissue of mice treated as in (FIG. 18A) (n=7-9 mice per group). (FIG. 18D) Lung weight as a proxy for tumor burden in mice treated as in (FIG. 18A) (n=7-9 mice per group). Statistical significance was calculated using One-way ANOVA followed by Tukey's multiple comparisons test. *p<0.05, **p<0.01, ****p<0.0001, n.s. not significant; DT: diphtheria toxin.

FIGS. 19A-19F present data that show controls for DT- and Cre-DTR-mediated effects on hematopoietic cells. (FIG. 19A-19B) Percentage (FIG. 19A) and cell number per femur (FIG. 19B) of neutrophils, quantified by flow cytometry in bone marrow of wild-type (WT) mice treated or not with DT, treated as in FIG. S9A (n=3-4 mice per group). Statistical significance was calculated by an unpaired t-test. (FIG. 19C) Splenocytes from WT or Ocn^{Cre;Dtr} mice were incubated in vitro with increasing doses of DT. Percentage of CD11b⁺ cells among CD45⁺ cells was calculated by flow cytometry after 20 h of treatment. Results are normalized to untreated cells from each genotype (n=3 replicates per group). (FIG. 19D) Percentage of dead 7AAD⁺ CD11b⁺ cells was calculated by flow cytometry as in (FIG. 19C) (n=3 replicates per group). (FIG. 19E-19F) Positive control experiments to verify DT's ability to kill diphtheria toxin receptor (DTR)⁺ cells in vitro. Splenocytes from WT or Cd11c^{Dtr} mice were treated with DT (n=2 replicates per group) and the percentage of CD11c⁺ (DTR⁺) cells (FIG. 19E) and CD3⁺ (DTR⁻) cells (FIG. 19F) were calculated by flow cytometry as in (FIG. 19C). n.s. not significant; DT: diphtheria toxin.

FIGS. 20A and 20B present data that show controls for neutrophil depletion. Also see FIG. 3D. (FIG. 20A) Representative contour plots of flow cytometry-based evaluation of neutrophils in isolated lung tumor nodules from KP mice. Neutrophils were defined as live CD45⁺ CD11b⁺ Ly-6G⁺ CD11c⁻ Ly-6C⁺ cells. (FIG. 20B) The percent of neutrophils defined as in (A) was investigated in lung tumor nodules (left) and blood (right) of KP mice treated with anti-Gr-1 or isotype control mAbs, respectively (n=6 tumor nodules/group; n=3-5 mice/group). Statistical significance was calculated by an unpaired t-test. **p<0.01, ****p<0.0001.

FIGS. 21A and 21B present data that show SiglecF^{high} neutrophils (CD11b⁺ Ly-6G⁺) in lung tumors expand during tumor progression. (FIG. 21A) Ratio between lung SiglecF^{high} and SiglecF^{low} neutrophils (measured by flow cytometry) plotted against lung weight (proxy of tumor burden) of KP 1.9 lung tumor-bearing mice (n=23 mice) and linear regression was performed. (FIG. 21B) Representative dot plots showing SiglecF vs Ly-6G expression in tumor-free lungs (left), lungs with low KP tumor burden (middle) and lungs with high KP tumor burden (right).

FIG. 22 presents data that show phenotyping of SiglecF^{high} neutrophils in the tumor microenvironment by flow cytometry reveals a neutrophil-like phenotype. Representative flow cytometry histograms showing forward and side scatter profiles, as well as expression of five cell surface myeloid-associated markers, for: Ly-6G⁺ SiglecF^{high} cells in KP tumor-bearing mice, Ly-6G⁺ SiglecF^{low} cells in KP tumor-bearing mice (neutrophil-like), Ly-6G⁺ SiglecF^{low} cells in tumor-free mice (neutrophil-like), Ly-6G⁻ Ly-6C⁺ cells in KP tumor-bearing mice (monocyte-like), CD11b^{low} Ly-6G⁻ SiglecF^{high} CD11c⁺ cells in KP tumor-bearing mice (alveolar macrophages), Ly-6G⁻ SiglecF^{high} cells in KP tumor-bearing mice (eosinophil-like), and remaining Ly-6G⁻ Ly-6C^{low} SiglecF^{low} CD11b⁺ cells (including macrophages/dendritic cells). Grey histograms indicate unstained or fluorescence-minus-one (FMO) control samples. According to this analysis, Ly-6G⁺ SiglecF^{high} cells closely resemble the neutrophil populations found in both healthy and tumor tissue and are distinct from other SiglecF⁺ cells in the tumor microenvironment, including alveolar macrophages and eosinophils.

FIGS. 23A-23F present data that show SiglecF⁺ cells in tumor-free areas resemble alveolar macrophages. See also FIG. 4C. (FIG. 23A) IHC based anti-SiglecF mAb validation on murine spleen sections (positive cells are highlighted with arrowheads). Scale bar 100 μm. (FIG. 23B) Ly-6G⁺ cells in representative KP lung tumor tissue. Tumor area is highlighted with a dotted line. Scale bar 50 m or 500 m. (FIG. 23C) Representative SiglecF IHC staining in lung tumor-adjacent tissue of KP1.9 tumor-bearing mice shows cells with macrophage like phenotype (arrowheads). Tumor area is highlighted with a dotted line. Scale bar 20 m. (FIG. 23D) Ly-6G⁺ cells and Ly-6G⁻ cells, the latter with macrophage-like morphology, in representative tumor-adjacent lung tissue. Scale bar 10 μm. (FIG. 23E) Representative H&E stained cytospins of Ly-6G⁺ SiglecF^{high} cells (top) and alveolar macrophages (bottom). Scale bar 10 m. (FIG. 23F) SiglecF IHC staining in tumor-free lung tissue. Representative positive cells are highlighted with arrowheads. Scale bar 50 m.

FIGS. 24A and 24B present data that show CD45.1 myeloid and lymphocyte progeny in osteoblast sufficient and reduced mice. Fate mapping experiment to evaluate CD45.1⁺ Lin⁻ cKit⁺ hematopoietic precursor's ability to produce myeloid and lymphocyte progeny in lung tumors of CD45.2⁺ Ocn^{Cre;Dtr} or CD45.2⁺ control mice (see also FIG. 4E). (FIG. 24A) Percent of CD11b⁺ Ly-6G⁻ cells in lung tumor tissue of Ocn^{Cre;Dtr} vs control mice (n=7-9 mice per group). Data are relative to those observed in host control mice. (FIG. 24B) Percent of B220⁺ cells in lung tumor tissue of Ocn^{Cre;Dtr} vs control mice (n=7-8 mice per group), as shown in (FIG. 24A). Statistical significance was calculated by an unpaired t-test. n.s. not significant.

FIGS. 25A-25D present data that show phenotyping of SiglecF^{high} cells in the tumor microenvironment by single cell RNAseq analysis. See also FIGS. 5A and 5B. (FIG. 25A) SiglecF⁺ gene signature score based on single cell analysis of neutrophils from KP1.9 lung tumors or healthy controls. Hematopoietic cells were FACS sorted and 6,020 cells were defined as neutrophils. (FIG. 25B) Volcano plot showing significantly differential gene expression between T-SiglecF^{high} and H-SiglecF^{low} cells. (FIG. 25C) Volcano plot highlighting significantly differential gene expression between T-SiglecF^{low} and H-SiglecF^{low} cells. (FIG. 25D) Heat-maps showing relative gene expression of levels of transcription factors (left) and cytokine or cytokine receptors (right) in T-SiglecF^{high}, T-SiglecF^{low} and H-SiglecF^{low} cells. FC: fold change; FDR: false discovery rate; TPM: transcripts per million.

FIGS. 26A and 26B present data that show gene set enrichment analysis of T-SiglecF^{high} cells. (FIG. 26A) Positively enriched gene sets in T-SiglecF^{high} vs T-SiglecF^{low} cells (left) and enrichment plots of selected gene sets (right). (FIG. 26B) Same as in (FIG. 26A) but for negatively enriched gene sets.

FIGS. 27A and 27B present data that show differentiation of neutrophils in the blood before tumor entry. (FIG. 27A) Neutrophils were sorted from blood of KP 1.9 tumor-bearing or tumor-free mice (n=3-8 mice per group). Genes that are upregulated in SiglecF^{high} cells at the tumor site (including Siglecf, Xbp1 and Clec4n; data not shown) were significantly increased in neutrophils in the blood of tumor-bearing mice compared to tumor-free controls. (FIG. 27B) Representative flow cytometry-based histograms of neutrophils in the blood of mice with KP1.9 lung tumors or controls. FMOs are shown. Neutrophils were defined as live CD45⁺CD11b⁺Ly6G⁺ cells. Statistical significance was calculated using an unpaired t-test. *p<0.05, ***p<0.001, n.s. not significant.

FIGS. 28A and 28B present data that show High expression of a SiglecF^{high} neutrophil signature is associated with decreased survival in lung adenocarcinoma patients across databases. Survival (Kaplan-Meier) plots of lung adenocarcinoma patients from five individual databases (34, 35). Patients were stratified based on high (top 25%) versus low (bottom 25%) expression of the humanized (FIG. 28A) SiglecF^{high} or (FIG. 28B) SiglecF^{low} neutrophil gene signature. For each database, 25% of patients corresponds to the following numbers: DFCI (n=21), Ladanyi (n=32), MI (n=44), MSKCC (n=26), and HLM (n=19). p values were calculated using a log-rank test. See also FIG. 5G. T: Tumor.

FIGS. 29A-29C present data that show sRAGE is increased in the serum of tumor-bearing mice. See also FIG. 6B. (FIG. 29A) Representative protein array membranes, incubated with serum of tumor-free (top) or tumor-bearing (bottom) mice, revealed elevated signal intensities for sRAGE in the blood of mice with lung tumors. (FIG. 29B) Quantitative data of protein array spots as presented in (FIG. 29A), normalized by signal intensity of array reference spots. (FIG. 29C) Measurement of sRAGE in murine serum by ELISA (left, n=10-11 mice per group) and corresponding lung weight as proxy for tumor burden (right, n=10-11 mice per group). Statistical significance was calculated using an unpaired t-test. **p<0.01, ***p<0.001, ****p<0.0001.

FIGS. 30A-30C present data that show neutrophils expand in blood of tumor-bearing mice, but do not upregulate SiglecF. (FIG. 30A) Number of neutrophils (defined as Ly-6G+) per ml of blood in tumor-free mice or in mice with lung adenocarcinoma (so-called KP tumors carrying Kras mutations and lacking P53). (FIG. 30B) Same as in A but specifically for SiglecF^{low} neutrophils. (FIG. 30C) Same as in FIG. 30A but specifically for SiglecF^{high} neutrophils. n.s., not significant; **p<0.01.

FIGS. 31A and 31B present data that show SiglecF^{high} neutrophils are detectable in bronco-alveolar lavage (BAL) fluids collected from mice with lung cancer. (FIG. 31A) Number of SiglecF-high neutrophils in BAL fluids from tumor-free mice or from mice with KP lung adenocarcinoma. (FIG. 31B) Representative strategy used for ex vivo flow cytometry-based detection of SiglecF-high neutrophils in BAL fluids (gated on 7AAD⁻ Lin⁻ CD45+ cells). ****p<0.0001.

### DETAILED DESCRIPTION

The invention described herein related to, in part, the discovery that lung tumors, specifically non-small cell lung cancer tumors, induce the production of a osteocalcin-expressing (Ocn⁺) osteoblastic cells, resulting in an increase of bone density in cancer patients. Surprising, it was determined that these induced Ocn⁺ cells gave rise to a distinct subset of tumor-infiltrating SiglecF^{high} neutrophils. Findings presented herein show that SiglecF^{high} neutrophils exhibit tumor-promoting functions and promote tumor progression. Elimination of the SiglecF^{high} neutrophil population can reduce the capacity for a tumor microenvironment to protect that tumor, thus targeting these neutrophils can be an effective anti-cancer strategy. Presented herein is the SiglecF^{high} neutrophils gene profile, which is useful in targeting said SiglecF^{high} neutrophil population for example, for cell death.

SiglecF^{high} Neutrophils

A neutrophil is an abundant type of white blood cell derived from stem cells within the bone marrow and play an essential function in the inate immune system. These cells are recruited to sites of inflammation caused by an infection, environmental exposures, and certain cancer by chemical signals, for example Interleukin-8, C5a, fMLP, Leukotriene B4, and H₂O₂. Neutophil recruitment is the hallmark of acute inflammation; a neutrophil will secrete proteolytic enzymes at the site of inflammation to improves its mobility and allows for the cell to envelop a bacterial cell (e.g., to minimize or eliminate the infection).

In cancer, neutrophils have been shown to exhibit tumor-protecting functions in the microenvironment. Neutrophils have been shown to expand in the solid tumor microenvironment and systemically, promote tumor initation, growth, and metastasis, and can polarize the tumor microenvironment, such that anti-cancer are inefficient in treating the tumor. Patients with tumors that present with elevated neutrophils have a poor prognosis.

Described herein is a distinct subset of neutrophils associated with cancer (e.g., non-small cell lung cancer) that promotes tumor progression in the lung, and referred to as SiglecF^{high} neutrophils. These cells are induced by OCN⁺ cells, which are upregulated in the bone due to cancer, for example, lung cancer. SiglecF^{high} neutrophils are defined by their gene profile, which is distinct from other neutrophils. In contrast, a SiglecF^{low} cell does not exhibit a tumor-promoting function, thus this is specific to a SiglecF^{high} cell. Importantly, this subset of neutrophils are only found in tumors, and not present in healthy tissue, indicating that SiglecF^{high} neutrophils are tumor-specific.

In one embodiment, a SiglecF^{high} neutrophil is a tumor-infiltrating neutrophil. As used herein, “tumor-infiltrating” refers to a cell (e.g., a neutrophil) that has left the bloodstream and migrated to the site of a tumor, where it can then integrate into the tumor (i.e., be found in between tumor cells). Histologic analysis of a tumor sample (e.g., a biopsy of the tumor) can readily identify tumor infiltrating cells via distinct cell morphology of the tumor infiltrating cells, or by determining the gene expression of the tumor (e.g., by microarray analysis or RNA sequencing).

The 50 genes that are most correlated with the SiglecF^{high} neutrophils gene profile are listed in Table 1. A cell is considered a SiglecF^{high} cell if its gene profile comprises at least 20% of the genes identified as being in the SiglecF^{high} cell gene profile.

Human orthologs of the murine genes identified in the gene profile for a SiglecF^{high} cell can be used to identify a human SiglecF^{high} cell. Human orthologs of the SiglecF^{high} cell gene profile are listed in Table 2. Orthologs for other species can be used to identified a SiglecF^{high} cell of that given specie. Homologs, paralogs, and/or orthologs of genes (e.g., a gene within the SiglecF^{high} cell gene profile) are readily identified for a given species by one of skill in the art, e.g., using the NCBI ortholog search function or searching available sequence data for a given species for sequence similar to a reference sequence.

Identification of SiglecF^{high} neutrophils

In one embodiment, a population of SiglecF^{high} cells is identified in a patient prior to administration of an agent that inhibits a SiglecF^{high} cell. To identify a population of SiglecF^{high} cells in a patient, a sample from said patient is taken, analysed, and the gene profile of the cells within the sample is identified. The gene profile of the cells within a sample is compared to the gene profile of a SiglecF^{high} cell. In one embodiment, a cell is considered a SiglecF^{high} cell if at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% or more of its gene profile matches that of the SiglecF^{high} cell gene profile (e.g., at least 20% of the genes comprised in the gene profile of a sample are comprised within the gene profile of a SiglecF^{high} cell gene profile). It is not required that the SiglecFhigh cell be a neutrophil, but rather, any cell type that exhibits similarity, as described above, to the SiglecF^{high} gene profile.

A sample can be obtained from a patient using various techniques known in the art. In one embodiment, a sample is obtained from a patient by a lung biopsy, a lymph node biopsy, or a bronchoavlveolar lavage. A sample can be obtained prior to a patient be diagnosed with cancer. Alternatively, a sample can be obtained after a patient has been diagnosed with cancer.

Procedures used to obtain a lung sample are known in the art and can be performed by a skilled person. Exemplary procedures used to obtain a lung biopsy sample include percutaneously (through the skin with guidance provided by, for example, a CT scan), via bronchoscopy guided by, for example, an ultrasound, via open surgery, or video-assisted thoracoscopic surgery. Procedures used to obtain a lymph node samples are known in the art and can be performed by a skilled person and can include a fine-needle aspiration biopsy, a core needle biopsy, or a open surgical biopsy. Bronchoavlveolar lavage is known in the art and can be performed by a skilled person. Bronchoavlveolar lavage, also known as “lung washing” is a procedure that in which fluid is added to the lung tissue and then collected. The collected fluid can contain cells from the lung, and is examined.

Methods for assessing the gene profile of cells within a sample obtained from a patient include, but are not limited to, RNA sequencing and DNA microarray; these techniques are known in the art. Comparisons between the gene profile of the sample obtained from a patient and a SiglecF^{high} cell can be done, for example, by using Metabolic gEne RApid Visualizer (MERAV), which can be found on the world wide web at www.merav.wi.mit.edu.

Agents

In one embodiment, an agent that inhibits a SiglecF^{high} cell is administered as an anti-cancer therapy. An agent can be a small molecule, an inhibitory nucleic acid, an antibody or antigen-binding fragment thereof, or an antibody reagent, an inhibitory polypeptide, and antisense oligonucleotide, an immunotherapy, a nanoparticle, or a polymer.

An agent targets at least one gene comprised in the SiglecF^{high} cell gene profile. An agent is considered effective for inhibiting a SiglecF^{high} cell if said agent can, for example, upon contacting a SiglecF^{high} cell, inhibit the activity, level, and/or migration of the cell.

An agent can induce cell death and kill the SiglecF^{high} cells. An agent can reduce the amount of SiglecF^{high} cells in a subject compared to the amount of SiglecF^{high} cells in the subject prior to administration of said agent. Alternatively, the agent can eliminate all SiglecF^{high} cells in a subject. The agent can also inhibit and/reduce the rate at which new SiglecF^{high} cells are induced (e.g., by OCN⁺ cells). Immunofloresence detection using antibodies specific to a SiglecF^{high} cell (e.g., an antibody against a gene comprised in the gene profile of a SiglecF^{high} cell) in combination with cell death markers (e.g., Caspase) can be used to determine if cell death has occurred following administration of an agent. Alternatively, mRNA and protein levels of a given target (e.g., a gene comprised the SiglecF^{high} gene profile) can be assessed using RT-PCR and western-blotting, respectively.

An agent can put the SiglecF^{high} cell into anergy. As used herein, the term “anergy” refers to the state of an unresponsive immune system. An immune cell that is in anergy can fail to respond to a specific antigen, or perform its intended function. An agent can disrupt the functional interaction of the SiglecF^{high} cell and a tumor cell and/or tumor microenvironment. As used herein, the “functional interation” is defined as the critical interaction between the SiglecF^{high} cell and the tumor cell and/or tumor microenvironment that elicites the tumor-promoting function of the SiglecF^{high} cell. The functional interaction can be through direct binding or can be indirect (e.g., mediated through an intermediate) of a SiglecF^{high} cell and the tumor and/or tumor microenvironment. Alternatively, an agent can disrupt the tumor-promoting function of a SiglecF^{high} cell. Assays that measure tumor progression and/or growth can be used to determine if the activity of the SiglecF^{high} cell is inhibited by an agent, (e.g., biopsy and noninvasive imaging).

An agent can inhibit the physical interaction of the SiglecF^{high} cell and a tumor cell and/or tumor microenvironment. The physical interaction can be a direct binding interaction, or an indirect interaction. The physical interaction of the SiglecF^{high} cell and a tumor cell and/or tumor microenvironment can be assessed using, for example, co-immunoprecipitation assays. An agent can disrupt the migration of the SiglecF^{high} cell towards a tumor cell and/or tumor microenvironment. For example, an agent can inhibit, reduce, or slow the migration of the SiglecF^{high} cell out of, for example, the bone environment, the blood stream, or the migration into an organism comprising a tumor (e.g., the lung comprising a lung tumor). An agent can inhibit the tropism of the SiglecF^{high} cell. As used herein, “tropism” refers to the growth or turning movement of a cell (e.g., a SiglecF^{high} cell) in a particular direction in response to an external stimulus (e.g., chemical signaling from the tumor and/or tumor microenvironment.) Immunofloresence detection using antibodies specific to a SiglecF^{high} cell (e.g., an antibody against a gene comprised in the gene profile of a SiglecF^{high} cell) can be used to detect the location of a SiglecF^{high} cell in a subject.

In one embodiment, the agent that inhibits a SiglecF^{high} cell is a small molecule. A small molecule can be defined as a low molecular weight (e.g., ranging from 500 to 900 daltons) organic compound that can regulate a biological process. It is desired that the small molecule can diffuse across membranes to reach its given target (e.g., a SiglecF^{high} cell). Small molecules can bind their given target with high affinity and act as an effector upon binding. Small molecules that bind a given target are known in the art and can be determined by a skilled person. Methods for screening small molecules are known in the art and can be used to identify a small molecule that is efficient at, for example, inhibiting the tumor-promoting function of a SiglecF^{high} cell.

In some embodiments of any of the aspects, the agent that inhibits a SiglecF^{high} cell is an inhibitory nucleic acid. Inhibitors of the expression of a given gene can be an inhibitory nucleic acid. In some embodiments of any of the aspects, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of a target, e.g. a SiglecF^{high} cell. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA (e.g., a gene found within the SiglecF^{high} gene profile) level found in the cell without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

Exemplary embodiments of inhibitory nucleic acids can include, e.g., siRNA, shRNA, miRNA, and/or a miRNA, which are well known in the art.

In some embodiments of any of the aspects, the agent is siRNA that inhibits a SiglecF^{high} cell. In some embodiments of any of the aspects, the agent is shRNA that inhibits a SiglecF^{high} cell. In some embodiments of any of the aspects, the agent is miRNA that inhibits a SiglecF^{high} cell. One skilled in the art would be able to design siRNA, shRNA, or miRNA to target a SiglecF^{high} cell, e.g., using publically available design tools. siRNA, shRNA, or miRNA is commonly made using companies such as Dharmacon (Layfayette, Colo.) or Sigma Aldrich (St. Louis, Mo.). One skilled in the art will be able to readily assess whether the siRNA, shRNA, or miRNA effective target for a the SiglecF^{high} cell for its downregulation, for example by transfecting the siRNA, shRNA, or miRNA into cells and detecting the levels of a gene found within the SiglecF^{high} cell gene profile via western-blotting.

In one embodiment, the agent that inhibits a SiglecF^{high} cell is an antibody or antigen-binding fragment thereof, or an antibody reagent. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

In one embodiment, the agent that inhibits a SiglecFhigh cell is an inhibitory polypeptide. The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g. an inhibitory polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

In one embodiment, the agent that inhibits a SiglecF^{high} cell is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to a gene comprised in the SiglecF^{high} gene profile. Thus, oligonucleotides are chosen that are sufficiently complementary to the target, i.e., that hybridize sufficiently well and with sufficient specificity, to give the desired effect.

In one embodiment, the agent that inhibits a SiglecF^{high} cell is an immunotherapy. As used herein, an “immunotherapy” refers to a treatment that induces, enhances, or suppresses an immune response. Immunotherapies can be used to induce an immune system to target a cell (e.g., modified T cell that is directed to find and a SiglecF^{high} cell). In one embodiment, a CAR T cell is used to target SiglecF^{high} cells for cell death. The terms “chimeric antigen receptor” or “CAR” as used herein refer to engineered T cell receptors, which graft a ligand or antigen specificity onto T cells (for example naïve T cells, central memory T cells, effector memory T cells or combinations thereof). CARs are also known as artificial T-cell receptors, chimeric T-cell receptors or chimeric immunoreceptors.

A CAR places a chimeric extracellular target-binding domain that specifically binds a target, e.g., a polypeptide expressed on the surface of a cell (e.g., a SiglecF^{high} cell) to be targeted for a T cell response onto a construct including a transmembrane domain, and intracellular domain(s) (including signaling domains) of a T cell receptor molecule. In one embodiment, the chimeric extracellular target-binding domain comprises the antigen-binding domain(s) of an antibody that specifically binds an antigen expressed on a cell to be targeted for a T cell response (e.g., a SiglecF^{high} cell). The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

As used herein, a “CAR T cell” or “CAR-T” refers to a T cell which expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

In one embodiment, the agent that inhibits a SiglecF^{high} cell is a nanoparticle. In some embodiments, inhibitory nucleic acid, a small molecule, an antibody or antigen-binding fragment thereof, antibody reagent, inhibitory polypeptide, antisense oligonucleotide, or immunotherapy can be can be present in and/or on a nanoparticle. As used herein, the term “nanoparticle” refers to particles that are on the order of about 10-9 or one to several billionths of a meter. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle.

In one embodiment, the agent that inhibits a SiglecF^{high} cell is a polymer. Compositions of polymers and methods for generating said polymers are well known in the art.

Cancer

“Cancer” is a hyperproliferation of cells that have lost normal cellular control, resulting in unregulated growth, lack of differentiation, local tissue invasion, and metastasis. Cancers are classified based on the histological type (e.g., the tissue in which they originate) and their primary site (e.g., the location of the body the cancer first develops). There are 6 major histological types of cancer: carcinoma, sarcoma, myeloma, leukemia, lymphoma, and mixed types (cancer that comprises various components within one histological type, or from two or more histological types).

A carcinoma is a cancer that originates in an epithelial tissue. Carcinomas account for approximately 80-90% of all cancers. Carcinomas can affect organs or glands capable of secretion (e.g., breasts, lung, prostate, colon, or bladder). There are two subtypes of carcinomas: adenocarcinoma, which develops in an organ or gland, and squamous cell carcinoma, which originates in the squamous epithelium. Adenocarcinomas generally occur in mucus membranes, and are observed as a thickened plaque-like white mucosa. They often spread easily through the soft tissue where they occur. Squamous cell carcinomas can originate from any region of the body. Examples of carcinomas include, but are not limited to, prostate cancer, colorectal cancer, microsatellite stable colon cancer, microsatellite instable colon cancer, hepatocellular carcinoma, breast cancer, lung cancer, small cell lung cancer, non-small cell lung cancer, lung adenocarcinoma, melanoma, basal cell carcinoma, squamous cell carcinoma, renal cell carcinoma, ductal carcinoma in situ, invasive ductal carcinoma.

Sarcomas are cancers that originate in supportive and connective tissues, for example bones, tendons, cartilage, muscle, and fat. Sarcoma tumors usually resemble the tissue in which they grow. Non-limiting examples of sarcomas include, Osteosarcoma or osteogenic sarcoma (originating from bone), Chondrosarcoma (originating from cartilage), Leiomyosarcoma (originating from smooth muscle), Rhabdomyosarcoma (originating from skeletal muscle), Mesothelial sarcoma or mesothelioma (originate from membranous lining of body cavities), Fibrosarcoma (originating from fibrous tissue), Angiosarcoma or hemangioendothelioma (originating from blood vessels), Liposarcoma (originating from adipose tissue), Glioma or astrocytoma (originating from neurogenic connective tissue found in the brain), Myxosarcoma (originating from primitive embryonic connective tissue), or Mesenchymous or mixed mesodermal tumor (originating from mixed connective tissue types).

Myelomas are cancers that originate in plasma cells of bone marrow. Non-limiting examples of myelomas include multiple myeloma, plasmacytoma and amyloidosis.

Leukemias (also known as “blood cancers”) are cancers of the bone marrow, which is the site of blood cell production. Leukemia is often associated with the overproduction of immature white blood cells. Immature white blood cells do not function properly, rendering the patient prone to infection. Leukemia additionally affects red blood cells, and can cause poor blood clotting and fatigue due to anemia. Leukemia can be classified as being acute myeloid leukemia (AML), Chronic myeloid leukemia (CML), Acute lymphocytic leukemia (ALL), and Chronic lymphocytic leukemia (CLL). Examples of leukemia include, but are not limited to, Myelogenous or granulocytic leukemia (malignancy of the myeloid and granulocytic white blood cell series), Lymphatic, lymphocytic, or lymphoblastic leukemia (malignancy of the lymphoid and lymphocytic blood cell series), and Polycythemia vera or erythremia (malignancy of various blood cell products, but with red cells predominating).

Lymphomas develop in the glands or nodes of the lymphatic system (e.g., the spleen, tonsils, and thymus), which purifies bodily fluids and produces white blood cells, or lymphocytes. Unlike leukemia, lymphomas form solid tumors. Lymphoma can also occur in specific organs, for example the stomach, breast, or brain; this is referred to as extranodal lymphomas). Lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma. Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma.

In one embodiment, the cancer is lung cancer. Exemplary lung cancers include non-small cell lung cancer, small cell lung cancer, sall cell carcinoma, combined small cell carcinoma, lung carcinoid tumor, adenocarcinoma, squamous cell carcinoma, or large cell carcinoma. In one embodiment, the cancer is non-small cell lung cancer. In another embodiment, the cancer is KRAS⁺ non-small cell lung cancer.

In one embodiment, the cancer is a solid tumor. Non-limiting examples of solid tumors include Adrenocortical Tumor, Alveolar Soft Part Sarcoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carinomas.

In one in embodiment, the cancer is metastatic.

It is contemplated herein that an agent that inhibits a SiglecF^{high} cell can be used to treat all cancers, and should not be limited to the cancer types listed in this present specification.

Administration

In some embodiments, the methods described herein relate to treating a subject having or diagnosed as having cancer comprising administering an agent that inhibits a SiglecF^{high} cell as described herein. Subjects having a condition can be identified by a physician using current methods of diagnosing a condition (e.g., cancer). Symptoms and/or complications of the condition, which characterize this disease and aid in diagnosis are well known in the art and include but are not limited to, fatigue, persistent cough with blood in sputum, and pain in chest. Tests that may aid in a diagnosis of, e.g. the condition, include but are not limited to, needle aspiration biopsy, and sputum analysis, and are known in the art for a given condition. A family history for a condition, or exposure to risk factors for a condition can also aid in determining if a subject is likely to have the condition or in making a diagnosis of the condition.

The agents described herein can be administered to a subject having or diagnosed as having a cancer (e.g., non-small cell lung cancer). In some embodiments, the methods described herein comprise administering an effective amount of an agent that inhibits a SiglecF^{high} cell to a subject in order to alleviate a symptom of the cancer. As used herein, “alleviating a symptom of the cancer” is ameliorating any condition or symptom associated with cancer. As compared with an equivalent untreated control, such reduction is by at least 5%, 10%, 20%, 40%, 50%, 60%, 80%, 90%, 95%, 99% or more as measured by any standard technique. A variety of means for administering the agent that inhibits a SiglecF^{high} cell described herein to subjects are known to those of skill in the art. In one embodiment, the agent that inhibits a SiglecF^{high} cell is administered systemically or locally. In one embodiment, the agent that inhibits a SiglecF^{high} cell is administered intravenously. In another embodiment, the agent that inhibits a SiglecF^{high} cell is administered at the site of the tumor. The route of administration of an agent that inhibits a SiglecF^{high} cell will be optimized for the type of agent being delivered, and can be determined by a skilled person.

The term “effective amount” as used herein refers to the amount of an agent needed to alleviate at least one or more symptom of the cancer. The term “therapeutically effective amount” therefore refers to an amount of an agent that is sufficient to provide a particular anti-cancer effect when administered to a typical subject. An effective amount as used herein, in various contexts, would also include an amount of an agent sufficient to delay the development of a symptom of the cancer, alter the course of a symptom cancer (for example but not limited to, slowing the progression of a cancer), or reverse a symptom of the cancer. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., needle aspiration biospy, among others. The dosage can be determined by a physician and adjusted, as necessary, to suit observed effects of the treatment.

Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Combination Therapy

In one embodiment, the agent that inhibits a SiglecF^{high} cell is administered as a monotherapy. In one embodiment, the agent that inhibits a SiglecF^{high} cell is administered in combination with a chemotherapeutic agent, an anti-tumor agent, radiation, or surgery. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (1R,2R,45)-4-[(2R)-2 [(1R,95,125,15R,16E,18R,19R,21R,235,24E,26E,28Z,305,325,35R)-1,18-dihydroxy-19,30-dimethoxy-15,17,21,23,29,35-hexamethyl-2,3,10,14,20-pentaoxo-11,36-dioxa-4-azatricyclo[30.3.1.04′9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOl); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-(SEQ ID NO: 84), inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2-yl)amino)-1-oxo-3-phenylpropan-2-yl)-2-((5,)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(llS′)-2-[(2R)-2-methyl-2-oxiranyl]-2-oxo-1-(phenylmethyl)ethyl]-L-serinamide (ONX-0912).

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloffs Clinical Oncology, 2013 Elsevier; and Fischer D S (ed): The Cancer Chemotherapy Handbook, 4th ed. St. Louis, Mosby-Year Book, 2003).

In on embodiment, the agent that inhibits a SiglecF^{high} cell is administered in combination with a checkpoint inhibitor. A checkpoint inhibitor can be a small molecule, inhibitory RNA/RNAi molecule (both single and double stranded), an antibody, antibody reagent, or an antigen-binding fragment thereof that specifically binds to at least one immune checkpoint protein. Common checkpoints that are targeted for therapeutics include, but are not limited to PD-1, CTLA4, TIM3, LAG3 and PD-L1. Inhibitors of their checkpoint regulators are known in the art.

Non-limiting examples of checkpoint inhibitors (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb) atezolizumab (PD-L1; Genentech); galiximab (B7.1; Biogen); IMP321 (LAG3: Immuntep); BMS-986016 (LAG3; Bristol Meyers Squibb); SMB-663513 (CD137; Bristol-Meyers Squibb); PF-05082566 (CD137; Pfizer); IPH2101 (KIR; Innate Pharma); KW-0761 (CCR4; Kyowa Kirin); CDX-1127 (CD27; CellDex); MEDI-6769 (Ox40; MedImmune); CP-870,893 (CD40; Genentech); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); TSR-022 (TIM3; Tesaro).

In one embodiment, a second therapeutic is the administration of radiation treatment or surgery to remove all or part of a tumor and optionally, the surrounding tissue. Radiation and surgery for treatment of cancer are known in the art, and can be administered and/or performed by a skilled persion.

In one embodiment, administration of an agent that inhibits a SiglecF^{high} cell is administered prior to administration of a second therapeutic. In one embodiment, administration of the agent is administered following to administration of a second therapeutic. Administration of the agent and the second therapeutic can be done at different time points, or at substantially the same time. An agent that that inhibits a SiglecF^{high} cell can be comprised within a composition comprising a second therapeutic (e.g., comprised in a composition comprising a chemotherapeutic agent).

Parenteral Dosage Forms

Parenteral dosage forms of an agent inhibit SiglecF^{high} cells also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol Formulations

Where therapeutically indicated, an agent that inhibits SiglecF^{high} cells can be packaged in a pressurized aerosol container together with suitable propellants, for example, hydrocarbon propellants like propane, butane, or isobutane with conventional adjuvants. An agent inhibit SiglecF^{high} cells can also be administered in a non-pressurized form such as in a nebulizer or atomizer. An agent inhibit SiglecF^{high} cells can also be administered directly to the airways in the form of a dry powder, for example, by use of an inhaler.

Suitable powder compositions include, by way of illustration, powdered preparations of an agent inhibit SiglecF^{high} cells can be thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of an agent that inhibits SiglecF^{high} cells can further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms

In some embodiments of the aspects described herein, an agent that inhibits SiglecF^{high} cells can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent inhibit SiglecF^{high} cells is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with an agent inhibit SiglecF^{high} cells. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

Efficacy

The efficacy of an agent that inhibits SiglecF^{high} cells, e.g., for the treatment of a cancer described herein, can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., tumor size or inflammatory cytokine levels. Efficacy can also be measured by a failure of an individual to worsen as assessed by hospitalization, or need for medical interventions (i.e., progression of the cancer is halted). Methods of measuring these indicators are known to those of skill in the art and/or are described herein.

Efficacy can be assessed in animal models of a condition described herein, for example, a mouse model of cancer, a pathogenic infection model, or an appropriate animal model of autoimmune or inflammatory disease, as the case may be. When using an experimental animal model, efficacy of treatment is evidenced when a statistically significant change in a marker is observed, e.g. decrease in tumor size or change in inflammatory cytokines.

Effective treatment can include, but is not limited to, a reduction in tumor size, a reduction the rate of tumor growth, and/or a reduction in metastasis (for example, a reduction of metastatic nodules).

Some embodiments of the technology described herein can be defined according to any of the following numbered paragraphs:
1) A method for treating cancer, the method comprising: administering an agent that inhibits the activity, level, and/or migration of a SiglecF^{high} cell.
2) A method for treating cancer, the method comprising: identifying a population of SiglecF^{high} cells in a patient; and administering an agent to the patient that inhibits the activity, level, and/or migration of said population of SiglecF^{high} cells.
3) The method of paragraphs 1 and 2, wherein the cancer is lung cancer.
4) The method of paragraph 3, wherein the lung cancer is non-small cell lung cancer.
5) The method of paragraph 4, wherein the is non-small cell lung cancer is KRAS⁺ non-small cell lung cancer.
6) The method of paragraph 3, wherein the lung cancer is selected from the group consisting of: small cell lung cancer, small cell carcinoma, combined small cell carcinoma, lung carcinoid tumor, adenocarcinoma, squamous cell carcinoma, or large cell carcinoma.
7) The method of paragraphs 1 and 2, wherein the cancer is a solid tumor cancer.
8) The method of paragraphs 1 and 2, wherein the agent is a small molecule.
9) The method of paragraphs 1 and 2, wherein the agent is an inhibitory nucleic acid.
10) The method of paragraphs 1 and 2, wherein the agent is an antibody or antigen-binding fragment thereof, or antibody reagent.
11) The method of paragraphs 1 and 2, wherein the agent is an inhibitory polypeptide.
12) The method of paragraphs 1 and 2, wherein the agent is an antisense oligonucleotide.
13) The method of paragraphs 1 and 2, wherein the agent is an immunotherapy.
14) The method of paragraphs 1 and 2, wherein the agent is a nanoparticle.
15) The method of paragraphs 1 and 2, wherein the agent is a polymer.
16) The method of paragraphs 1 and 2, wherein the inhibiting the level kills the SiglecF^{high} cell.
17) The method of paragraphs 1 and 2, wherein the inhibiting the level inhibits the rate at which the SiglecF^{high} cell is induced.
18) The method of paragraphs 1 and 2, wherein the inhibiting the activity puts the cell into anergy.
19) The method of paragraphs 1 and 2, wherein the inhibiting the activity disrupts the functional interaction of the SiglecF^{high} cell and a tumor cell.
20) The method of paragraphs 1 and 2, wherein the inhibiting the activity disrupts the functional interaction of the SiglecF^{high} cell and a tumor microenvironment.
21) The method of paragraphs 1 and 2, wherein the inhibiting the activity disrupts the tumor-promoting function of a SiglecF^{high} cell.
22) The method of paragraphs 1 and 2, wherein the inhibiting the migration disrupts the physical interaction of the SiglecF^{high} cell and a tumor cell.
23) The method of paragraphs 1 and 2, wherein the inhibiting the migration disrupts the physical interaction of the SiglecF^{high} cell and a tumor microenvironment.
24) The method of paragraphs 1 and 2, wherein the inhibiting the migration disrupts the movement of the SiglecF^{high} cell towards a tumor cell.
25) The method of paragraphs 1 and 2, wherein the inhibiting the migration disrupts the movement of the SiglecF^{high} cell towards a tumor microenvironment.
26) The method of paragraphs 1 and 2, wherein the inhibiting the migration inhibits the tropism of the SiglecF^{high} cell.
27) The method of paragraph 2, wherein the identifying a population of SiglecF^{high} cells comprises: assessing the gene profile of a sample obtained from said patient; and comparing it to the gene profile of SiglecF^{high} cells.
28) The method of paragraph 27, wherein the sample is obtained from a biopsy of a lung.
29) The method of paragraph 27, wherein the sample is obtained from a biopsy of a lymph node.
30) The method of paragraph 27, wherein the sample is obtained from bronchoalveolar lavage.
31) The method of paragraphs 1 and 2, further administering a second therapeutic.
32) The method of paragraph 31, wherein the second therapeutic is a chemotherapeutic, an anti-tumor agent, radiation, or surgery.
33) A method of treating non-small cell lung cancer, the method comprising: administering an agent that inhibits the activity, level, and/or migration a SiglecF^{high} cell.
34) The method of paragraph 33, further administering a second therapeutic.
35) The method of paragraph 33, further comprising, before administering an agent, in a patient identifying a population of SiglecF^{high} cells.
36) The method of paragraph 33, wherein the identifying a population of SiglecF^{high} cells comprises: assessing the gene profile of a sample obtained from said patient; and comparing it to the gene profile of SiglecF^{high} cells.
37) The method of paragraph 36, wherein the sample is obtained from a biopsy of a lung.
38) The method of paragraph 36, wherein the sample is obtained from a biopsy of a lymph node.
39) The method of paragraph 36, wherein the sample is obtained from bronchoalveolar lavage.

### EXAMPLES

### Example 1

Bone marrow-derived myeloid cells can accumulate in high numbers within solid tumors and mediate functions that foster cancer outgrowth. Immune-neoplastic interactions in the local tumor microenvironment have been intensively investigated, but the contribution of the host systemic environment to tumor growth remains poorly understood. Described herein is experimental data showing in mice and in cancer patients (n=70) that lung adenocarcinomas increase bone stromal activity even in the absence of local metastasis. Animal studies described herein further reveal that the cancer-induced bone phenotype involves bone-resident osteocalcin-expressing (Ocn⁺) osteoblastic cells. These cells promote cancer by remotely supplying a distinct subset of tumor-infiltrating SiglecF^{high} neutrophils, which exhibit discrete cancer-promoting properties. Experimentally-induced reduction in the number of Ocn⁺ cells suppresses both the neutrophil response and lung tumor outgrowth. These observations reveal a surprising role for osteoblasts as remote regulators of lung cancer and identify SiglecF^{high} neutrophils as myeloid cell effectors of the osteoblast-driven pro-tumoral response.

### Example 2

### Results

Lung Tumors Modulate Bones in Mice and Patients.

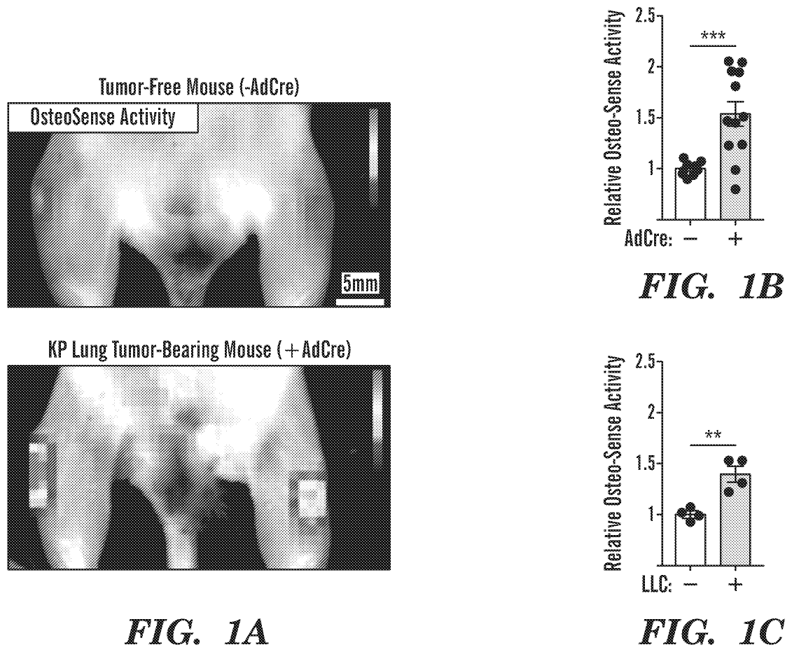

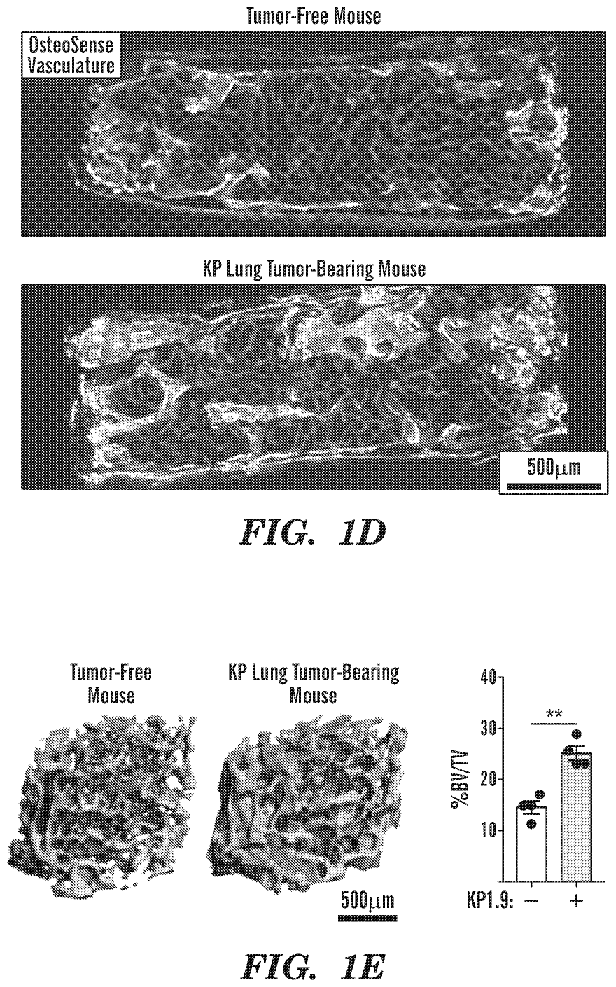

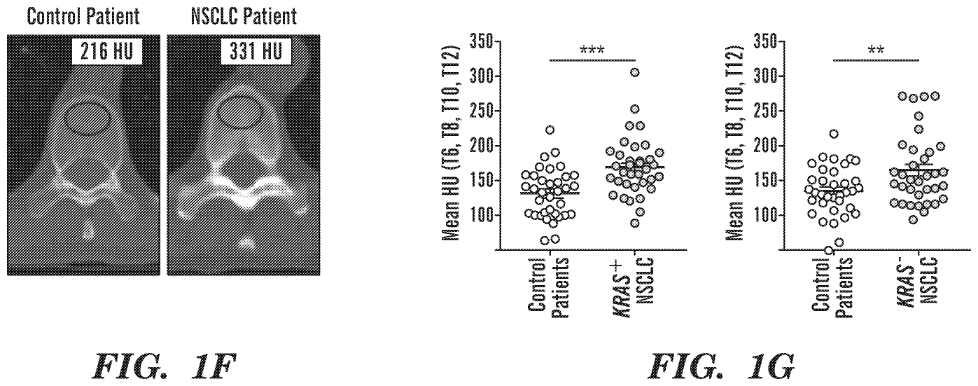

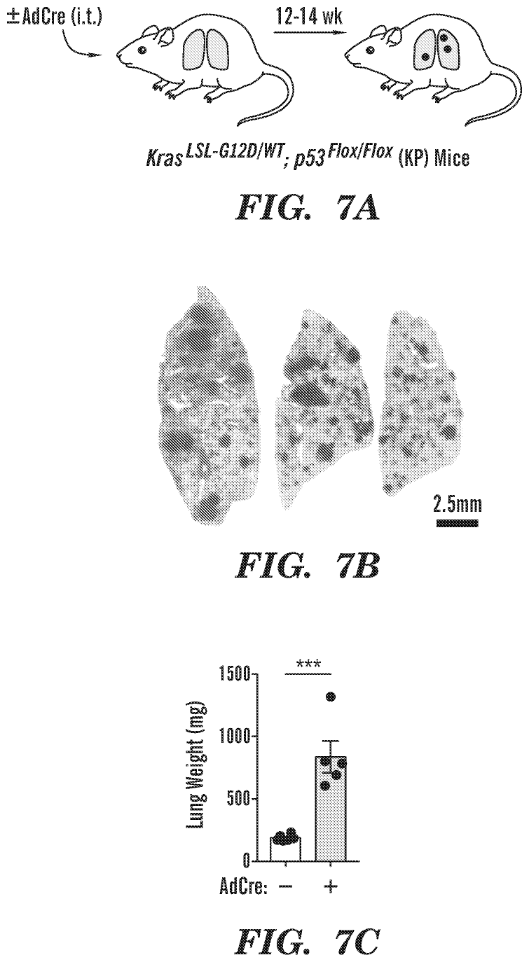

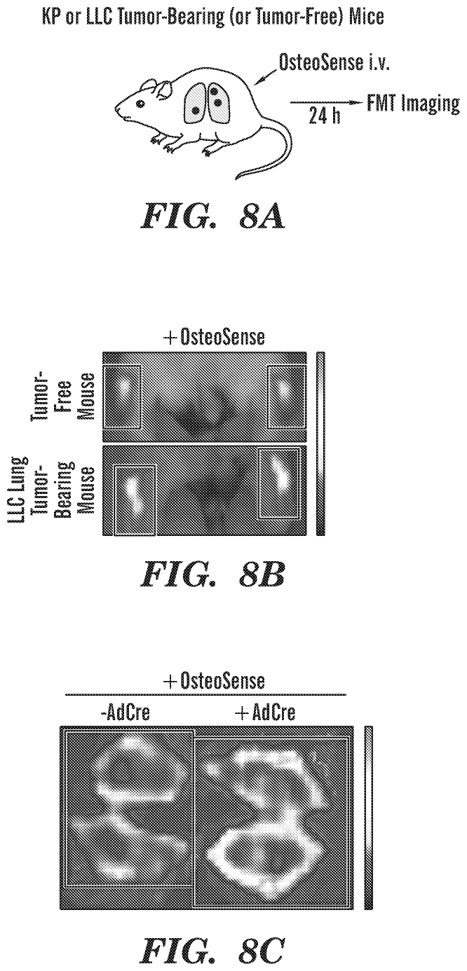

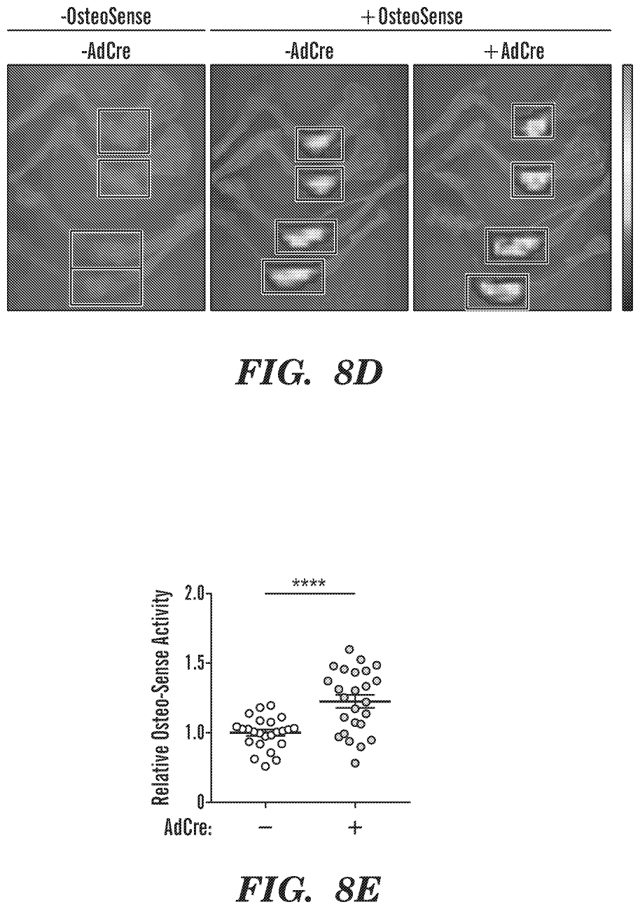

To test whether lung tumors disrupt bone homeostatic activity, a fluorescent bisphosphonate derivative (OsteoSense-750EX) (18) that binds hydroxyapatite minerals in areas of active bone formation and is detectable in vivo by fluorescence-mediated tomography (FMT) was initially used (19). The use of a mouse model of lung adenocarcinoma in which tumors are induced by intratracheal delivery of Adenovirus-Cre, which activates oncogenic Kras and deletes the tumor suppressor Trp53 (hereafter referred to as KP; FIG. 7A-7C), and whose growth recapitulates key aspects of the human disease was additionally considered (20). Additionally, the following cell lines were used: the KP1.9 tumor cell line, which derives from KP lung tumor nodules and behaves similarly to its autochthonous counterpart (21), and the Lewis Lung Carcinoma (LLC) cell line, a commonly used murine lung tumor model. In vivo FMT analysis of the femoral-tibial joint (FIG. 8A) showed significantly elevated OsteoSense activity in both KP (FIGS. 1A and B) and LLC (FIGS. 1C and 8B) lung tumor-bearing mice, when compared to tumor-free controls.

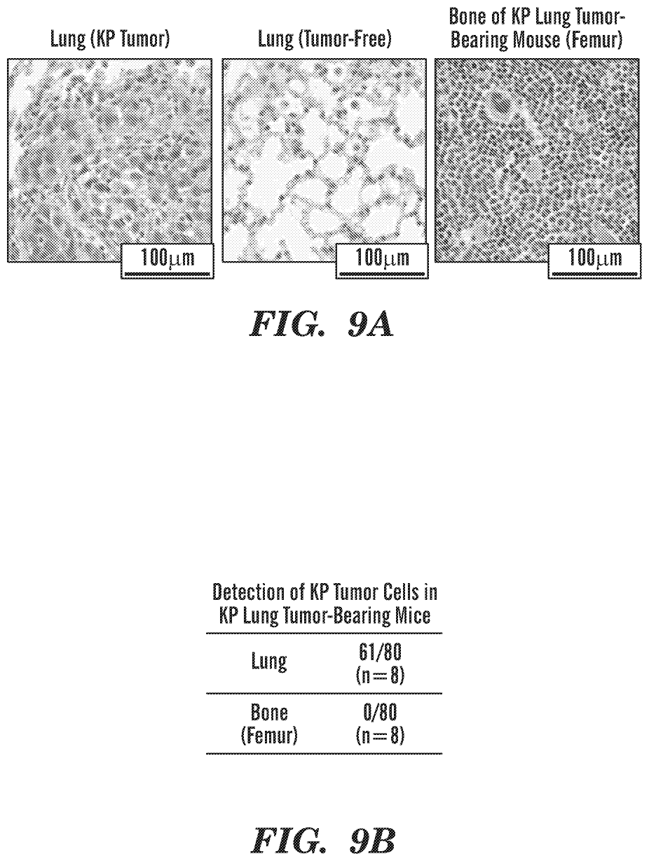

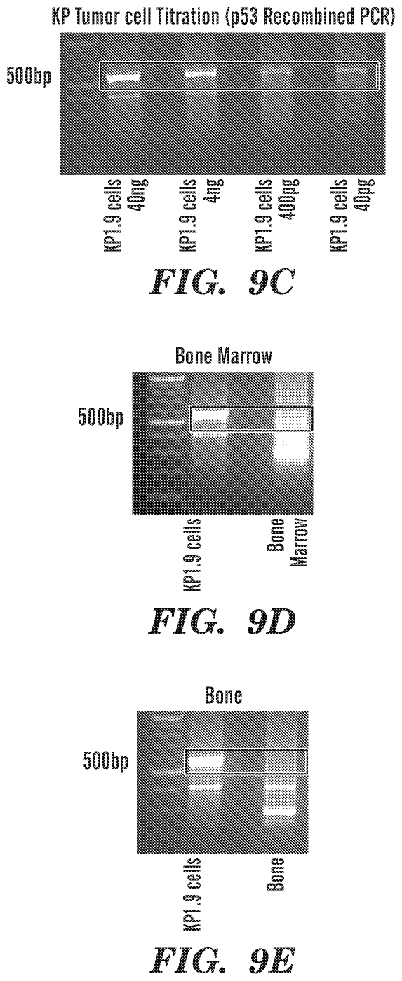

Ex vivo analysis of explanted bones from KP tumor-bearing mice further revealed that this activity extended across all compartments analyzed, including the elbow joint, sternum, ribs, vertebrae and pelvic bone (FIG. 8C-8E). Bone metastases have not been reported for mice bearing KP tumors (20), which was confirmed by histology and PCR-based methods (FIG. 9A-9E). These studies indicate that lung tumors can disrupt bone stromal activity in absence of local metastasis.

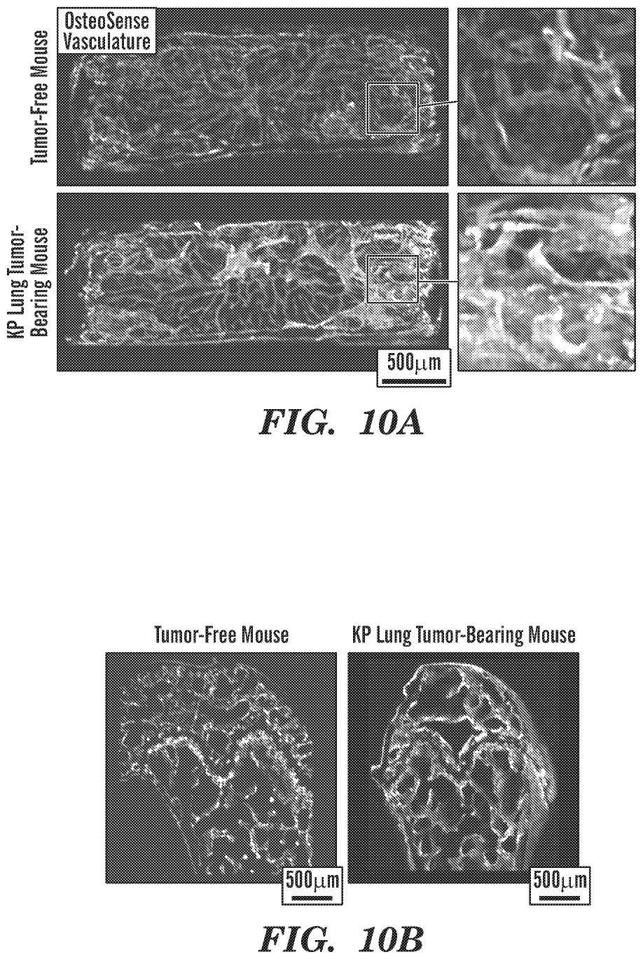

By using confocal microscopy, increased OsteoSense signal in the sternum (FIG. 1D and FIG. 10A) and distal femur (FIG. 10B) of KP1.9 tumor-bearing mice was found. The signal's location was separate from vasculature and enriched in areas of active bone remodeling, including the edges of sternebrae, which are associated with areas of increased bone in the sternum (FIG. 1D and FIG. 10A). OsteoSense signal was also found in the metaphysis of the femur, which is an area of active trabecular bone formation adjacent to the growth plate (FIG. 10B) as well as the epiphysis and diaphysis of the femur (FIG. 10B).

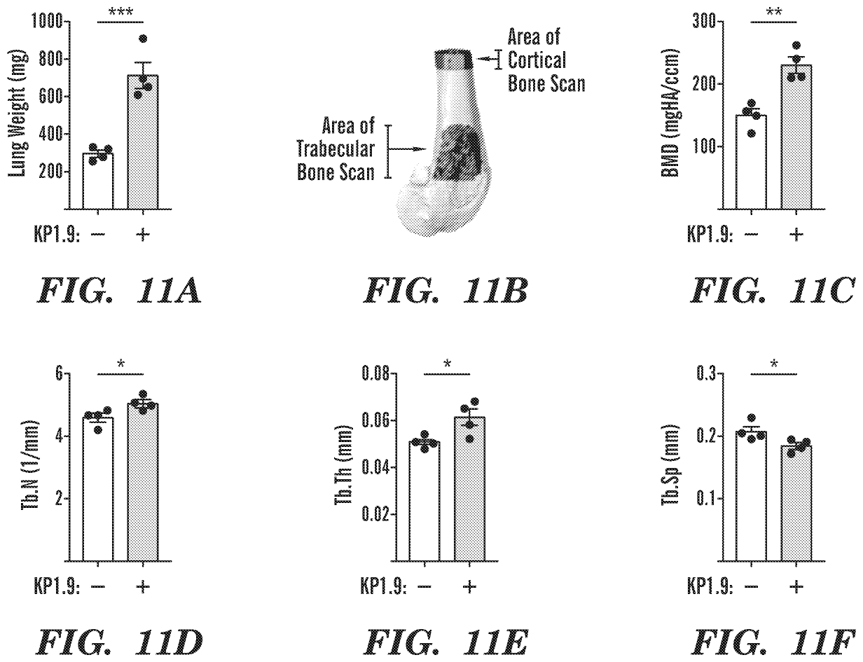

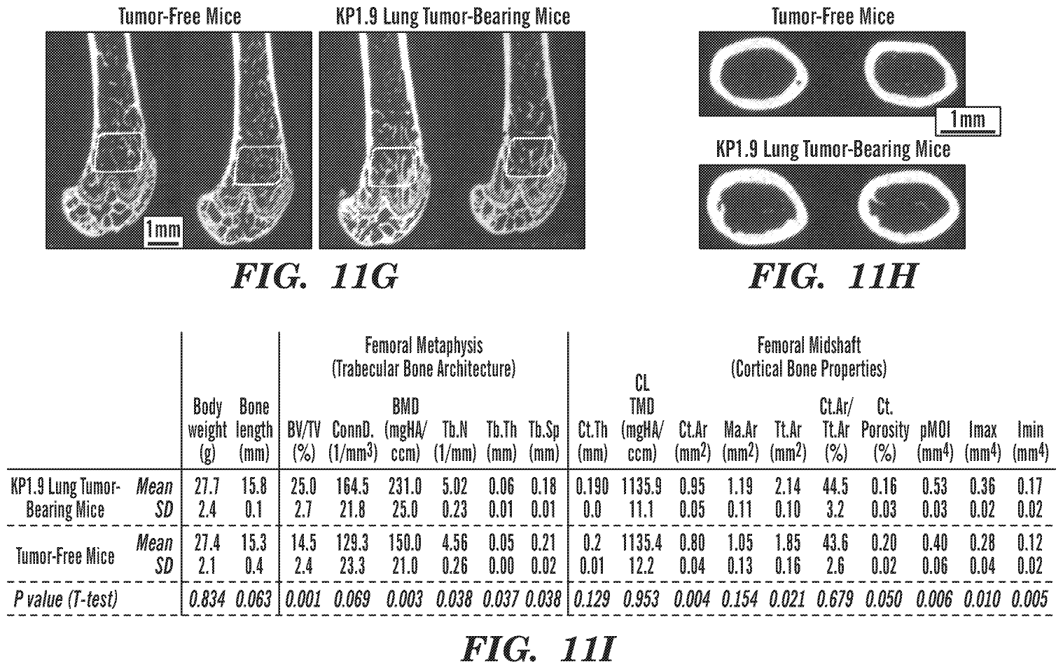

These data indicated that KP tumors can influence bone microarchitecture. Accordingly, high-resolution CT showed increased trabecular bone volume (FIG. 1E and FIG. 11A-11C) and higher mineral density of distal femoral metaphysis (FIG. 11D) in mice bearing KP tumors. The same mice also showed more (FIG. 11E) and thicker (FIG. 11F) trabeculae and decreased space between those trabeculae (FIG. 11G). Cortical bone morphology in the femoral mid-diaphysis showed similar tumor-induced phenotypes (FIG. 11H). FIG. 11I includes a complete tabulation of the CT results.

To investigate the relevance of the findings described above to human disease, 35 KRAS⁺ non-small cell lung cancer (NSCLC) patients who had undergone non-contrast chest CT prior to cancer therapy and did not have osseous metastases were observed. These patients were matched by age, sex, body mass index, and smoking status to 35 control individuals who did not have active malignancy or chronic illness and who did not use medications known to affect bone metabolism (data not shown). This analysis revealed significantly higher trabecular bone density in the thoracic vertebrae of KRA⁺ NSCLC patients compared to controls (FIG. 1F and data not shown). Similarly, equivalent analysis of 35 KRAS non-mutant NSCLC patients revealed increased bone density compared to their matched controls (FIG. 1G). Thus, lung tumor-induced changes in bone occur in both mice and humans.

Lung Tumor-Induced Bone Alterations Involve Osteoblasts.

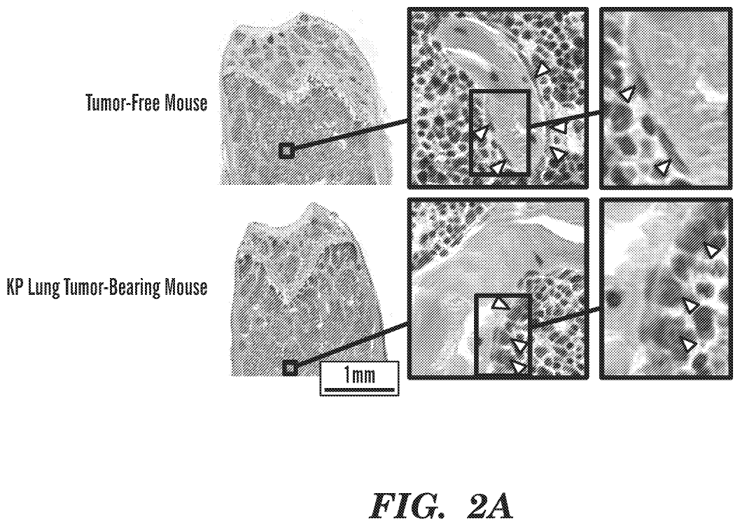

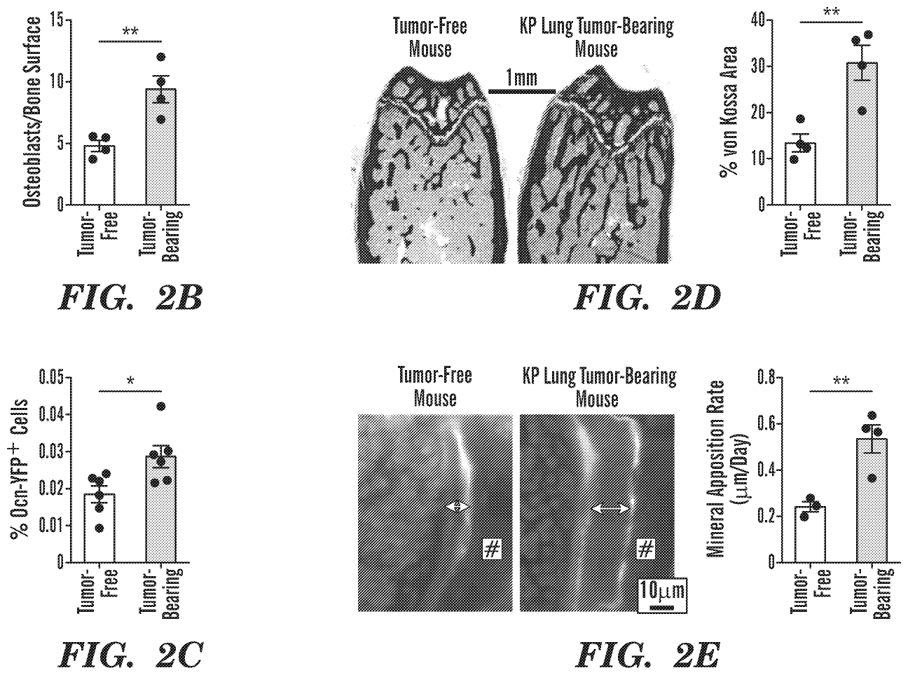

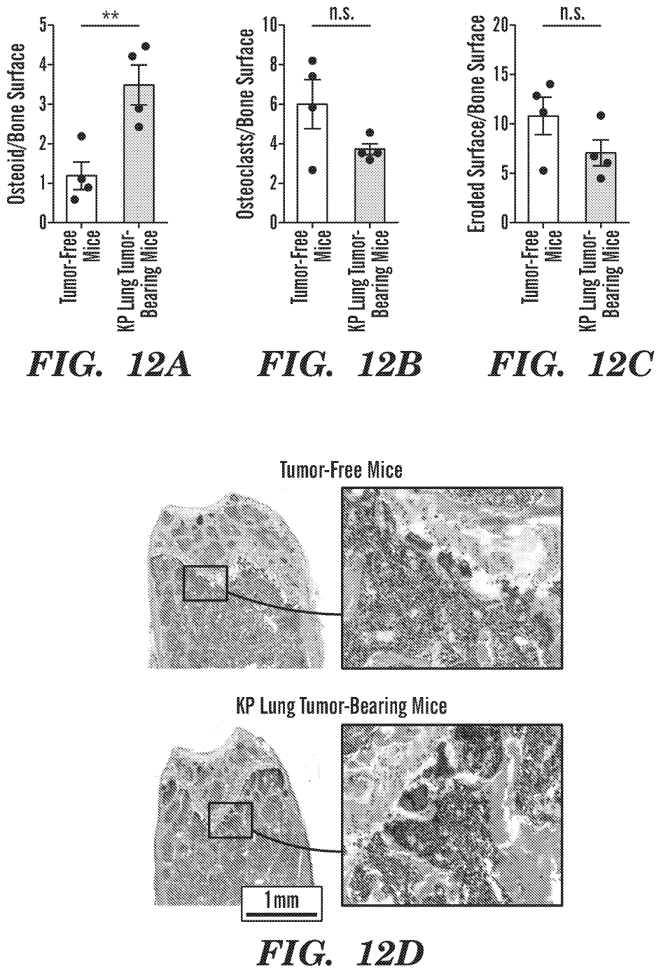

The bone phenotypes can be explained by altered osteoblast and/or osteoclast activity (22). Histological analysis of these cell lineages in the distal femur identified more osteoblasts in KP tumor-bearing mice than in tumor-free controls (FIGS. 2A and B). Osteoblasts in tumor-bearing mice also exhibited features of increased activity, including cuboidal shape and association with osteoids (FIG. 2A). Accordingly, the osteoid surface, characterizing newly formed bone, expanded in tumor-bearing mice (FIG. 12A). By contrast, osteoclast numbers (FIG. 12B) and eroded bone surfaces (FIG. 12C) were not significantly different in tumor-bearing mice, although there was a trend for lower osteoclast-related indices compared to tumor-free controls.

To trace osteoblastic lineage cells by genetic means, mice that expressed Cre-driven yellow fluorescence protein (YFP) under the control of the osteoblastic cell reporter osteocalcin (Ocn) were generated. Ocn is mainly expressed by mature osteoblasts and constitutes the major non-collagenous protein in the bone (23, 24). It was found that Ocn-YFP⁺ cells expanded in KP tumor-bearing mice when compared to tumor-free controls (FIG. 2C). Femurs of tumor-bearing mice showed increased mineralized bone and bone formation, as assessed by von Kossa staining (FIG. 2D) and dynamic histomorphometry (FIGS. 2E and 13).

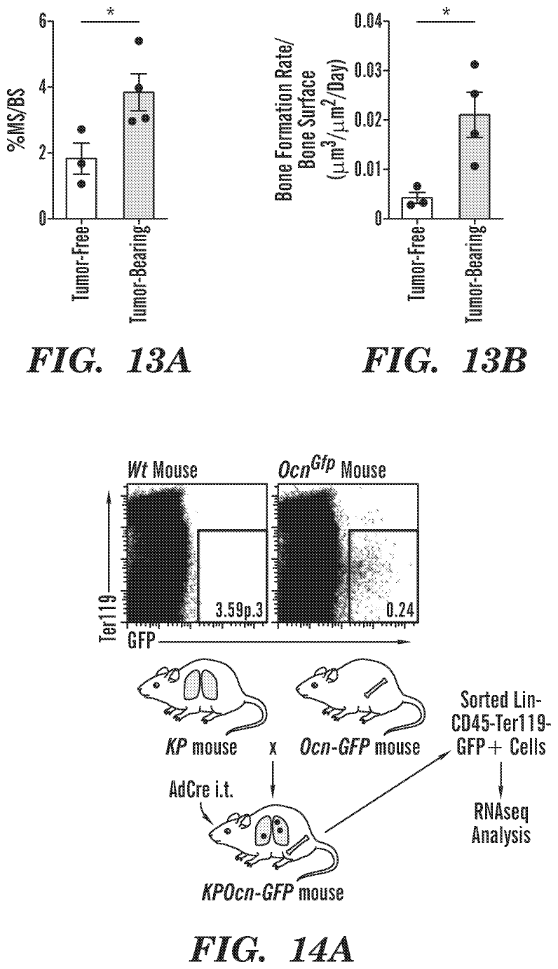

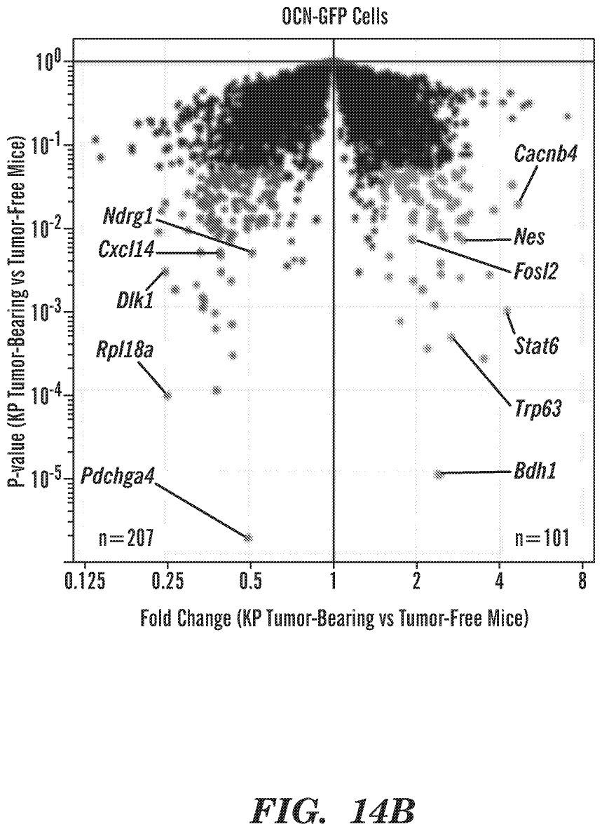

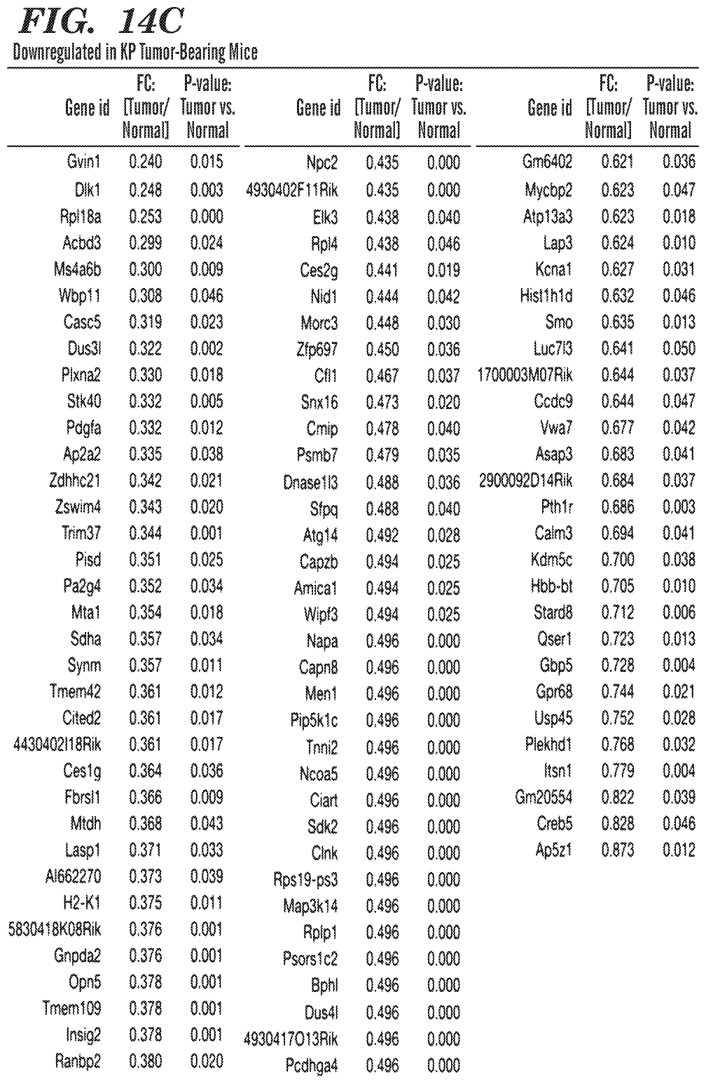

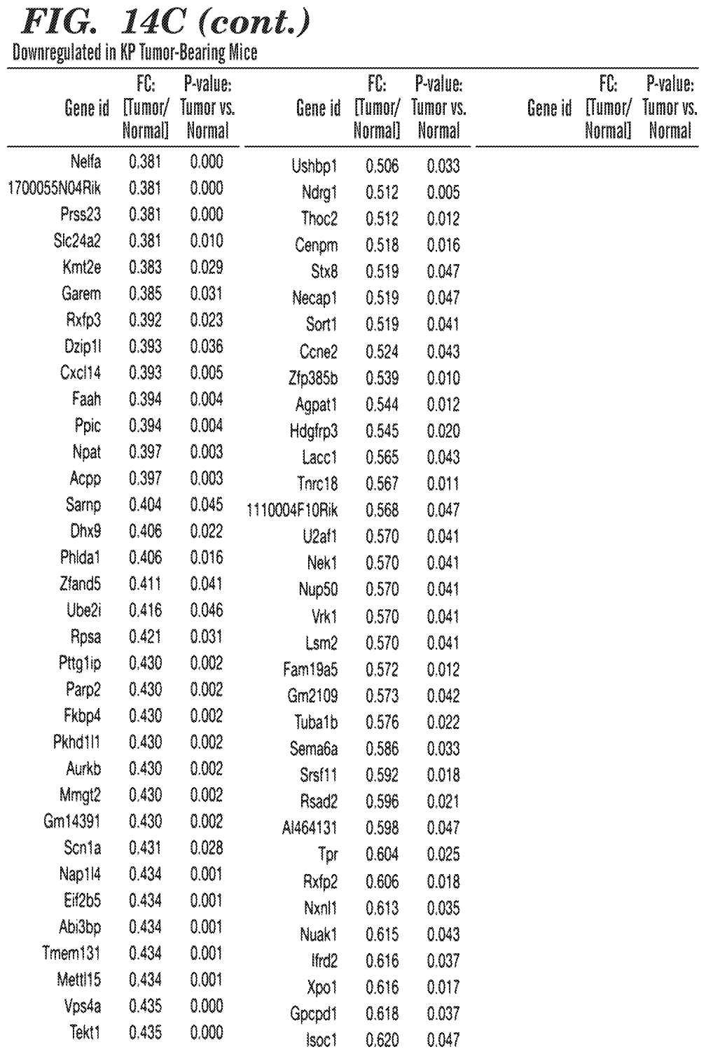

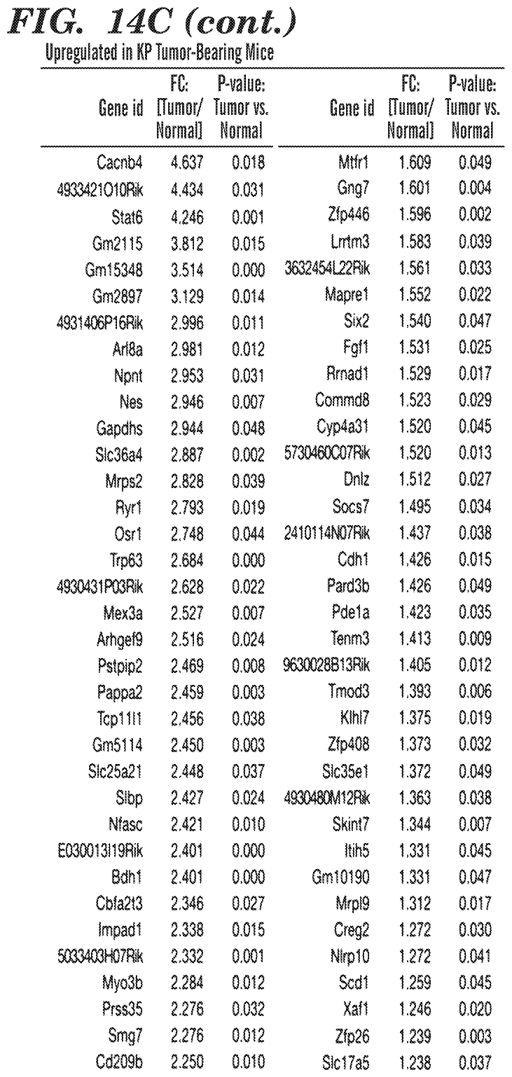

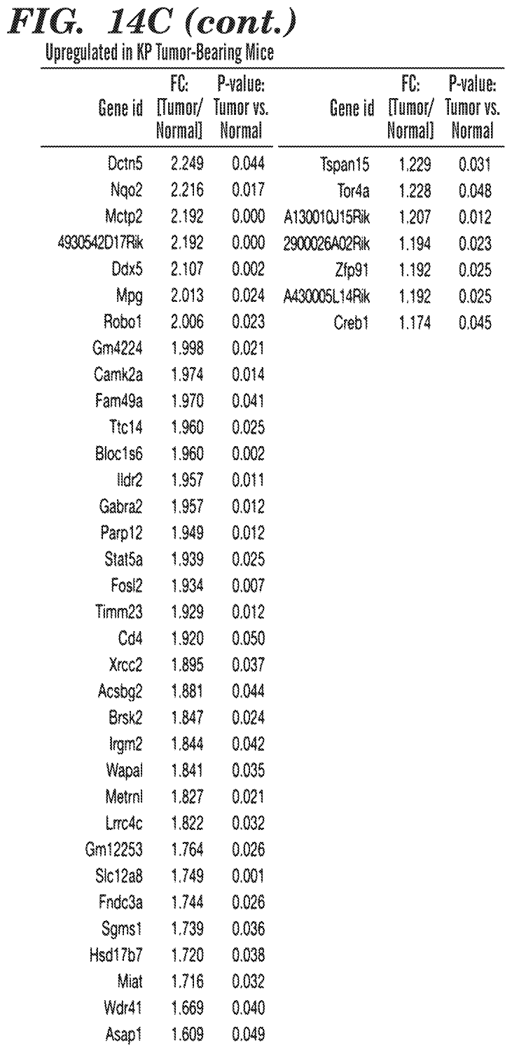

The transcriptome of Ocn⁺ cells from tumor-free and lung cancer-bearing mice were compared to assess whether Ocn⁺ cells in lung cancer-bearing mice acquire unique phenotypes. Specifically, triple transgenic KP-Ocn-GFP reporter mice (i.e., genetically engineered KP mice in which GFP expression is driven by the Ocn promoter) (20, 25) were generated, Lin⁻ CD45⁻ Ter119⁻ CD31⁻ GFP⁺ (Ocn⁺) cells were sorted from the triple transgenic mice with or without tumors, and these sorted cells were subjected to RNAseq analysis (FIG. 14A). Control experiments showed that the GFP⁺ cells expressed 1000-fold higher levels of Ocn, Osteopontin, Runx2 and other osteoblast-associated genes, when compared to CD45-Ter119⁻ CD31⁻ GFP⁻ cells from the same mice, and thus were highly enriched for Ocn-expressing osteoblasts. RNAseq analysis identified distinct changes in Ocn⁺ cells from tumor-bearing mice (101 and 207 genes were significantly upregulated and downregulated, respectively. Some of these genes were associated with bone phenotypes (FIGS. 14B and 14C)). For example, Ocn⁺ cells in tumor-bearing mice upregulated Fos12, whose overexpression leads to increased trabecular bone mass (26, 27). Decreased expression of genes, such as Dlk1 (28) and Ndrg1 (29), with ascribed bone inhibitory functions, could also be relevant for the tumor-induced bone activity. Based on the combined data presented herein, it was concluded that KP tumors increase osteoblast numbers, bone formation, and bone mineralization in vivo and induce discrete changes in Ocn⁺ cell expression of genes related to bone phenotypes.

Ocn⁺ Osteoblasts Foster Lung Tumor Growth.

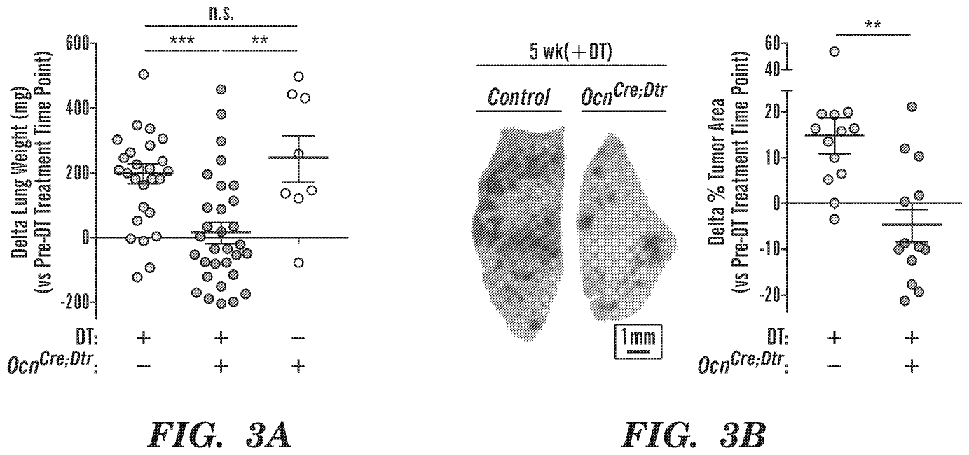

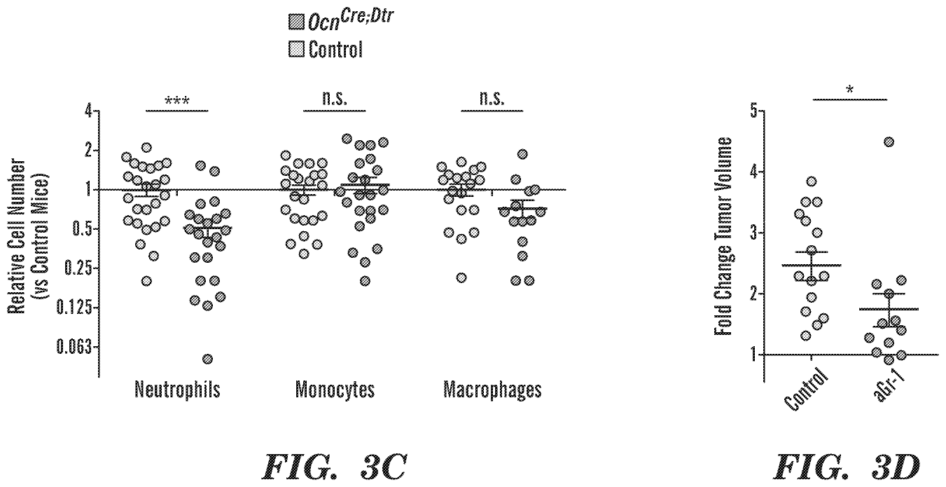

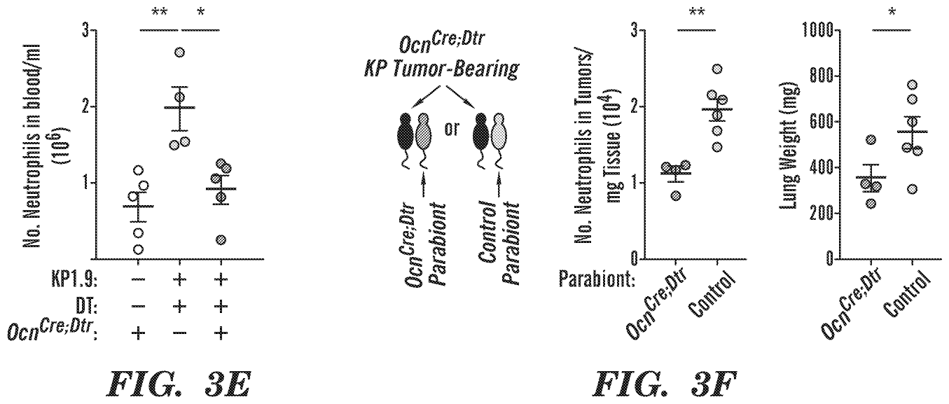

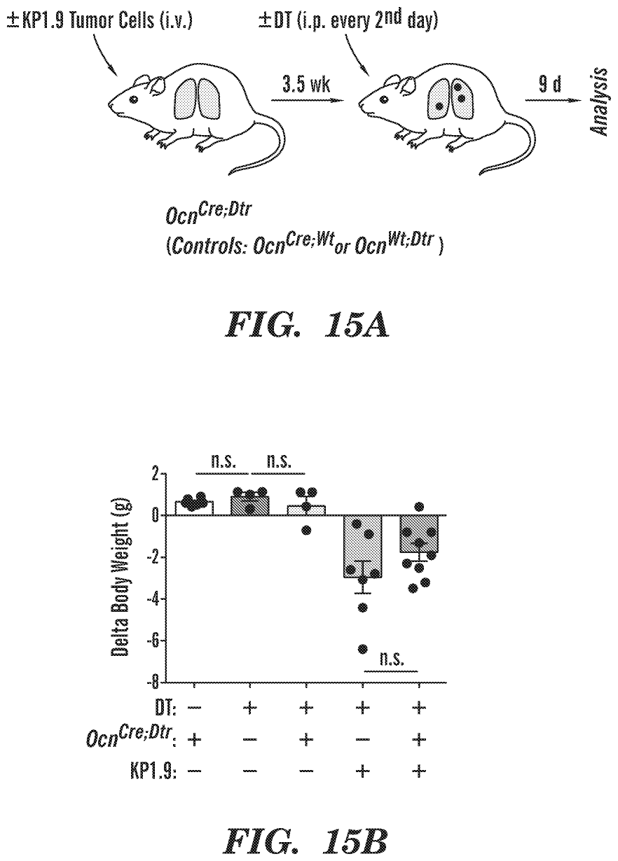

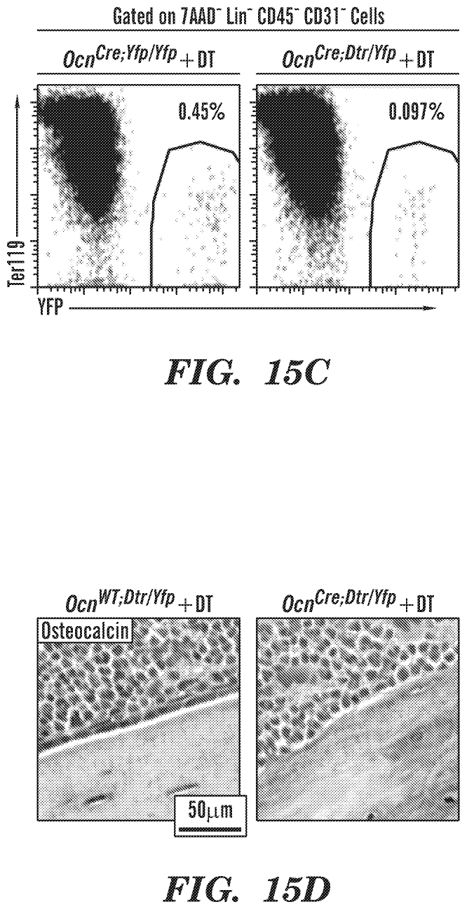

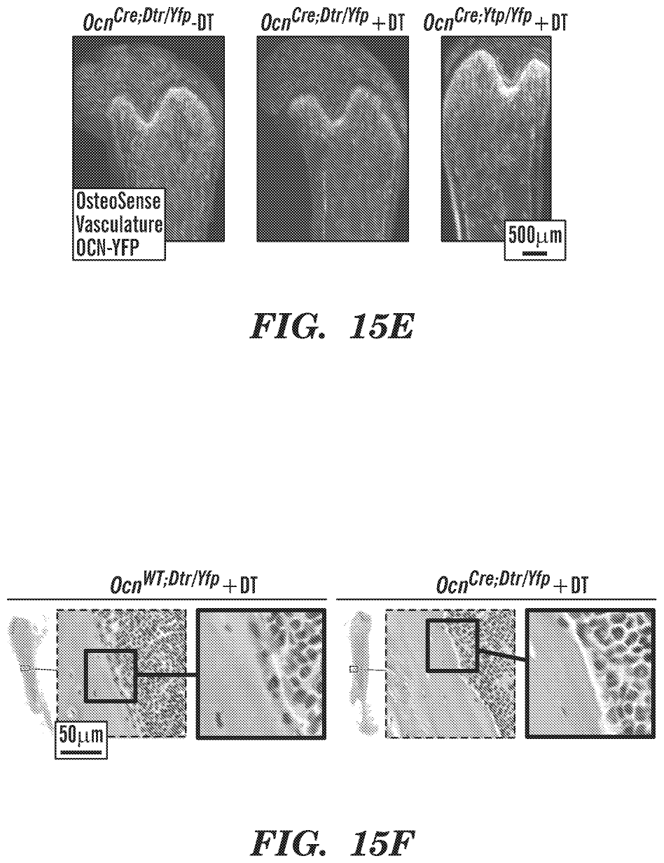

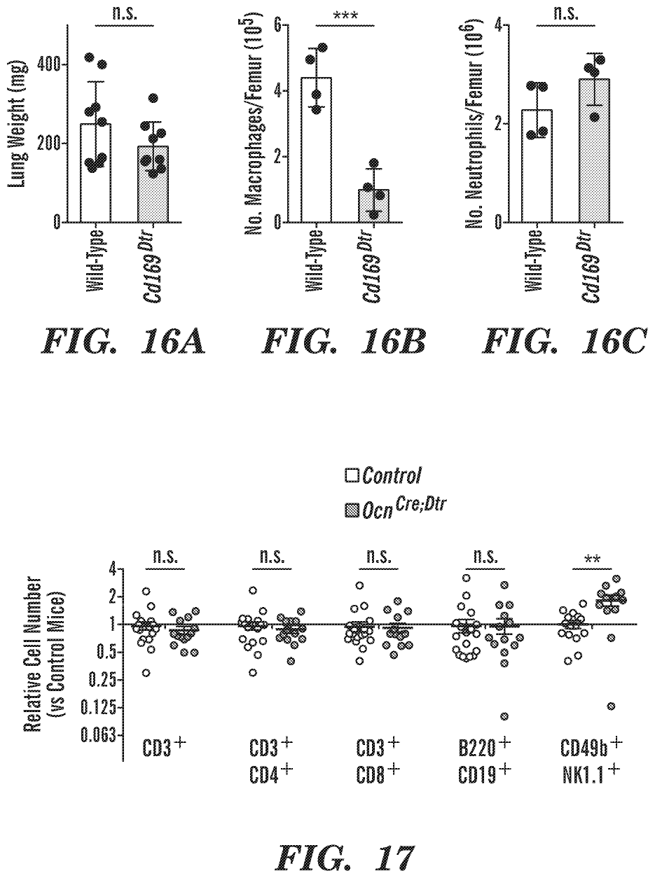

To investigate whether bone marrow osteoblastic cells remotely regulate lung cancer growth, tumor progression in Ocn^{Cre;Dtr} mice were analyzed; Ocn^{Cre;Dtr} mice is a model in which Ocn⁺ cells can be reduced by diphtheria toxin (DT) injection. Ocn^{Cre;Dtr/Yfp} mice were also used to track Ocn⁺ cells based on YFP expression. DT treatment did not affect body weight (FIGS. 15A and 15B) but significantly reduced Ocn⁺ cell numbers, as detected by flow cytometry, immunohistochemistry, in situ microscopy and bone histomorphometry (FIGS. 15C-15F). These analyses confirmed that DT treatment of Ocn^{Cre;Dtr} mice resulted in efficient reduction of osteoblastic lineage cells. Importantly, DT treatment in Ocn^{Cre;Dtr} mice was sufficient to interrupt the progression of established KP lung tumors (FIGS. 3A and B). Control experiments further indicated that tumor reduction required Ocn⁺ cell targeting because DT treatment did not suppress KP tumor progression in mice lacking the Ocn^{Cre} or DT receptor (DTR) transgenes (FIG. 3A). Suppression of tumor growth was not due to nonspecific DT-induced cell death in the bone marrow because DT targeting of CD169⁺ bone marrow cells did not suppress KP lung tumor progression in Cd169^{Dtr} mice (FIG. 16). These findings indicate that Ocn⁺ cells affect lung tumor progression.

Ocn⁺Osteoblasts Supply Tumor-Infiltrating Neutrophils.

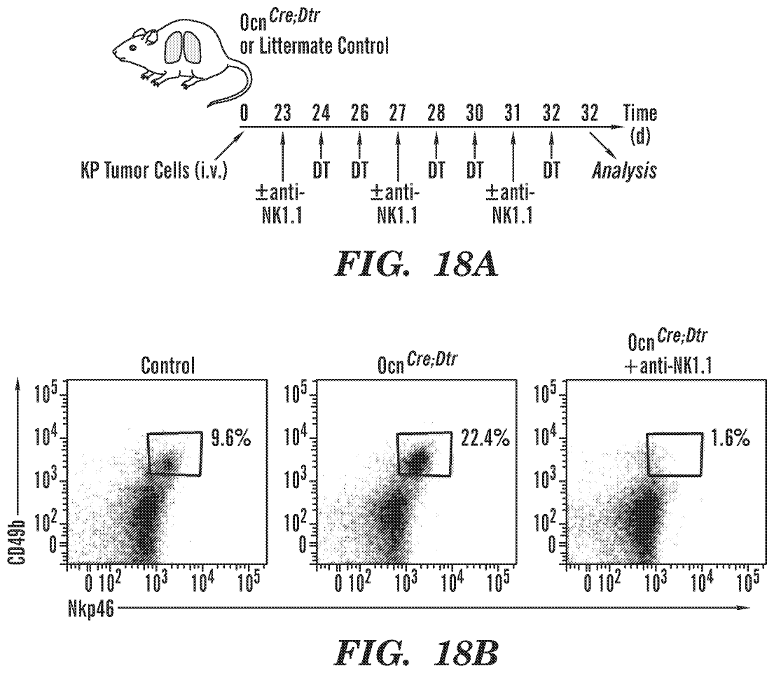

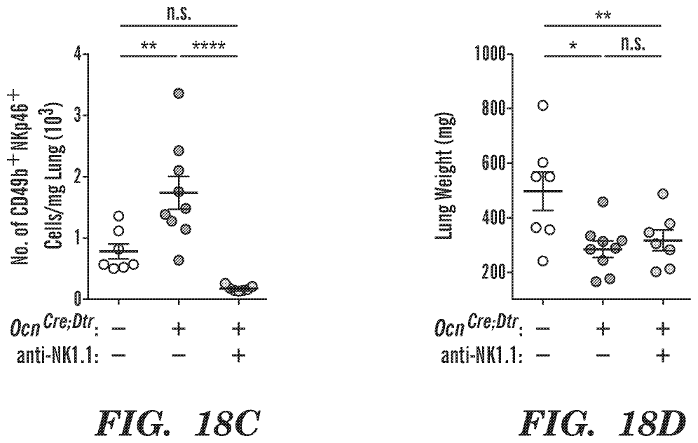

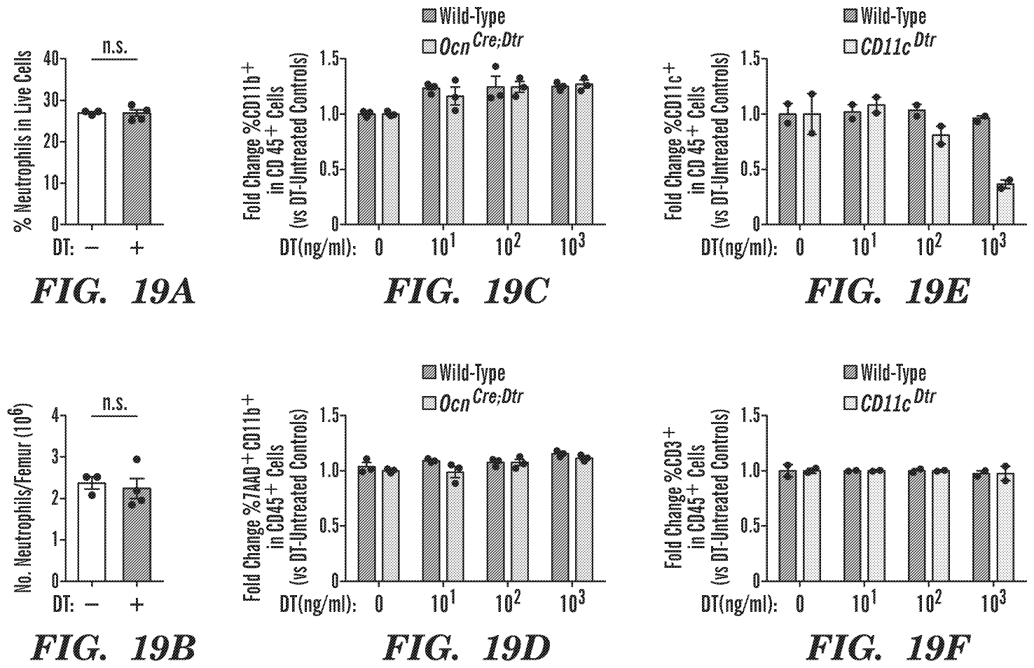

Without wishing to be bound by a particular theory, it was hypothesized that Ocn⁺ cells can affect lung cancer growth by supplying specific hematopoietic cells to the tumor microenvironment. Thus, KP lung tumor immune infiltrates in mice with either unmanipulated or reduced Ocn⁺ cell numbers were compared. Similar pools of monocytes, lung macrophages, B cells and T cells were found in both cohorts; however, mice with fewer Ocn⁺ cells showed a ˜2-3-fold reduction in CD11b⁺ Ly-6G⁺ neutrophils (FIGS. 3C and 17). These mice also had more CD49b⁺ NK1.1⁺ NK cells (FIG. 17), which were likely not required for KP tumor control because NK cell depletion did not restore cancer growth in these mice (FIGS. 18A-18D). Importantly, DT did not target neutrophils directly because wild-type mice treated with DT maintained their neutrophil counts (FIGS. 19A and 19B). Also, CD11b⁺ myeloid cells from Ocn^{Cre;Dtr} mice were not killed by DT in vitro, confirming no functionally relevant DT receptor expression by these cells (FIGS. 19C and 19D), whereas positive control experiments showed DT's ability to kill DTR⁺ cells in vitro (FIGS. 19E and 19F).

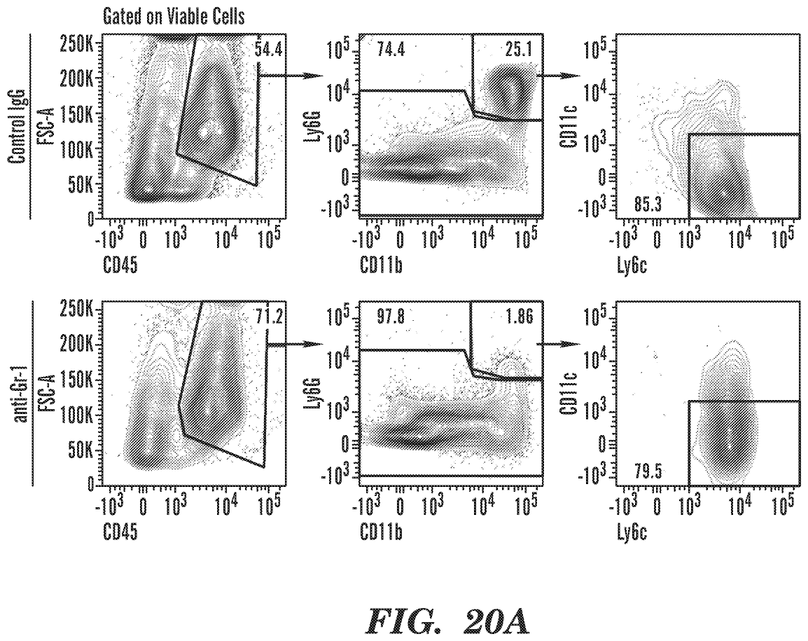

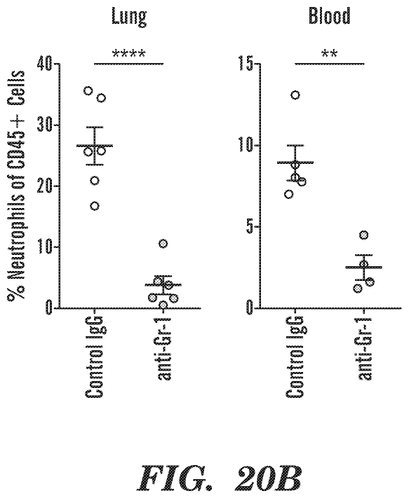

It was next considered whether controlled KP tumor progression in Ocn⁺ cell-reduced mice involves the altered neutrophil response. In this scenario, removing neutrophils would delay growth of the primary tumor even in presence of Ocn⁺ cells. Accordingly, targeting neutrophils with depleting antibodies (FIGS. 3D, 20A and 20B) significantly suppressed KP lung tumor progression in Ocn⁺ cell-sufficient mice, as defined by longitudinal and noninvasive μCT monitoring of lung tumor nodules (FIG. 3D).

To assess Ocn⁺ cells' impact on the tumor-induced systemic neutrophil response, the number of circulating neutrophils in presence or absence of KP lung tumors were compared when Ocn⁺ cells were depleted or not. It was found that the presence of KP tumors was associated with a significant increase in the number of (CD11b⁺Ly6G⁺) neutrophils in the blood (FIG. 3E). This response required Ocn⁺ cells because the number of circulating neutrophils did not increase when Ocn⁺ cells were depleted. These data indicate that Ocn⁺ cells are required for the amplification of tumor-associated circulating neutrophils.

To further test this idea, tumor microenvironments were assessed when the circulatory systems of mice were joined by parabiosis. It was found that joining osteoblast-reduced mice to osteoblast-sufficient mice increased tumor-infiltrating CD11b⁺ Ly-6G⁺ neutrophil numbers in the former by 2.6±0.3 fold (mean±SEM) (FIG. 3F). These neutrophil numbers were comparable to those seen in control (osteoblast-sufficient, nonparabiosed) mice (2.2±0.4 fold; p=n.s.). Importantly, tumors in osteoblast-reduced mice grew faster when joined to osteoblast-sufficient parabionts (FIG. 3E) and similarly to tumors in control (osteoblast-sufficient, nonparabiosed) mice (lung weights: 557.1±68.34 mg and 551.4±30.24, respectively, p=n.s.). Thus, both tumor neutrophil counts and tumor progression were restored in osteoblast-reduced mice when parabiosed to Ocn⁺ cell sufficient mice. Combined, these data not only show that Ocn⁺ cells contribute tumor-infiltrating neutrophils, but also indicates\that these cells display tumor-promoting functions.

Ocn⁺ Cell-Driven Neutrophils Promote Cancer Growth.

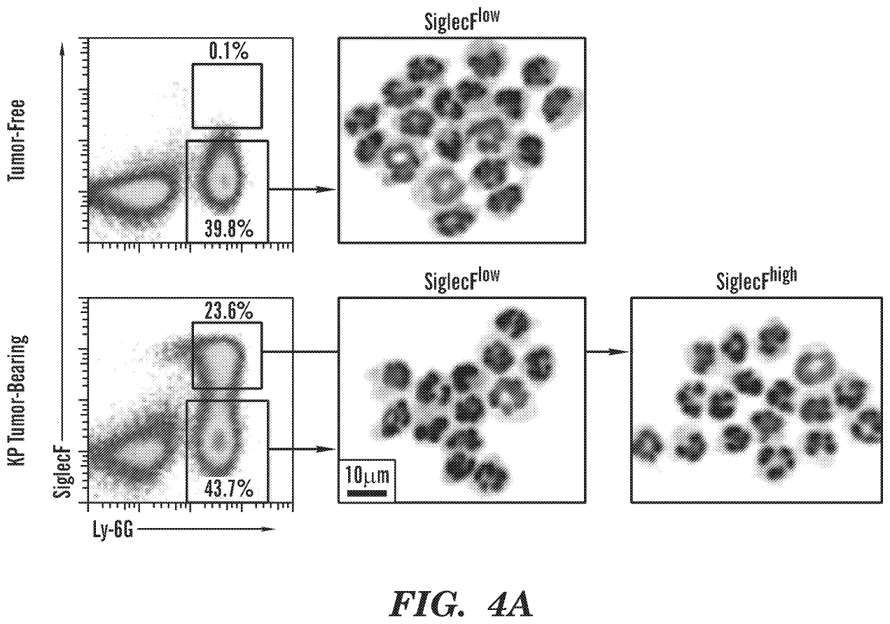

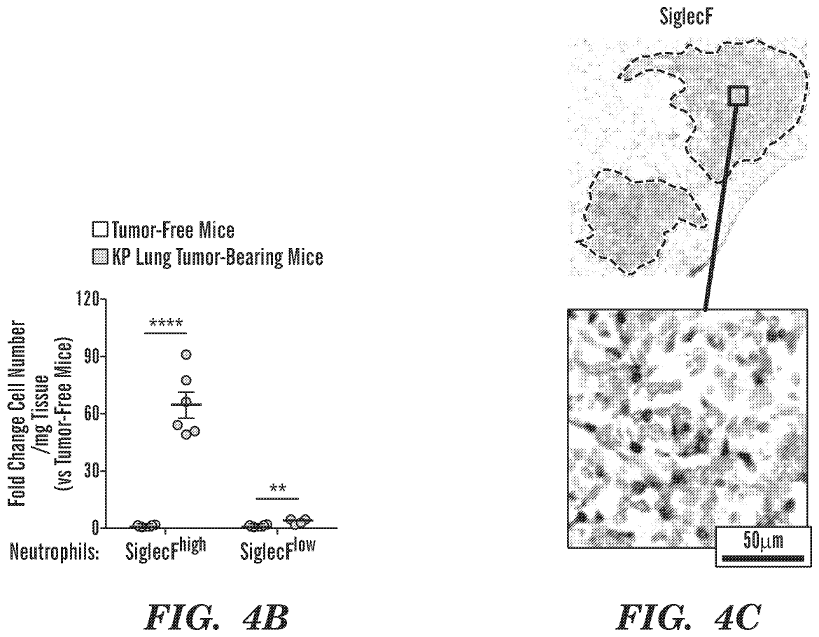

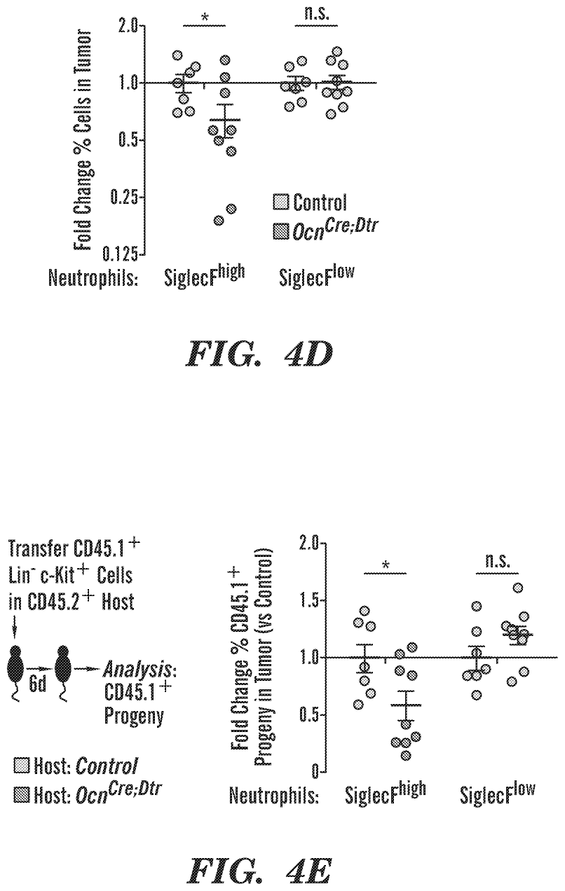

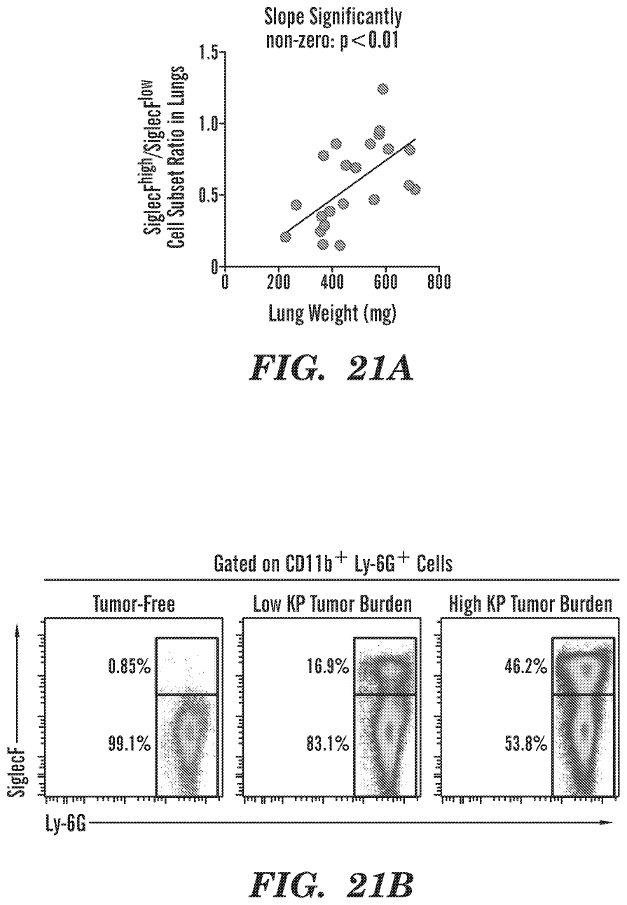

Neutrophils are heterogenous (1) and therefore it was contemplated whether those supplied by Ocn⁺ cells have distinct attributes that can accelerate tumor progression. To address this question, lung neutrophil phenotypes were further assessed. It was found that CD11b⁺ Ly-6G⁺ cells can be divided into two subsets according to expression levels of the lectin SiglecF (FIG. 4A). The SiglecF^{low} subset appeared in high numbers in healthy lungs and expanded only slightly in lungs from tumor-bearing mice; by contrast, the SiglecF^{high} subset was rare in the healthy tissue but expanded ˜70-fold in tumor-bearing lungs (FIGS. 4A and 4B). The SiglecF^{high}/SiglecF^{low} cell subset ratio positively correlated with KP lung tumor burden (FIGS. 21A and 21B), further indicating that the SiglecF^{high} subset continued to accumulate in growing tumors.

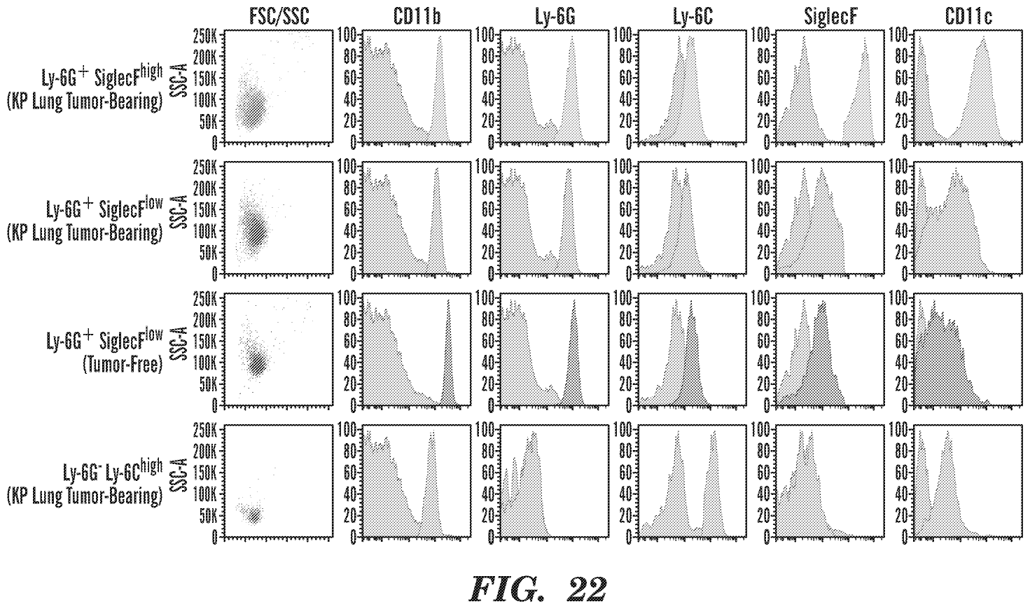

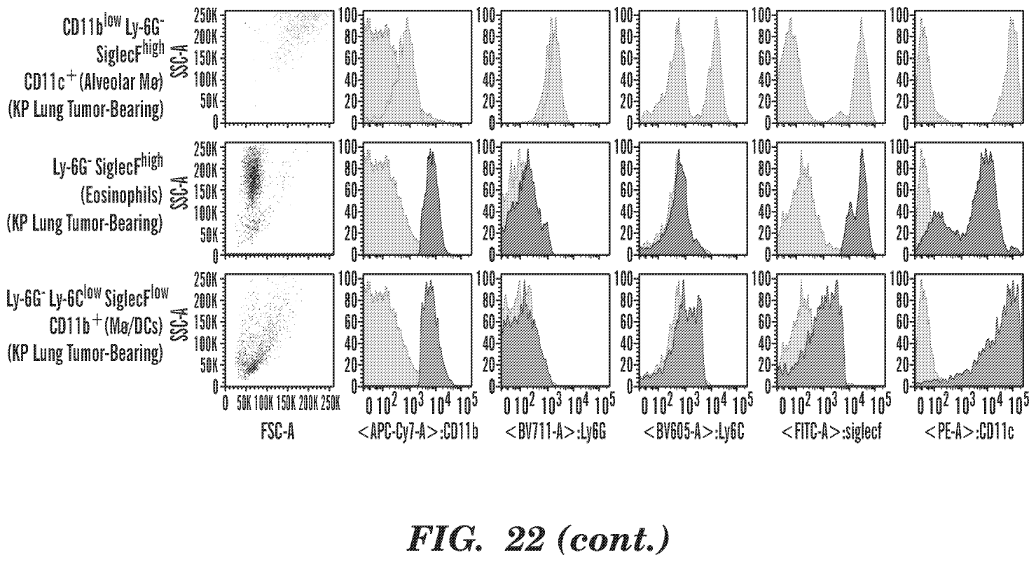

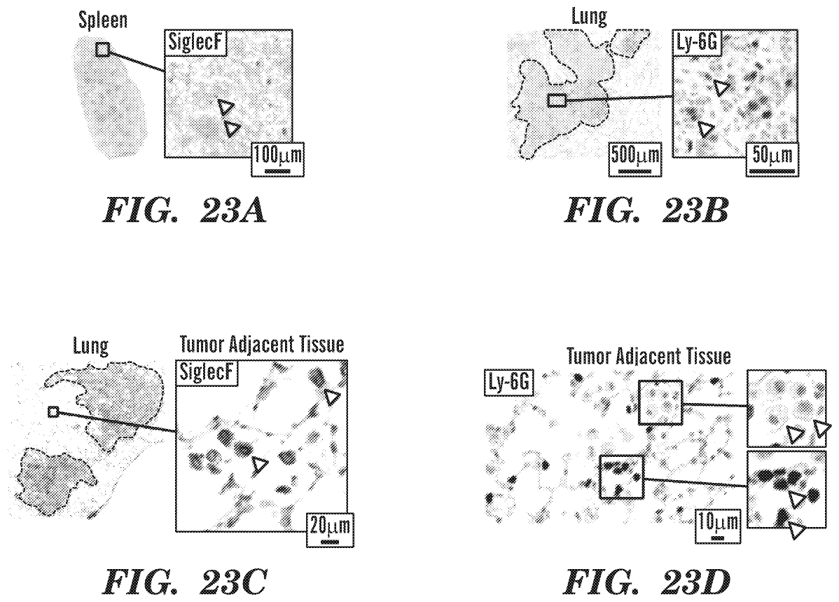

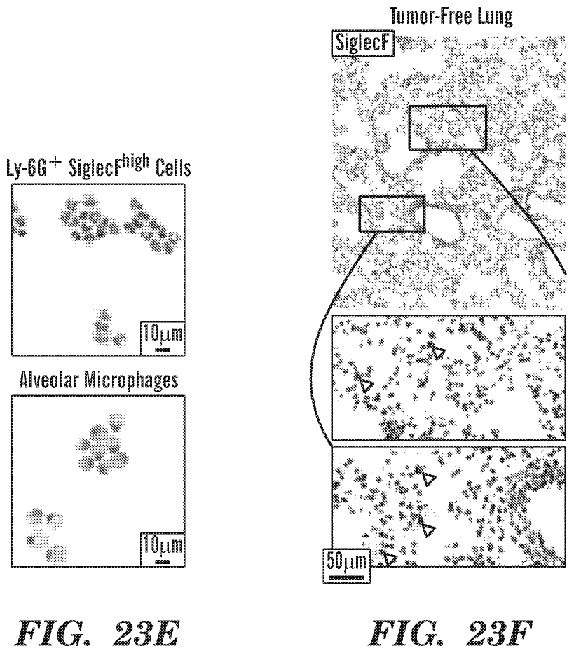

Both the cell surface phenotype and forward/side scatter profiles of the SiglecF^{high} cells closely resembled those of neutrophils and were distinct from those of other myeloid cell types including SiglecF⁺ eosinophils and SiglecF⁺ alveolar macrophages (FIG. 22). Immunohistochemical SiglecF and Ly-6G staining further revealed the presence of Ly-6G⁺ and SiglecF^{high} neutrophil-like cells within tumor nodules (FIGS. 4C, 23A, and 23B), indicating that the SiglecF^{high} neutrophils localize proximal to tumor cells. SiglecF⁺ cells outside the tumor stroma instead mainly resembled alveolar macrophages based on their morphology and Ly-6G⁻ phenotype (FIGS. 23C-23F).

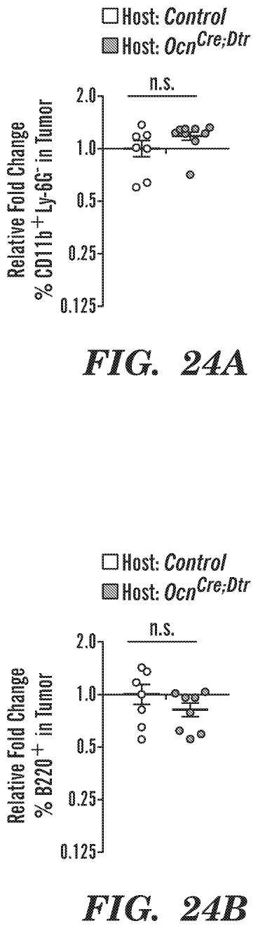

To study whether osteoblasts preferentially contribute SiglecF^{high} lung neutrophils, both SiglecF^{high} and SiglecF^{low} subsets were quantified in tumor-bearing mice with reduced or unchanged Ocn⁺ cell numbers. It was found that Ocn⁺ cell deficiency significantly reduced the percentage of SiglecF^{high}, but not SiglecF^{low}, neutrophils (FIG. 4D). These data suggest that Ocn⁺ cells promote SiglecF^{high} neutrophil accumulation in tumors. To further investigate whether SiglecF^{high} neutrophil accumulation in tumors requires Ocn⁺ cells, the fate of wild-type donor CD45.1⁺ c-Kit⁺ hematopoietic cells were mapped upon adoptive transfer into CD45.2⁺ tumor-bearing recipient mice that had either reduced or unchanged Ocn⁺ cell numbers. It was found that the c-Kit⁺ donor cells' ability to produce SiglecF^{high} lung neutrophils was impaired in Ocn⁺ cell-reduced mice (FIG. 4E). These findings indicate that SiglecF^{high} neutrophil accumulation in tumors depends on Ocn⁺ osteoblastic cells. By contrast, the c-Kit⁺ donor cells were equally able to produce tumor-infiltrating SiglecF^{low} neutrophils (FIG. 4E), as well as macrophages (FIG. 24A) and B cells (albeit at frequencies >25-times lower than myeloid cells; FIG. 24B), in Ocn⁺ cell-reduced and sufficient mice. Donor-derived T cells were very rare or undetectable in the tumor stroma. These findings indicate that KP tumor accumulation of SiglecF^{high} neutrophils, in contrast to other immune cells, depends on Ocn⁺ cells.

SiglecF^{high} Neutrophil Profiling Reveals Cancer-Promoting Phenotypes.

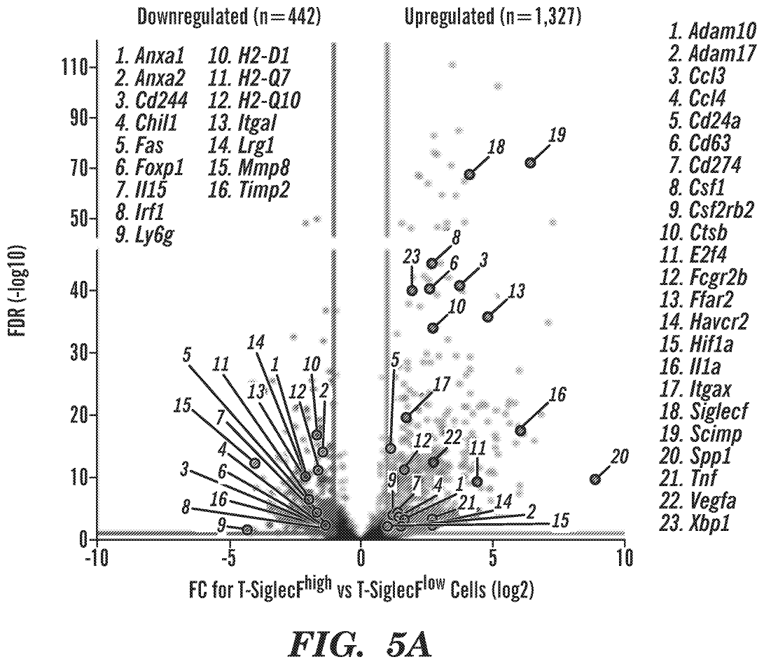

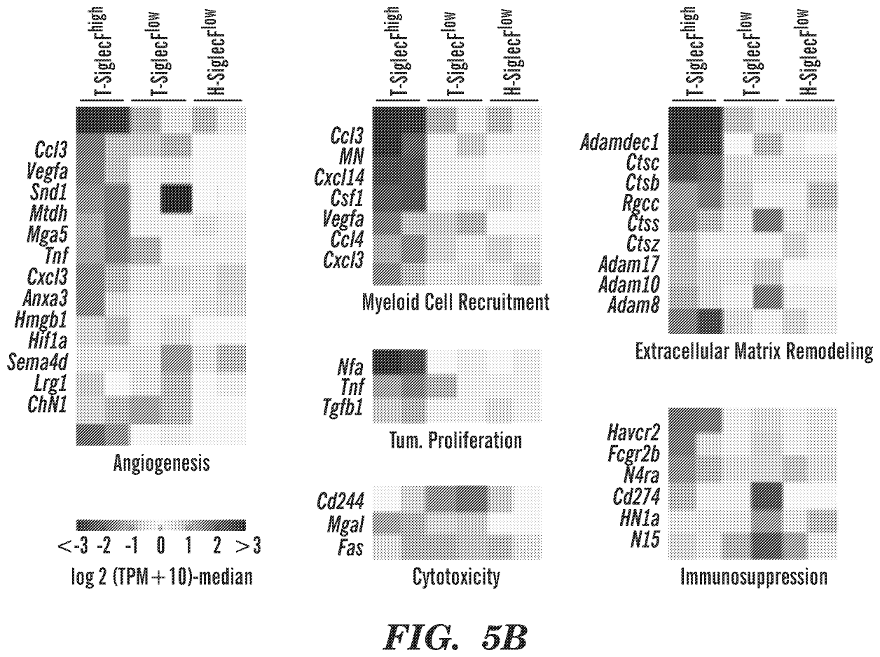

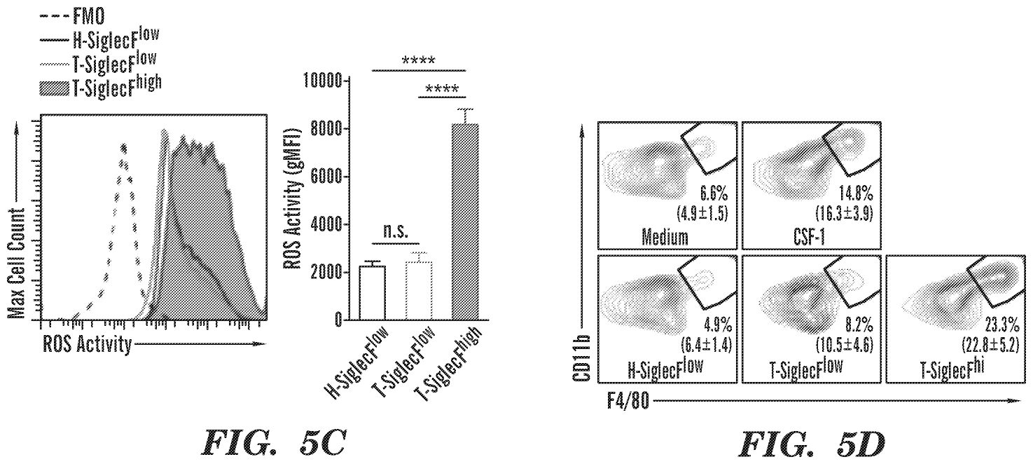

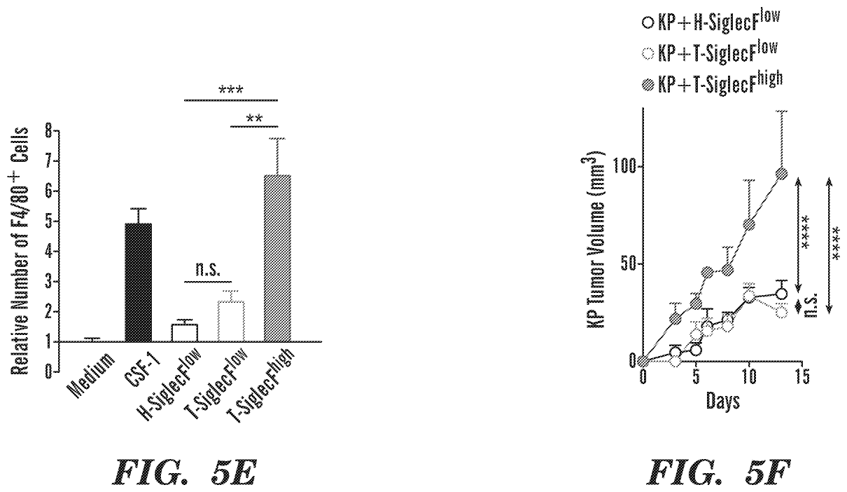

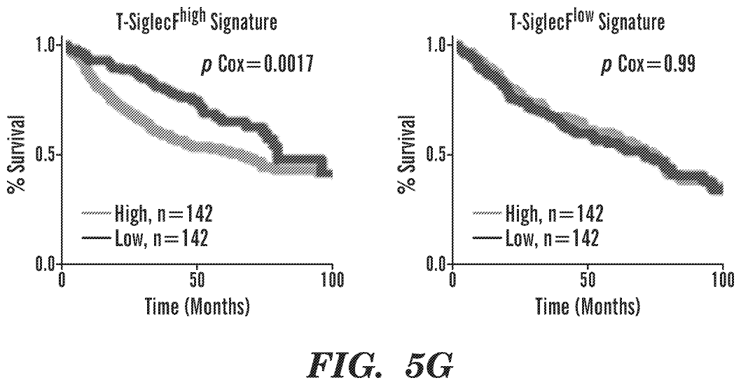

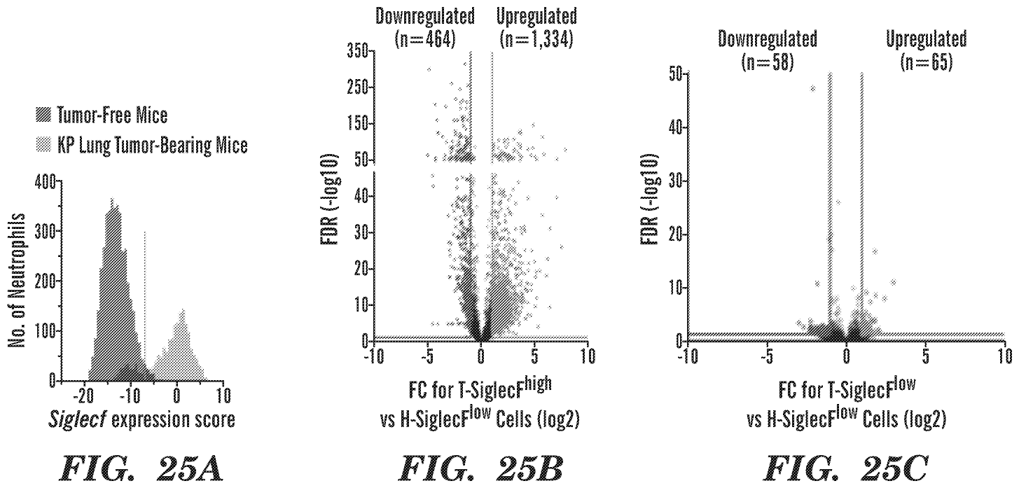

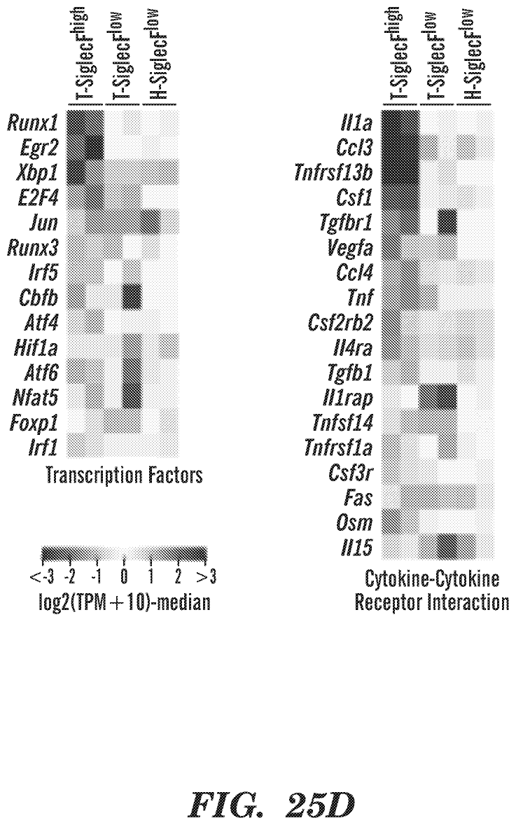

Whether SiglecF^{high} neutrophils in mice have cancer-promoting properties was next evaluated. To this end, the single-cell transcriptomic data of neutrophils from healthy lungs or KP tumors was interregated. By defining a single-cell SiglecF expression score Table 1; detailed in the Methods and Materials section) it was confirmed that neutrophils in healthy lungs were SiglecF^{low}, whereas tumor tissue contained both SiglecF^{low} and SiglecF^{high} subsets (FIG. 25A). Thus, gene expression of three neutrophil populations was compared: (1) SiglecF^{high} cells in tumor-bearing lung (T-SiglecF^{high}; n=1,502 cells), (2) SiglecF^{low} cells in tumor-bearing lung (T-SiglecF^{low}; n=273), and (3) SiglecF^{low} cells in healthy lung (H-SiglecF^{low}; n=4,245). Differential gene expression analysis revealed that T-SiglecF^{high} cells substantially diverged from both T-SiglecF^{low} and H-SiglecF^{low} cells (1,769 and 1,798 differentially expressed genes, respectively; FIGS. 5A and 25B). T-SiglecF^{low} and H-SiglecF^{low} cells were more similar (123 differentially expressed genes; FIG. 25C).

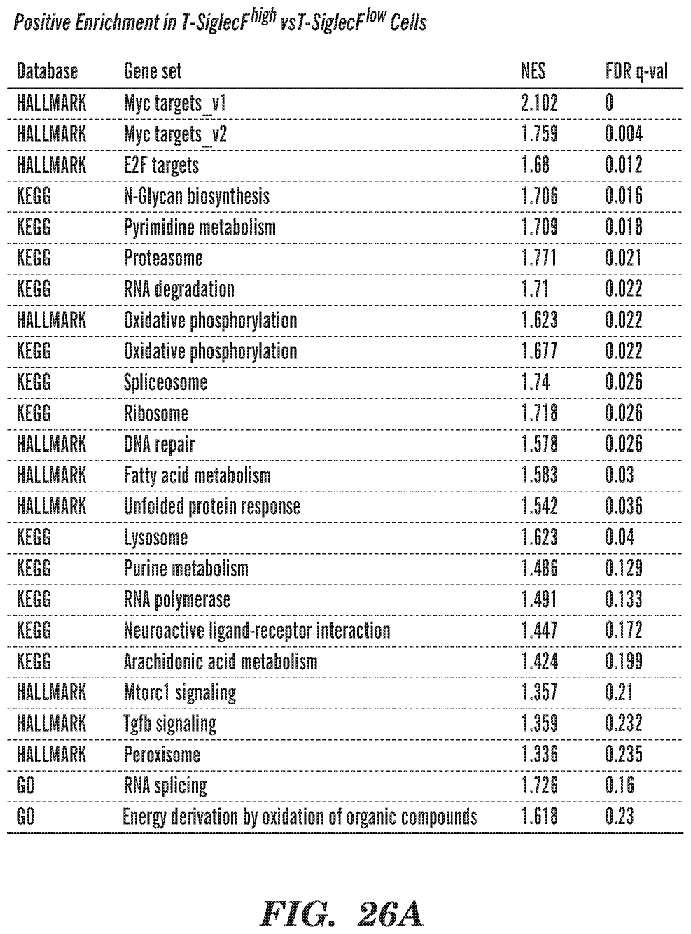

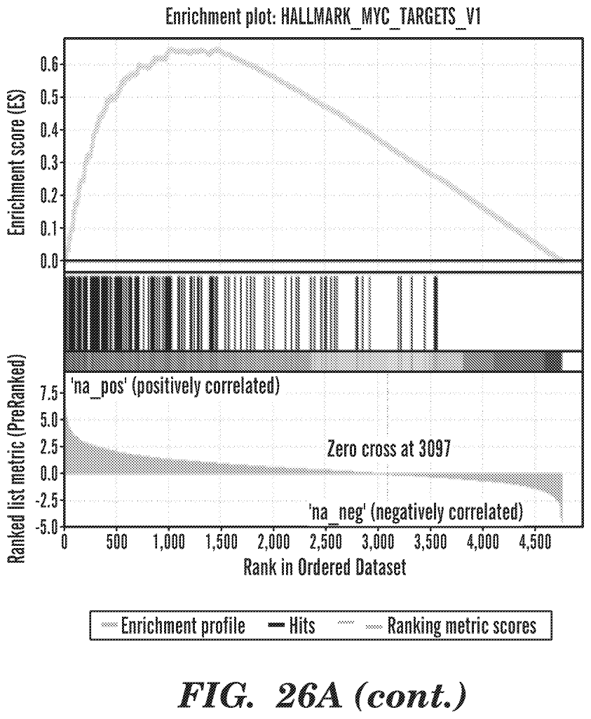

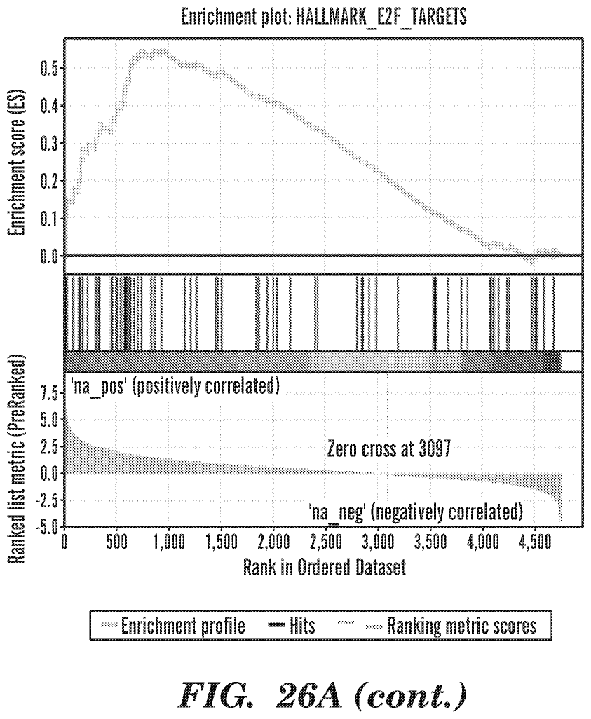

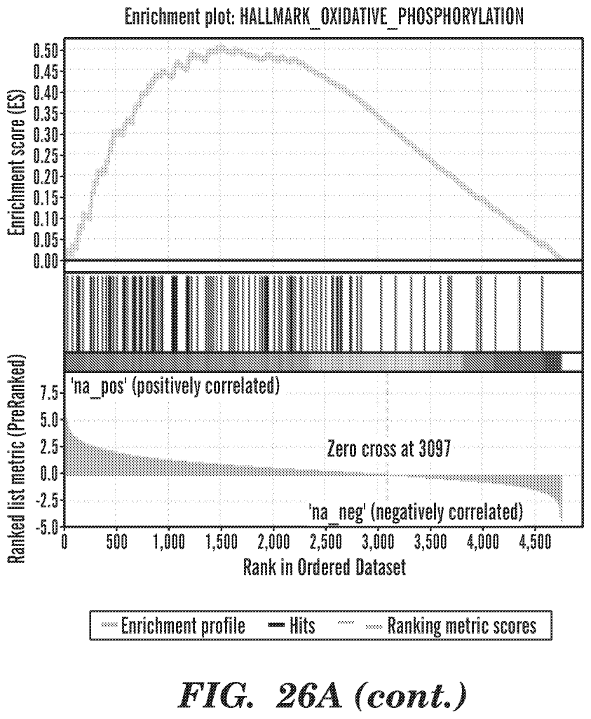

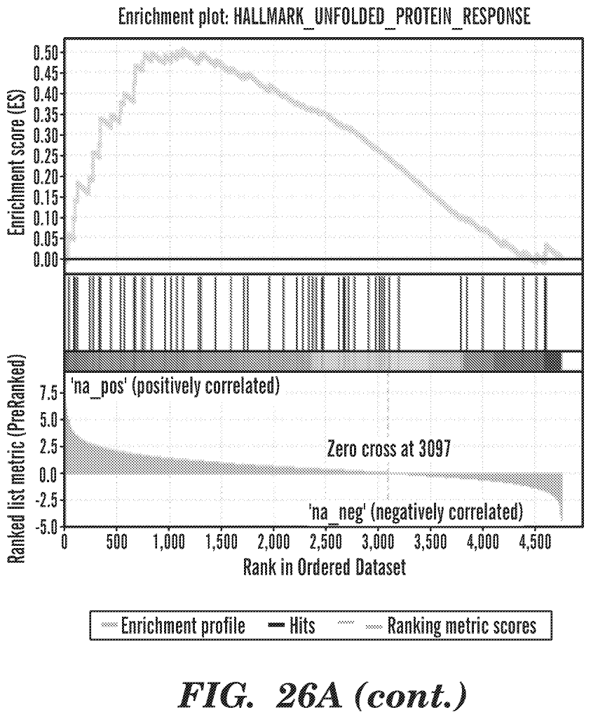

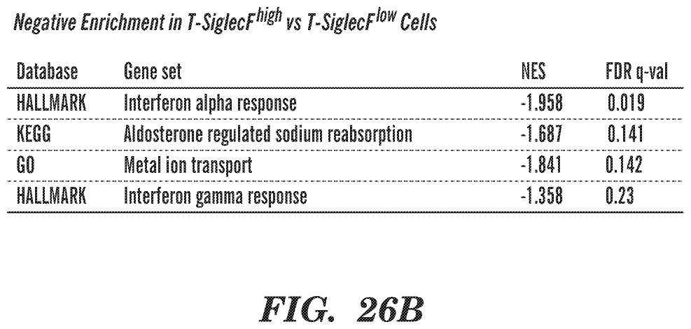

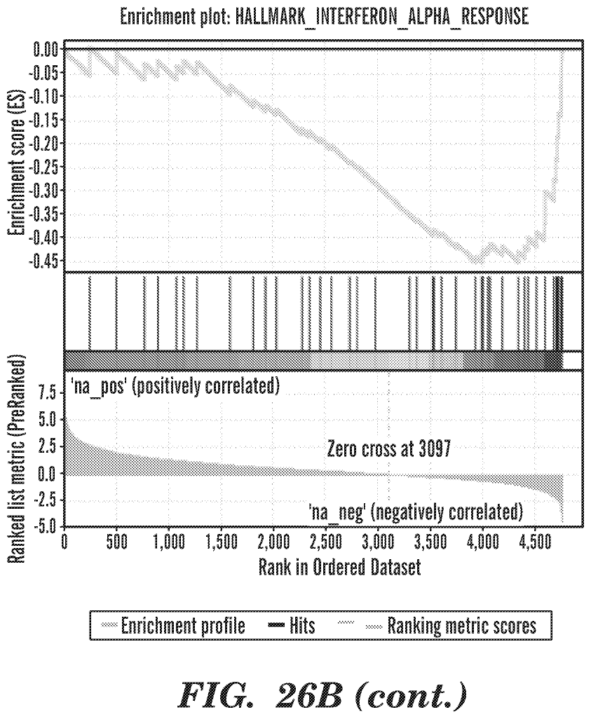

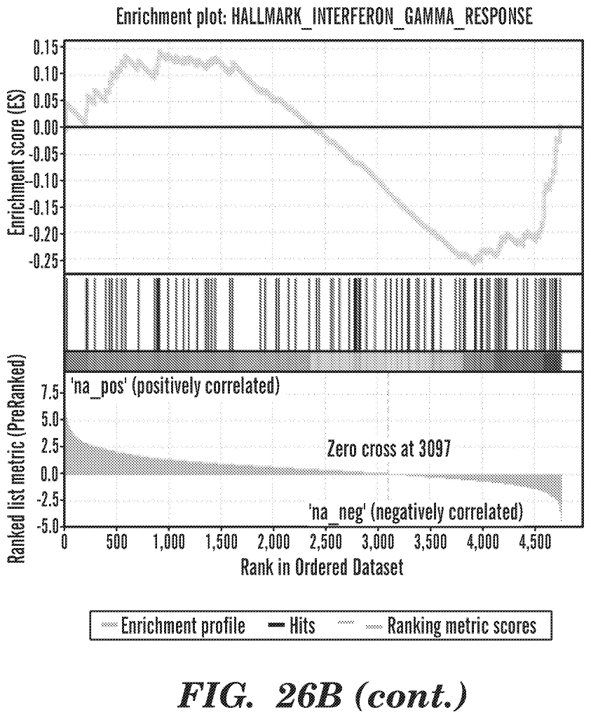

T-SiglecF^{high} cells selectively upregulated the expression of genes associated with tumor-promoting processes (FIGS. 5B and 25D), including angiogenesis (Vegfa, Hif1a, Sema4d), myeloid cell differentiation and recruitment (Csf1, Ccl3, Mif), extracellular matrix remodeling (Adamdec1, Adam17, various cathepsins), suppression of T cell responses (Cd274/PDL1, Fcgr2b, Havcr2) and tumor cell proliferation and growth (Tnf, Tgfb1, Il1a). T-SiglecF^{high} cells also showed decreased expression of genes involved in cytotoxicity (Cd244, Itgal, Fas) (FIG. 5B). Other genes overexpressed in T-SiglecF^{high} cells included, but are not limited to, the ER stress-response gene and transcription factor Xbp1 and the short chain fatty acid receptor Ffar2 (FIG. 5A); Xbp1 impairs myeloid antitumor functions (30) and positively regulates Ffar2 expression (31). Gene set enrichment analysis indicated upregulation of genes involved in oxidative phosphorylation, fatty acid metabolism and glycolysis, suggesting that T-SiglecF^{high} cells undergo a metabolic switch (FIG. 26A). Genes involved in Myc signaling and E2F gene targets were also overexpressed, suggesting that T-SiglecF^{high} cells are more proliferative and resistant to apoptosis (FIG. 26A). Taken together, these findings indicate that SiglecF^{high} neutrophils undergo metabolic changes in the tumor microenvironment and are poised to support tumor-promoting functions, including tumor angiogenesis, tumor cell proliferation, extracellular matrix remodeling and immunosuppressive myeloid cell recruitment.

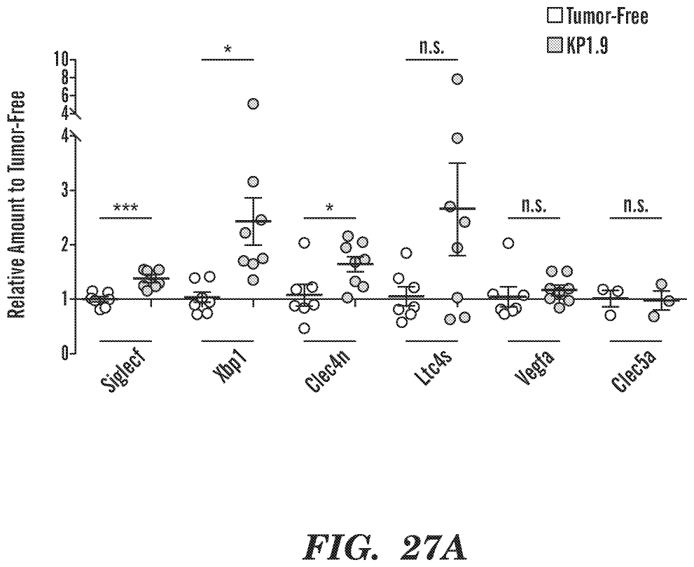

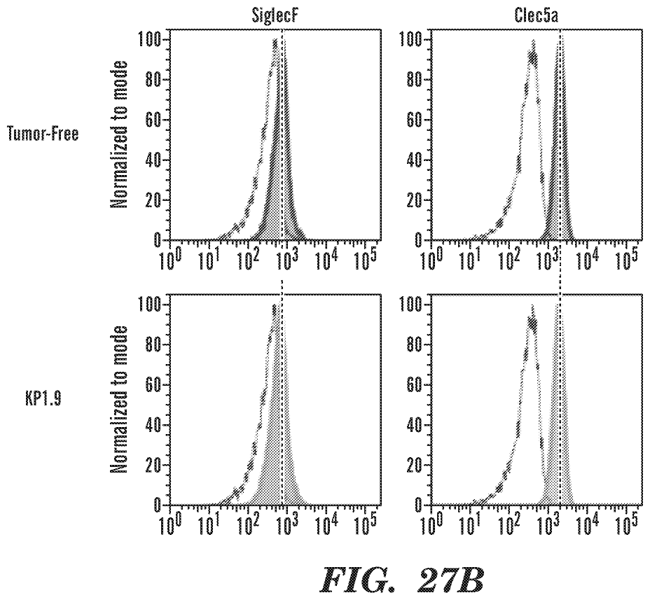

Tumor-infiltrating neutrophils are replenished by circulating cells. Thus, it was explored whether differentiated SiglecF^{high} neutrophils already exist in the blood of tumor-bearing mice. Specifically, CD45⁺ CD11b⁺ Ly6G⁺ neutrophils from the blood of either tumor-free or lung tumor-bearing mice were sorted and assessed for the expression of several genes that were identified to be selectively upregulated by tumor-infiltrating SiglecF^{high} neutrophils, as described above. This analysis revealed increased expression of transcripts corresponding to SiglecF, Xbp1 and Clec4n (and, to a lesser extent, to Ltc4s) in circulating neutrophils from tumor-bearing mice (FIG. 27A). In contrast, the expression of Vegfa and Clec5a was unchanged and flow cytometry analysis showed comparable expression of SiglecF^{high}-associated cell surface proteins (FIG. 27B). These findings indicate that at a minimum, at least some circulating neutrophils acquire molecular features of SiglecF^{high} neutrophils prior to arrival at the tumor site. However, the acquisition of full-fledged SiglecF^{high} neutrophil phenotypes (e.g., as described above) occurs only after the cells have reached their destination tissue. Without wishing to be bound by a particular theory, it is hypothesized that this delay in the onset of a full-fledged SiglecF^{high} neutrophil phenotype limits execution of the cells' effector functions to that site).

SiglecF^{high} Neutrophils Exhibit Cancer-Promoting Functions.

Next, the functions of SiglecF^{high} compared to SiglecF^{low} neutrophils were evaluated. First, the capacity of the different neutrophil populations (T-SiglecF^{high}, T-SiglecF^{low} and H-SiglecF^{low} cells) to produce reactive oxygen species (ROS), which drive diverse pro-tumorigenic inflammatory responses (6, 32, 33), was assessed. To this end, intracellular ROS was measured using an imaging probe that becomes fluorescent upon activation by ROS (6). SiglecF^{high} neutrophils showed increased ROS activity compared to SiglecF^{low} neutrophils in tumor or tumor-free tissue (FIG. 5C), indicating that ROS activity provided by neutrophils is contributed mainly by the SiglecF^{high} subset.

Second, the ability of SiglecF^{high} neutrophils to support other tumor-promoting myeloid cells was assessed. The neutrophil RNAseq analysis (FIG. 5A) revealed that T-SiglecF^{high} neutrophils expressed high levels of the mRNA encoding colony-stimulating factor 1 (CSF-1), which is critical for the differentiation of macrophages from monocytes. In addition, monocyte-derived tumor-associated macrophages drive KP tumor growth (8). Thus, without wishing to be bound by a particular theory, it was hypothesized that SiglecF^{high} neutrophils support cancer progression by promoting the differentiation of tumor-associated macrophages. To test whether T-SiglecF^{high} neutrophils favor macrophage differentiation, monocytes and myeloid precursors were isolated from spleens of tumor-bearing mice and cultured these cells together with either T-SiglecF^{high}, T-SiglecF^{low} or H-SiglecF^{low} neutrophils. Splenic cells cultured with exogenous CSF-1 (instead of neutrophils) served as a positive control. It was found that the presence of T-SiglecF^{high} cells, compared to T-SiglecF^{low} and H-SiglecF^{low}, neutrophils increased the proportion and number (FIGS. 5D and 5E) of F4/80-expressing cells. These findings indicate that SiglecF^{high} neutrophils promote monocyte differentiation into F4/80⁺ macrophages.

Third, whether SiglecF^{high} neutrophils promote cancer growth in vivo was assessed. To this end, tumor-associated SiglecF^{high} neutrophils from tumor-bearing mice were isolated, and, as controls, SiglecF^{low} neutrophils from either tumor-bearing mice or healthy tissue were isolated. Each neutrophil population was mixed with KP tumor cells and the mixture injected intradermally into mice. The relative abilities of these various neutrophil populations to promote KP tumor progression were then assessed. Importantly, it was found that T-SiglecF^{high} cells accelerated tumor growth compared to either T-SiglecF^{low} or H-SiglecF^{low} cells (FIG. 5F). These data suggest that the KP tumor-promoting effects provided by neutrophils are contributed largely by T-SiglecF^{high} cells. Thus, SiglecF^{high} neutrophils exhibit a tumor-promoting transcriptional profile, have increased ROS production, promote macrophage differentiation and boost tumor progression in vivo. Overall the findings described herein indicate that SiglecF^{high} neutrophils have tumor-promoting functions compared to their SiglecF^{low} counterparts.

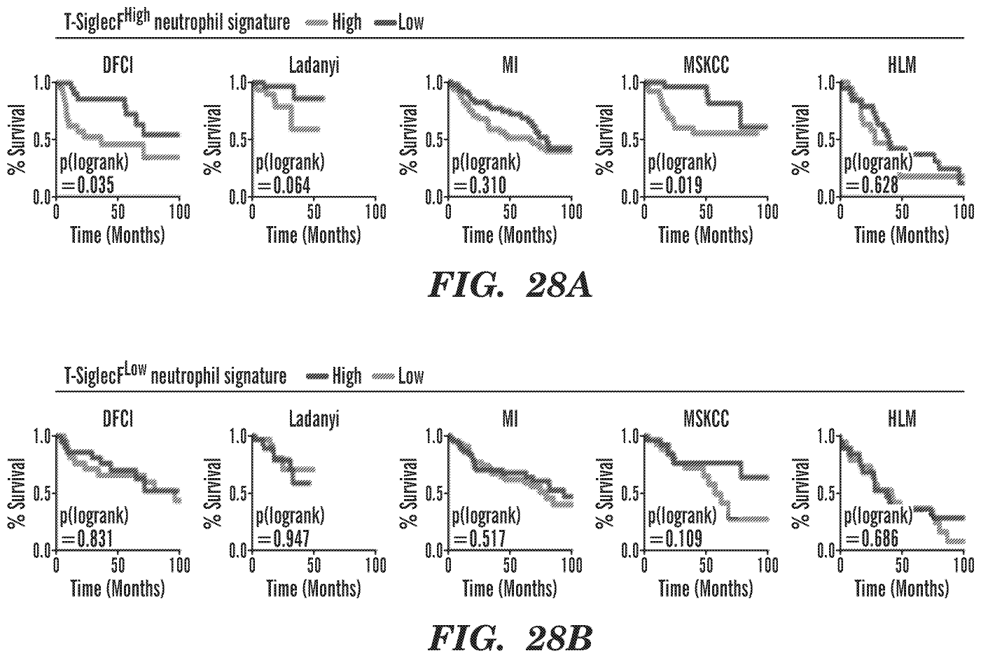

It was next determined whether a mouse SiglecF^{high} neutrophil signature (further detailed in Methods and Materials and in Table 2) might have clinical value. To do this, patient tumor transcriptome and survival data were analyzed (data not shown) (34, 35) and it was determined whether the expression of a SiglecF^{high} neutrophil gene signature was associated with disease outcome in patients with lung adenocarcinoma. A Cox proportional hazards model, controlling for confounding variables, revealed a statistically significant (p=0.0017) association of the SiglecF^{high} neutrophil signature with worse patient survival (FIGS. 5G and 28, and data not shown). In contrast, the SiglecF^{low} neutrophil signature did not associate with disease outcome in lung cancer patients. The survival of the top 25% versus bottom 25% of SiglecF^{high} and SiglecF^{low} neutrophil signature expressers is shown in Kaplan-Meier plots (FIG. 5G).

sRAGE Contributes to the Osteoblast-Induced Neutrophil Response.

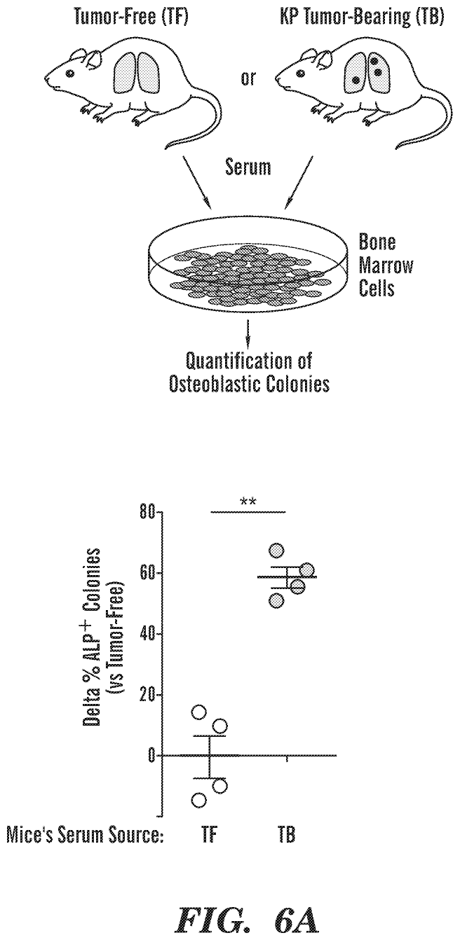

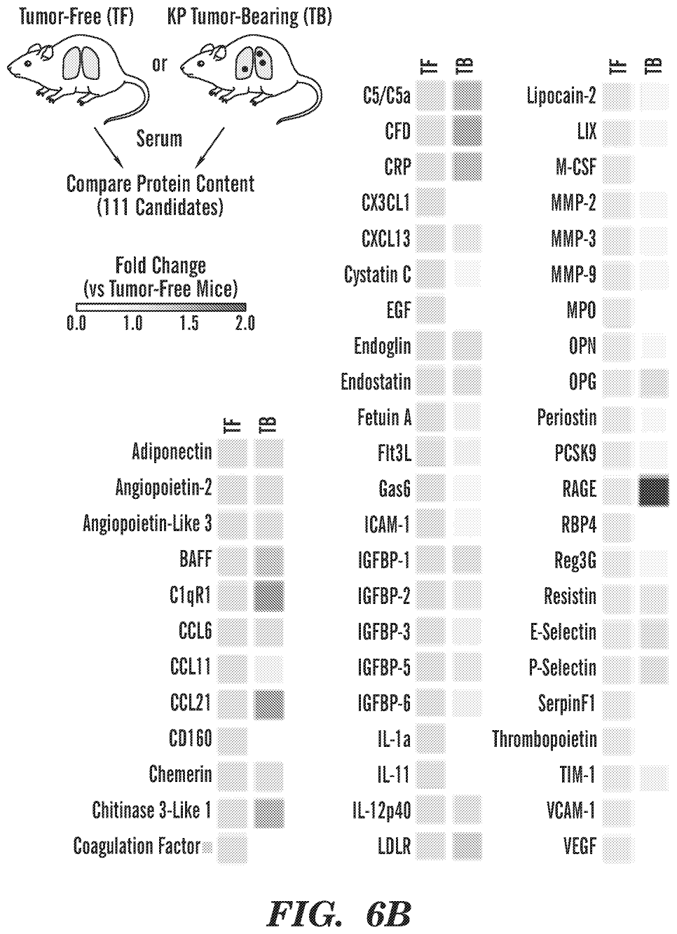

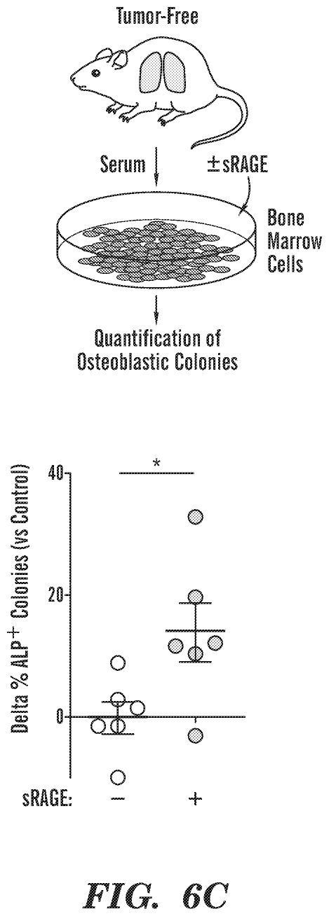

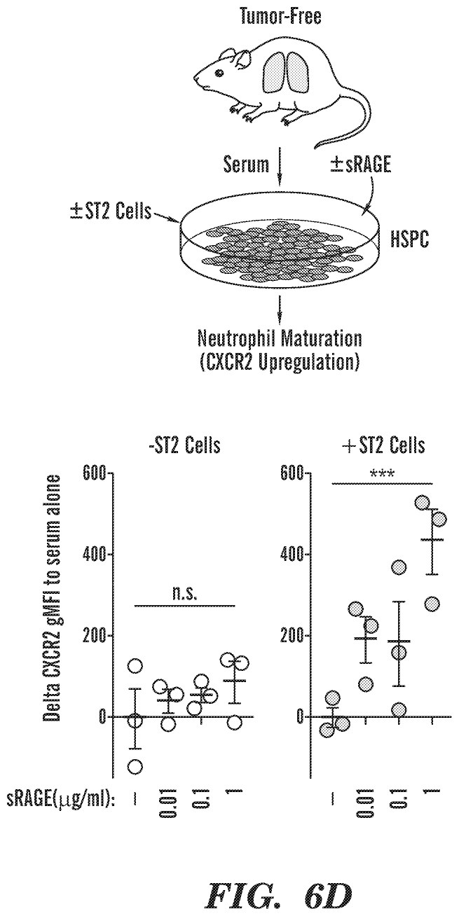

The activation of bone marrow resident cells by distant lung tumors likely involves tumor-induced signals that act over extended distances and stimulate Ocn⁺ osteoblasts. To address the underlying molecular and cellular mechanisms, in vitro experiments were performed to define whether the blood of lung-tumor bearing mice contains factors that increase osteoblastic lineage cell activity. Serum from either lung tumor-bearing or tumor-free mice was collected and added to bone marrow cells cultured under osteogenic conditions. The number of osteoblastic colonies were then quantified by measuring alkaline phosphatase enzymatic staining, as a proxy for osteoblastic activity. As shown in FIG. 6A, serum from tumor-bearing animals significantly increased the number of osteoblastic colonies compared to serum from tumor-free mice. Thus, blood components from tumor-bearing mice are sufficient to promote osteoblastic lineage cells.

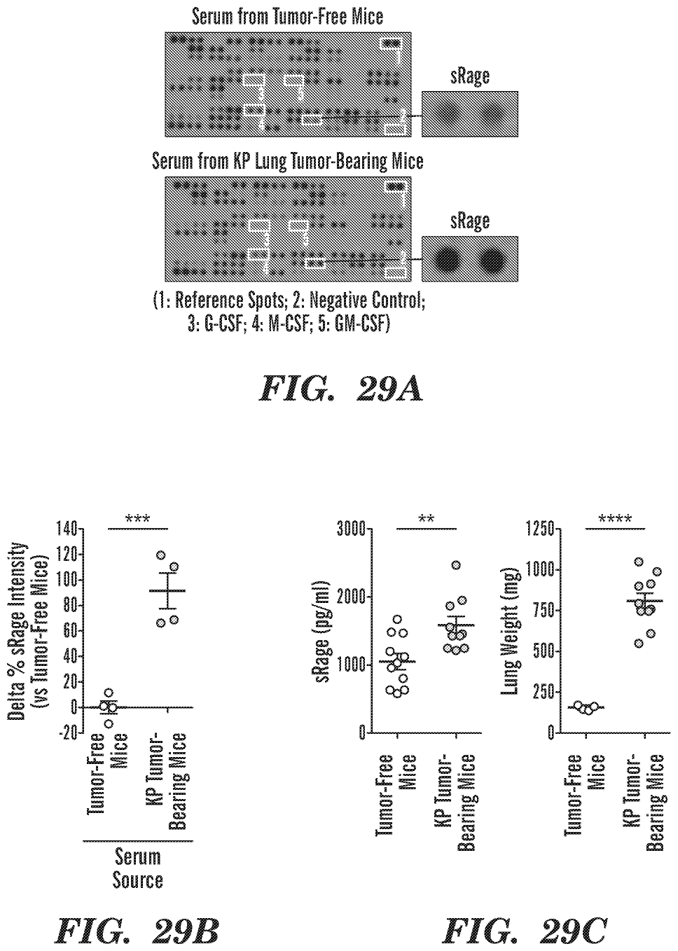

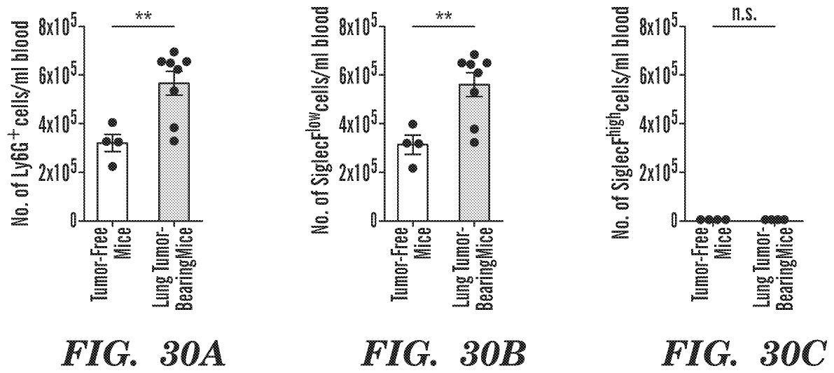

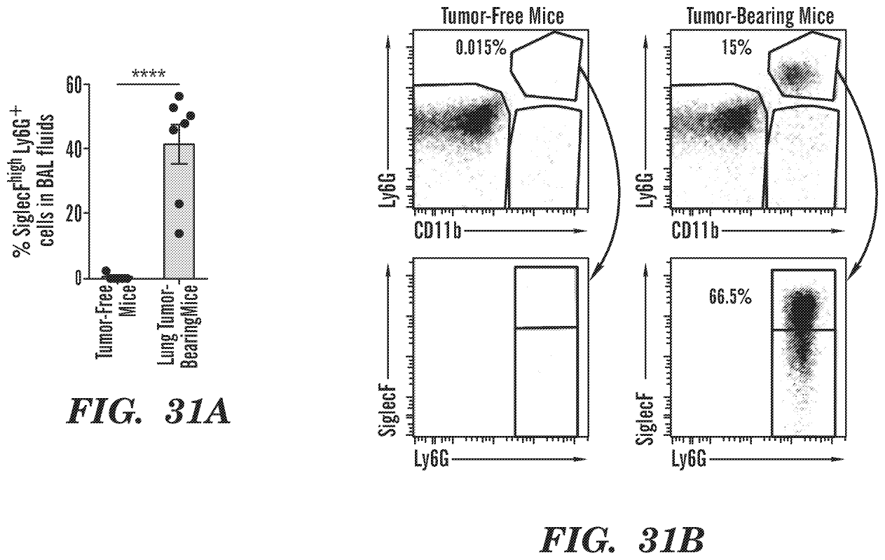

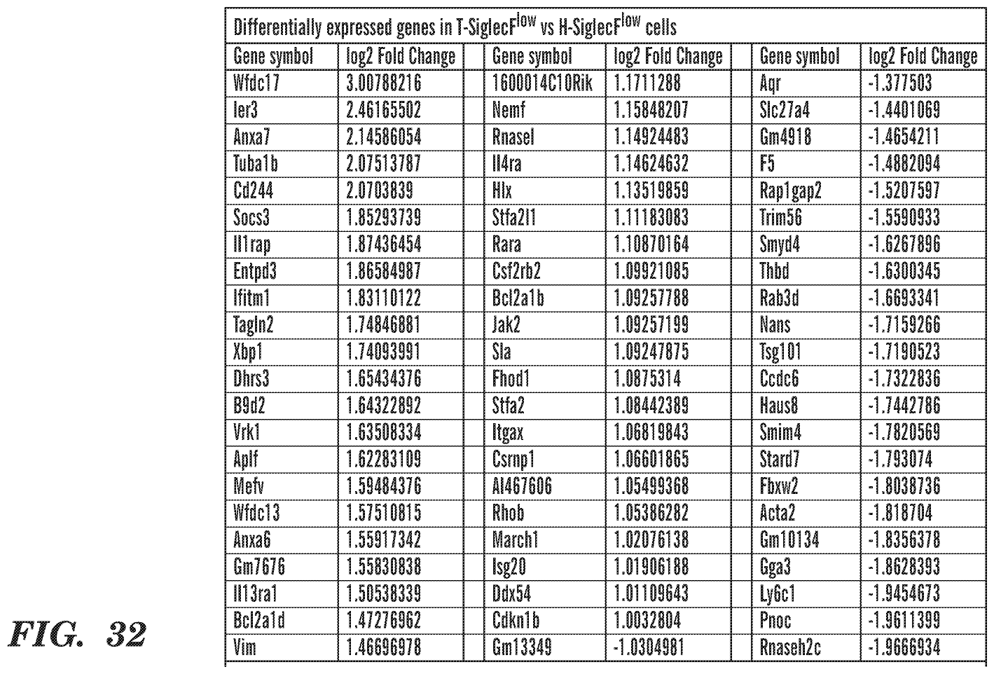

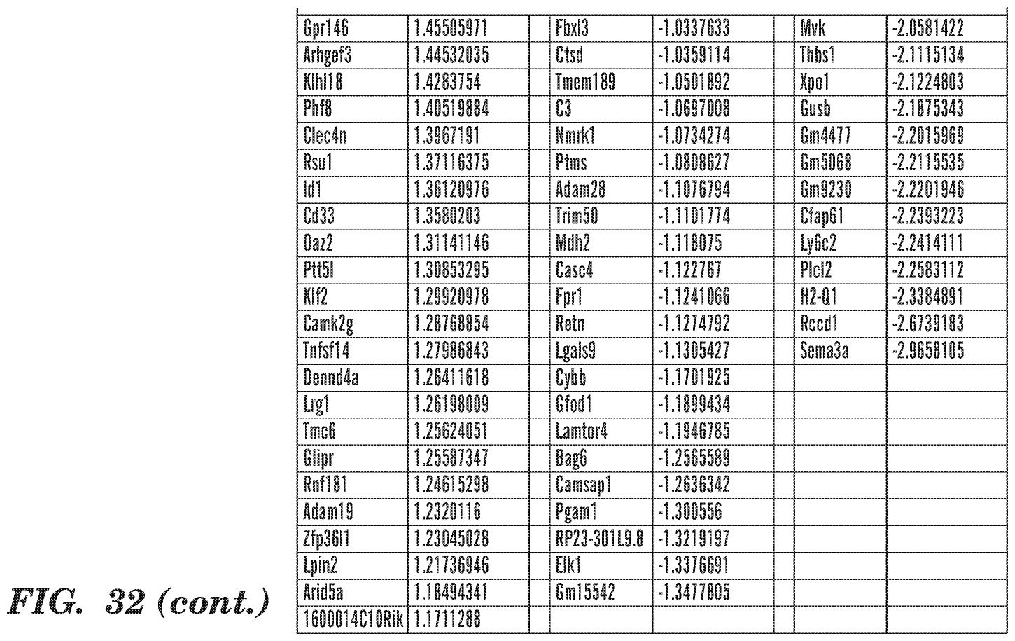

To identify specific serum factors that contribute to increased osteoblast activity, a protein array to quantify 111 cytokines and growth factors in the blood collected from tumor-bearing or tumor-free mice was used. It was found that the concentration of the majority of factors tested was similar in tumor-bearing and tumor-free mice (including, but not limited to, the myeloid growth factors G-CSF, GM-CSF, and M-CSF) whereas some factors (e.g. C1qR1, CCL21, Complement factor D) were slightly enriched in tumor-bearing mice. Notably, the receptor for advanced glycation endproducts (RAGE) was upregulated ˜two-fold in the blood of tumor-bearing mice when compared to tumor-free mice (FIGS. 6B, 29A and 29B). This finding were confirmed using an independent ELISA assay (FIG. 29C).

Various ligands can activate membrane-bound RAGE to trigger pro-inflammatory cascades, a process which has been implicated in several diseases, including diabetes, Alzheimers and cancer. The circulating form of RAGE, referred to as soluble RAGE (sRAGE), can prevent the binding of ligands, including advanced glycation end products, to the RAGE receptor (36). Interestingly, previous studies have connected sRAGE and RAGE ligands to bone regulation (37-39). To test the possibility that sRAGE contributes at least in part to increasing osteoblastic activity, the in vitro osteoblast culture experiment described herein above were repeated, however, it was specifically determined whether supplementing serum from tumor-free mice with sRAGE was sufficient to stimulate osteoblast activation. Indeed, this experimental condition significantly increased osteoblastic colony forming units when compared to control conditions (FIG. 6C).

Finally, it was investigated whether sRAGE could stimulate bone marrow neutrophil maturation and whether this process involved stromal osteoblastic cells. Because developing bone marrow neutrophils require upregulation of CXCR2 expression for release into blood (1), neutrophil expression of this chemokine receptor was tested. To this end, Lin⁻ cKit⁺ bone marrow hematopoietic cells were cultured with or without the ST2 pre-osteoblastic cell line and in the presence of increasing doses of exogenous sRAGE (FIG. 6D). This experiment indicated that the presence of sRAGE increases CXCR2 expression on developing neutrophils. Furthermore, it was found that CXCR2 expression increased only in the presence of bone marrow stromal cells. These preliminary data indicate that tumor-associated factors can stimulate osteoblastic cells, which in turn regulate immune responses.

### Discussion

This study identifies systemic crosstalk between lung tumors and bones: lung adenocarcinomas can remotely activate Ocn⁺ osteoblasts in bones even in the absence of local metastasis. In turn, these osteoblasts supply tumors with SiglecF^{high} neutrophils, which foster cancer progression. The tumor-promoting functions of SiglecF^{high} neutrophils align with previous experimental data showing that neutrophils can promote cancer in various animal models (6, 40-45). The findings also align with human studies, which indicate that a high neutrophil-to-lymphocyte ratio in blood is associated with adverse overall survival in many solid cancers, including that of the lung (10), and that lung adenocarcinoma infiltration by neutrophils is strongly linked to poorer clinical outcome (46). Interestingly, both single-cell transcriptomics and functional studies suggest that SiglecF^{high} neutrophils, but not their SiglecF^{low} counterparts, promote primary tumor growth. These data support the model that tumor-infiltrating neutrophil populations encompass cell subsets with heterogeneous functions (1) and indicate new ways to interrogate the neutrophil response to cancer. Interestingly, the phenotype exhibited by SiglecF^{high} neutrophils resembles at least in part that of granulocytic myeloid-derived suppressor cells (MDSCs) (47). For example, both granulocytic MDSCs and SiglecF^{high} neutrophils express the CD11b and Ly-6G surface markers, upregulate pro-angiogenic factors (e.g. Vegf) and produce high levels of ROS (47).

Osteoblastic-lineage cells are mostly known for their role in the control of bone formation (22, 48, 49), but increasing evidence indicates that these cells can also regulate hematopoiesis (17, 50) with reported impacts on both B cell (51-54) and T cell (52, 55) production at steady-state. Also, genetic perturbations of osteoblast-lineage cells deregulate myelopoiesis and can instigate myeloid hematopoietic malignancies (56-58). Here it is further reported that osteoblastic cells can control tumor-infiltrating SiglecF^{high} neutrophils, i.e., a discrete immune cell subset of the tumor microenvironment. No evidence that osteoblastic cells control tumor-infiltrating B or T cells was found, although it is possible that osteoblast-mediated regulation of lymphocytes or other immune cells occurs in other cancer types. Furthermore, the bone marrow is composed of many different resident cell populations including adipocytes, endothelial cells, hematopoietic cells and nerves, which together with osteoblasts form a complex network that is critical to the production, maturation and egress of hematopoietic populations (13). It is conceivable that some cancers affect multiple bone-resident cell populations, which in turn regulate distinct tumor-associated immune events. The study of additional bone marrow resident cells, for example with Cre-based models (13), can help to capture more fully the complexities of systemic tumor-associated host responses. Additionally, better understanding how lung tumors activate osteoblasts will require further study. Beside the effects of sRAGE identified in this study, it is possible that tumor-bone interactions involve additional components.

In accordance with amplified osteoblastic activity in tumor-bearing mice, increased bone density in lung adenocarcinoma patients was observed. This contrasts with the decrease in bone density that is known to occur in patients with other cancer types (for example, those with Parathyroid hormone-related protein (PTHrP) secreting tumors (59)), or more broadly in cancer patients following certain anticancer therapies (60, 61). In this study the focus is on patients prior to cancer therapy, and individuals with chronic conditions (e.g., rheumatoid arthritis), medication use (e.g., glucocorticoids, bisphosphonates and prior cancer treatment), paraneoplastic syndromes, and osseous metastases were excluded, as these variables can all influence bone density. Because cancer cachexia or smoking can also lead to bone loss, lung cancer patients described herein in the Examples were precisely matched to control individuals with similar body mass index, age and smoking history. Furthermore, because most lung cancer patients undergo chest CT scans with the administration of intravenous contrast, which can artificially increase bone density measurements (62), the analysis was limited to non-contrast CT scans. Based on the analysis of 140 individuals, the findings described herein indicate that in NSCLC patients, the primary tumor alters bone metabolism, resulting in increased bone density. It is contemplated that this mechanism can be utilized by other cancers to promote cancer progression. It will be important to investigate bone parameters in more patients and in various clinical conditions, since the systemic manifestation of cancer is complex and may vary depending on disease stage, tumor type and the tumor's secretory profile.

This study underscores the importance of studying cancer as a systemic disease. Interrogating tumor-associated host responses through this lens should be important to fully address fundamental mechanisms of tumor immunity and effects of cancer therapies. Specifically, considering immune cells as critical therapeutic targets, it will be relevant to broadly investigate hematopoietic organs distant from the primary tumor to uncover ways in which cellular and molecular components at those sites control hematopoietic cell production, maturation and activation in cancer, and how these parameters can be manipulated. Given their involvement in shaping tumor progression, the study described herein posits bone marrow-resident Ocn⁺ cells and SiglecF^{high} neutrophils as relevant clinical biomarkers and candidate vantage points for anticancer therapy.

### Materials and Methods

Mice

Kras^{LSL-G12D/WT}:p53^{Flox/Flox} (referred to as KP) mice were used as a conditional mouse model of NSCLC (20) and bred in the laboratory in the C57BL/6. To track and deplete osteoblastic lineage cells by genetic means, mice that expressed Cre-driven yellow fluorescent protein (YFP) under the control of the osteoblastic cell reporter osteocalcin (Ocn) (49, 63, 64) were generated. In detail, Ocn^{Cre} (B6NFVB-Tg(BGLAP-cre)1Clem/J) transgenic mice were bred to RosaD^{Dtr} (C57BL/6-Gt(ROSA)26Sor^{tm1(HBEGF)Awai}/J) and Rosa^{YfP} mice (B6.129X1-Gt(ROSA)26Sor^{tm1(EYFP)Cos}/J) (Jackson Laboratory) to generate Ocn^{Cre;Dtr}, Ocn^{Cre;Dtr/Yfp} or Ocn^{Cre;Yfp} mice, respectively. Moreover, KPOcn-^{Gfp} mice by breeding KP mice with Ocn^{Gfp-topaz} (C57BL6/Tg(BGLAP-Topaz)Rowe/J) mice (25) were generated. Cd11c^{Dtr} mice (B6.FVB-Tg(Itgax-DTR/EGFP)57Lan/J) were obtained from Jackson Laboratory. Cd169^{Dtr} transgenic mice (Siglec1^{tm1(HBEGF)Mtka}) were provided by the Riken Institute (Japan). Wild type and CD45.1 C57BL/6 mice were purchased from Jackson Laboratory. All animal experiments were performed according to approved IACUC guidelines, except experiments in KP mice for anti-Gr-1 antibody (Ab) depletion that were approved by the Veterinary Authority of the Canton de Vaud, Switzerland (license number VD2391) and the Réseau des animaleries lémaniques (RESAL) competent ethic committee.

Following primers were used for genotyping Ocn^{Cre;Dtr/Yfp} and Ocn^{Gfp} mice:

iDTR =
WSS-F:
(SEQ ID NO: 1)
5′-GGCTACTGCTGACTCTCAACATT-3′;
DTR-R:
(SEQ ID NO: 2)
TCATGGTGGCGAATTCGAT
Cre =
OcnCre-F:
(SEQ ID NO: 3)
CAA ATA GCC CTG GCA GAT TC;
OcnCre-R:
(SEQ ID NO: 4)
TGA TAC AAG GGA CAT CTT CC
GFP (Jackson Laboratory) =
oIMR0872:
(SEQ ID NO: 5)
TTC ATC TGC ACC ACC G;
oIMR1416:
(SEQ ID NO: 6)
TTG AAG AAG ATG GTG CG

Tumor Models

Adenovirus-Cre (AdCre) was delivered intratracheally (i.t.) to KP mice as previously described (20, 21). Mice were analyzed for bone or tumor phenotypes 12-14 weeks post-tumor initiation. Tumor burden was scored by measuring post-mortem lung weight and by histological analyses of lung tissue using hematoxylin and eosin (H&E) stainings. For some experiments micro-computed tomography (CT) was used to monitor tumor burden in the lung. The lung adenocarcinoma cell line KP1.9 was used to induce lung tumors in male wild-type C57BL/6, Ocn^{Cre;Dtr} or Ocn^{Cre;Yfp} mice via intravenous (i.v.) tail vein injection (0.25×10⁶ cells in 100 μl PBS). Male mice with KP1.9 tumors were typically euthanized between 28-41 days after tumor cell injection. Cells of the Lewis lung cancer line (LLC, 1.5×10⁶ cells in 150 μl PBS) were injected i.v. into wild-type C57BL/6 mice and the mice were euthanized 32 days post-tumor cell injection. Diphtheria toxin (DT) was used to deplete Ocn⁺ cells in Ocn^{Cre;Dtr} and Ocn^{Cre;Dtr/Yfp} mice; for the detailed depletion protocol see section: In vivo osteoblast depletion.

Cell Lines

The KP1.9 cell line, derived from lung tumor nodules of a C57BL/6 KP mouse, was provided by Dr. Zippelius (University Hospital Basel, Switzerland). GFP-positive KP1.9 cells (KP-GFP cell line) were established in the laboratory. The LLC cell line was obtained from ATCC and ST2 cells were provided by Marc Wein (MGH). All cell lines were maintained in Iscove's DMEM media supplemented with 10% fetal bovine serum (FBS) and 1% penicillin/streptomycin.

Patient Bone Density Measurements

The study was conducted using IRB approval (2016P000394/MGH) and complied with HIPAA guidelines with exemption status for individual informed consent. A retrospective search was performed to identify patients with non-small cell lung cancer (NSCLC) who were KRAS positive and KRAS negative, and who had undergone non-contrast chest CT prior to therapy at MGH between 2011 and 2017. Patients with osseous metastases, paraneoplastic syndrome or therapy prior to or at time of chest CT were excluded. Control subjects (referred to as control patients) who had undergone non-contrast chest CT using the same imaging protocols as the patient group were identified and 1:1 matched for sex, age within ±2 years, BMI within ±2 kg/m², and smoking (pack-years). Potential controls with active malignancy, significant chronic illness or medication use known to affect bone metabolism were excluded. Trabecular bone density was determined from non-contrast chest CT (16- or 64-MDCT scanner Biograph 16 or 64, Siemens Healthcare; or Discovery CT750HD, GE Healthcare) using an axial slice thickness of 2.5 or 5 mm, 120 kVp and 11-40 mAs. Scans were then reviewed offline on an IMPAX workstation (AGFA Diagnostic Software, version 4, Afga). Circular regions of interest (exemplarily shown in FIG. 2.1F) within trabecular bone of the T6, T8, T10 and T12 vertebral bodies were placed manually, avoiding cortical bone and posterior veins. The mean trabecular bone density of each vertebral body in Hounsfield Units (HU) was determined and an average thoracic trabecular bone density of the four vertebral bodies was calculated.

Fluorescence Molecular Tomography (FMT)

OsteoSense-750EX (18) was injected retro-orbitally (4 nmol/100 μl, Perkin Elmer) according to manufacturer's instructions. The mice (for in vivo study) or cleaned bones (for ex vivo investigation) were imaged with FMT no earlier than 4 h and no later than 24 h post OsteoSense injection using an FMT imaging system (VisEn Medical). For in vivo imaging, hair from hind legs and lower abdomen were removed by shaving and chemical depilation. Mice were anaesthetized using isoflurane during the entire scanning procedure. The positioning of the mice relative to the detector were kept consistent throughout the experiments and groups. Detected OsteoSense signal in the femoral-tibial joint (region of interest, ROI) was analyzed using TrueQuant software and normalized against age and sex-matched control values.

Micro-Computed Tomography (CT) for Lung Tumor Measurements

Lung tumor volumes were received through repeated CT measurements and pre-versus post-treatment measurements calculated. Mice were anaesthetized using isoflurane during the entire scanning procedure. Lungs were imaged with a CT (Quantum FX, PerkinElmer) at a 50 μm voxel size, with retrospective respiratory gating. Individual tumor volumes were measured and calculated using the Analyze software (PerkinElmer).

Micro-Computed Tomography (CT) for Bone Microarchitecture

Femurs from KP 1.9 tumor-bearing versus age-and sex matched tumor-free controls were dissected out, cleaned, fixed in 10% formalin for 24 h, washed in PBS and transferred to 70% ethanol (EtOH) prior to CT analysis. Trabecular bone microarchitecture and cortical bone morphology in the distal femoral metaphysis and mid-diaphysis, respectively, were quantified using a high-resolution desktop micro-tomographic imaging system (.iCT40, Scanco Medical AG). The scans were performed using the following settings: 10 μm³ isotropic voxel size, 70 kVp peak x-ray tube intensity, 114 mA x-ray tube current, 200 ms integration time, and were subjected to Gaussian filtration and segmentation. Image acquisition and analysis protocols were performed according to μCT guidelines for the assessment of bone microstructure in rodents (65). Trabecular bone was analyzed in a region (1500 μm; 150 transverse slices) extending proximally from 200 μm above the peak of the distal growth plate. A threshold of 339 mgHA/cm³ was used to segment trabecular bone from soft-tissue and then, trabecular bone volume fraction (BV/TV, %), trabecular thickness (Tb.Th, mm), trabecular number (Tb.N, 1/mm), trabecular separation (Tb.Sp, mm), and trabecular bone mineral density (Tb.BMD, mgHA/ccm) were measured using the Scanco Evaluation program trabecular morphology script. Cortical bone was evaluated in a 500 m long (50 transverse slices) region at the femoral mid-diaphysis and was segmented using a threshold of 700 mgHA/cm³ and then analyzed using the Scanco mid-shaft evaluation script to measure total cross-sectional area (Tt.Ar, mm²), cortical bone area (Ct.Ar, mm²), medullary area (Ma.Ar, mm²), bone area fraction (Ct.Ar/Tt.Ar, %), cortical tissue mineral density (Ct.TMD, mgHA/cm³), cortical thickness (Ct.Th, mm), cortical porosity (%), as well as the maximum, minimum and polar moments of inertia (Iₘₐₓ, Iₘᵢₙ, and J, mm⁴).

Histology and Immunohistochemistry (IHC)

For histological analysis of tumor burden in mice, lung tissues and femurs were harvested, formaldehyde-fixed and paraffin-embedded following standard procedures and consecutive sections were prepared. Lung tissue sections were stained with H&E to define tumor tissue areas in the lung as described earlier (21).

IHC on mouse tissue sections was performed as previously described (21). Briefly, mouse lung and bone sections were prepared using a Leica RM2255 rotary microtome (Leica Biosystems), dried at 60° C. for 1 h, dewaxed and rehydrated before heat-induced epitoperetrieval (HIER) prior to immunostaining. The sections were incubated in 10 mM Tris (pH9.0) or 10 mM sodium-citrate (pH6.0) buffered solution containing 0.05% Tween and, depending on the Ab used, if needed heated at 120° C. for 2 min using a pressure cooker. To obtain consistent and reliable staining the LabVision Autostainer 360 (Thermo Fisher Scientific) was used. The sections were pretreated using BLOXALL endogenous enzyme blocking solution (Vector Laboratories) for 10 min to destroy all endogenous peroxidase activity. After blocking with normal goat serum, the sections were incubated with rat anti-mouse Ly-6G (clone 1A8, Biolegend) or anti-mouse osteocalcin (clone M-15, Santa Cruz) monoclonal Abs (mAbs) for 1 h followed by several washes and secondary ImmPRESS polymer detection system (Vector Laboratories) according to the manufacturer's protocol. DAB Quanto (Thermo Fisher Scientific) was applied as substrate and hematoxylin used as counterstain.

Prior to preparation of bone tissue sections, femurs were harvested and cleaned, fixed for 24 h in 10% formalin, washed in PBS and transferred to 70% EtOH. The samples were then decalcified in 14% EDTA for up to two weeks and stored in 70% EtOH until paraffin embedding.

For anti-SiglecF staining (rat anti-mouse Siglec-F mAb, clone E50-2440, BD Pharmingen), IHC on murine lung tissue of KP tumor-bearing or tumor-free mice was performed on frozen tissue sections. Spleen tissue sections were prepared for reference positive-control stainings. Frozen tissue sections were generated as described before (21), air-dried and fixed in acetone (−20° C.) for 10 min. The sections were rehydrated and treated as described above, but without HIER.

For histological evaluation of tumor metastases (femur), 10 regions of interest (n=8) on paraffin-embedded decalcified H&E-stained femur sections were defined at 20× and blindly scored for the presence or absence of tumor cell clusters. Positive control evaluations were done on histological sections from KP tumor-bearing lungs.

For cytospins, SiglecF^{high} neutrophils (CD45⁺ CD11b⁺ Ly-6G⁺ SiglecF⁺), SiglecF^{low} (CD45⁺ CD11b⁺ Ly-6G SiglecF⁻) neutrophils and lung alveolar macrophages (CD45⁺CD11b⁻F4/80⁺ SiglecF⁺ CD11c⁺) were FACS sorted from lung tissue of KP tumor-bearing or tumor-free mice based on marker expression using the following anti-mouse mAbs: CD45 (clone 30-F11, Biolegend), Ly-6G (clone 1A8, Biolegend), CD11b (clone M1/70, BD), SiglecF (clone E50-2440, BD), Ly-6C (clone HK1.4, Biolegend), CD11c (clone N418, eBioscience). Cytospins were performed using a Shandon Cytospin 4 centrifuge (Thermo Fisher Scientific). In detail, 5×10⁴ cells were centrifuged (500 rpm, 3 min) onto Tissue Path Superfrost Plus Gold microscope slides (Thermo Fisher Scientific) and dried overnight at RT. Cytospins were then fixed in 4% formaldehyde-buffered solution and H&E stained following standard procedures.

For all histological sections, image documentation was performed using the NanoZoomer 2.0-RS slide scanner system (Hamamatsu).

Bone Histomorphometry

Bone histomorphometric analysis was performed on femurs from KP1.9 tumor-bearing or age and sex-matched tumor-free controls as previously described (66). In brief, calcein (20 mg/kg; Sigma) and demeclocycline (50 mg/kg, Sigma) were injected at 9 days and 2 days prior to animal euthanasia, respectively. Dissected, cleaned, formalin-fixed (10%, 24 h) femurs were washed in PBS and transferred to 70% EtOH. Fixed non-decalcified femurs were dehydrated (graded ethanol) and subsequently infiltrated and embedded in methylmethacrylate. Longitudinal sections (5 μM) were cut using a microtome (RM2255, Leica) and stained with Goldner Trichrome for measurements of cellular parameters and by the method of von Kossa (67) to evaluate bone mineralization. Dynamic bone parameters were evaluated on unstained sections by measuring the extent and the distance between double labels using the Osteomeasure analyzing system (Osteometrics Inc.). Measurements were made in the area 200 μm below the growth plate. Quantification of bone parameters was done in a blinded manner. The structural, dynamic and cellular parameters were evaluated using standardized guidelines (68).

Confocal Microscopy

Confocal microscopy was performed based on a previously published protocol (69). The mice were injected with OsteoSense retro-orbitally to label bone (<24 h before mice were sacrificed) and with fluorescently conjugated mAbs (anti-CD31 (clone MEC13.3, Biolegend), anti-CD144 (clone BV13, Biolegend), anti-Sca1 (clone D7, eBioscience)) 30 min prior to euthanasia via cardiac perfusion with PBS and subsequent 4% methanol-free paraformaldehyde (Alfa Aesar). After fixation, the femurs or sternums were quickly dissected out, cleaned of tissue and cut for imaging. In brief, sternum marrow was exposed by cutting longitudinally along the bone and subsequently scanned at 10× (3-4 partially overlapping field of views). Femurs were OCT embedded, frozen at −80° C. (>1 h), and marrow tissue was exposed using a cryostat. Z-stack images from femur and sternum were immediately acquired at 2-5 μM steps (Olympus IV100 confocal microscope) and analyzed in FIJI (ImageJ). Non-injected controls or non-fluorescent mice were used as staining controls.

In Vivo Gr-1⁺ Cell Depletion

Twelve-to-fourteen week-old KP mice were infected i.t. with 1500 Cre-active lentiviral units using a protocol described earlier (20). KP mice bearing well-established tumors (identified by μCT) were treated 20 weeks after tumor initiation with anti-Gr-1 mAb (10 mg/kg, clone RB6-8C5, BioXcell) intraperitoneally (i.p.) three times per week for 2 weeks. Neutrophil depletion was validated by tail-vein blood sampling at day 7 followed by flow cytometry analyses of SSC^{hi} Ly-6G⁺ circulating cells. Control mice were injected with IgG control mAb (10 mg/kg, clone 2A3, Jackson Immunoresearch). At the end of the experiment tumor-bearing lungs were collected and single-cell suspensions were obtained using the GentleMACS tissue octo dissociator (Miltenyi) and an enzymatic digestion mix composed of DMEM, 0.02 mg/ml DNAse I (Sigma) and 1 mg/ml collagenase (Sigma) applied for 35 min at 37° C. Cells were washed with medium then resuspended in PBS supplemented by 2% FBS and 0.5 mM EDTA. To obtain single-cell suspensions, cells were passed through 70 m cell strainer. Cell number was determined and 10⁷ cells were used for flow cytometry staining. Cells were first stained with live and dead blue dye (Life Technologies) in PBS containing Fc-Block reagent (Miltenyi) for 20 min at 4° C. After washing, mAb staining (anti-Ly-6G-FITC, clone RB6-8C5; anti-CD11b-BV711, clone M1/70; anti-Ly-6C-AlexaFluor700, clone HK1.4; anti-CD11c-BV450, clone N418 and anti-CD45-PerCP, clone 30-F11; all from Biolegend) was performed on ice or a 4° C. in PBS supplemented with 2% FBS and 0.5 mM EDTA for 15 min. All acquisitions were performed using the LSRII SORP (BD), a 5-laser and 18-detector analyzer at the EPFL Flow Cytometry Core Facility. Data analyses were performed using FlowJo X (FlowJo LLC).

In Vivo Osteoblast Depletion

Ocn^{Cre;Dtr}, Ocn^{Cre;Dtr/Yfp} and control mice lacking either transgene were treated i.p. with DT (100 μl; 20 μg/kg, Sigma-Aldrich) every other day for 9 days with a total of five injections per mouse. In some experiments, osteoblast depletion was performed for 3 consecutive days using DT. For both DT treatment protocols, osteoblast depletion was verified. Body weight was monitored to control for DT-induced toxicity. Osteoblast depletion was verified using histological evaluation of femurs, IHC for osteocalcin and ex vivo whole mount immunofluorescence of Ocn^{Cre;Dtr/Yfp} mice. Performing in vitro DT titration studies and in vivo cellular staining using flow cytometry, it was ensured that the DT concentration used did not affect the viability of hematopoietic cells in this murine model.

In Vivo NK Cell Depletion

NK cells were depleted in tumor-bearing Ocn^{Cre;Dtr} or control mice performing i.p. injections of anti-NK1.1 Ab (clone PK136, BioXcell, 200 μg/mouse, i.p.) every fourth day. The detailed treatment schema is outlined in FIG. S11A. NK cell mAb depletion in osteoblast-reduced tumor-bearing mice was evaluated using flow cytometry and was efficient in substantially decreasing lung NK cells (detected by CD49b and NKp46 double staining since the NK1.1 epitope may be masked by the depleting mAb).

In Vitro Assay to Test Potential DT-Mediated Direct Effects on Hematopoietic Cells

Splenocytes from WT or Ocn^{Cre;Dtr} mice were harvested by gently meshing a spleen through a 40 m filter. The cells were washed, plated in medium (RPMI, 10% FBS, 1% P/S) and treated with 0, 1, 10, 100, 1000 ng/ml of DT. Cells were harvested after 20 h of incubation at 37° C. and stained with mAbs for flow cytometry, see section on flow cytometry for staining procedure.

Parabiosis

In some experiments, parabiosis was used to study the contribution of circulating cells to osteoblast-controlled tumor-infiltrating immune cells. The experimental procedure was performed as previously described (70). In brief, one week post-tumor injection, lung tumor-bearing Ocn^{Cre;Dtr} mice were parabiosed to Ocn^{Cre;Dtr} or control mice (lacking either transgene). Both types of parabionts were treated with DT following the procedure described in section: In vivo osteoblast depletion.

Neutrophil Single Cell RNAseq

Single-cell RNA sequencing (scRNA-Seq) data were obtained from CD45⁺ cells collected from either tumor-free or KP tumor-bearing lungs from two independent experimental replicates using droplet microfluidic barcoding technology (inDrops) as previously described (71, 72). To identify single cell expression profiles corresponding to neutrophils among other CD45⁺ cells, a naive Bayes classifier utilizing immune cell gene expression profiles from the Immgen consortium was applied (73). Data from the isolated cell transcriptomes is not shown herein. Neutrophil transcriptomes were further divided into SiglecF^{high} and SiglecF^{low} cells as follows. Due to the limited sensitivity of scRNA-Seq at the single-cell level, cells were classified based on a composite SiglecF expression score S, among granulocytes (n=6,020 cells). For each single cell k, Sₖ=(Xₖ−Yₖ), where Xₖ=Σᵢ₌₀⁵⁰r_{k,i} and r_{k,i} is defined as the percentile gene expression (dense ranking) of cell k for gene i, for the 50 most correlated genes to SiglecF (Spearman correlation), and Yₖ=Σ_{i=N=49}^{N}r_{k,i} is the corresponding sum of percentiles of the 50 most anticorrelated genes to SiglecF (Table 1). As anticipated from FACS data, the distribution of granulocytes by Siglecf expression score was bimodal in tumor, with SiglecF^{low} cells overlapping with healthy granulocytes. By visual inspection of FIG. 24A, a threshold of −7 was set to separate between SiglecF^{high} and SiglecF^{low} granulocytes in tumor tissue.

For differential gene expression (DGE) analysis of healthy, tumor SiglecF^{low}, and tumor SiglecF^{high} granulocyte populations, a parameter-free permutation-based test to calculate p-values was used, with the difference in means as the test statistic. For multiple hypothesis testing with a false discovery rate of 5% using the Benjamini-Hochberg procedure was accounted for (74). To be considered for differential gene expression analysis, genes had to be expressed at least by 5% of cells in at least one of the two groups of cells compared. Significantly differentially expressed genes with an absolute fold-change of 2 were selected for further analysis. Genes judged as significant, for example by the permutation test but with a p-value less than the specified accuracy of the permutation test were assigned an approximate p value using a t-test assuming unequal variances for representation on volcano plots.

For gene set enrichment analysis (75, 76), the same pre-filtering as for DGE analysis was performed: only genes expressed by at least 5% of cells in at least on the two groups in a comparison were considered. The GSEA PreRanked tool (75, 76) was then used on genes ranked by log 2 (fold-change) and considered gene sets that were enriched based on an FDR of 25%.

Osteoblast Low-Input Bulk RNAseq

KP-Ocn^{Gfp} mice were infected with Ad-Cre i.t. and euthanized when high tumor burden was detectable (at 14 weeks post AdCre). All bones were harvested, cleaned and pooled from each single mouse. The bones were crushed gently and the released cells were collected (fraction 1). Red blood cells were lysed using ACK buffer (Lonza) and cells were depleted of mature cells using the lin-depletion kit (Stem cell technologies). In parallel, the bone fragments (fraction 2) were cut finely with scissors, filtered through a 70 uM cell strainer (BD), digested for 1 h at 37° C. (0.25% collagenase type I (Worthington Biochemical Corporation) in FBS), washed and pooled with fraction 1. Ocn-expressing cells were FACS sorted (FACSAria) based on the following parameters: Lin⁻CD45⁻CD31⁻Ter119⁻GFP⁺. Approximately 2000 cells were sorted per mouse into Trizol and frozen at −80° C. RNA was isolated using a Trizol extraction protocol according to the Immgen standard, which can be found on the world wide web at the website, www.immgen.org/Protocols/Total %20RNA %20Extraction %20with %20Trizol.pdf.

Libraries were made following the protocol by Meredith et al. (77). RNA was reverse transcribed using ArrayScript (Ambion) using a specific primer containing T7 promoter, the 5′ TruSeq Illumina adapter, a 8-positions with random nucleotide assignment as a unique molecular identifier (UMI), and a oligo-dT sequence. Second-strand synthesis was performed using the mRNA Second strand synthesis module (NEBNext #E6111L). After cDNA size selection using AMPure XP beads (0.8× and 1×, BeckmanCoulter-A63987), the product was amplified via in vitro transcription (MEGAshortscript, Invitrogen) for 12 hours and then fragmented (Magnesium RNA Fragmentation Module, New England Biolabs). 3′ indexing adaptor was ligated (truncated T4 RNA ligase 2-Enzymatics), reverse-transcribed (Superscript II, Invitrogen), and amplified by PCR for 18 cycles (HiFi hotstart PCR kit, Kapa). cDNA cleanup and size selection were performed on AMPure XP beads. Libraries were quantitated by BioAnalyzer using the Agilent High Sensitivity DNA Kit (Agilent 5067-4626) and qPCR using Kapa library quantification kits, and sequenced on a MiSeq (nano kit) and HiSeq 2500 (rapid mode).

Raw sequencing reads were processed using custom scripts. Read 1 contains the transcript sequence, Read 2 the UMIs. Raw reads were first trimmed using the FASTX-Tollkit v0.0.13 (fastx_trimmer -Q 33) (78). Read 2 was trimmed in order to extract the UMI (5-12 bp), and Read 1 was trimmed to 30 bp eliminate a potential oligo-dT sequence. Reads were filtered for quality (more than 80% of the sequence having a Sanger Phred+33 quality score >33) using fastq_quality_filter -v -Q 33 -q 20 -p 80. Mapping was performed with Tophat2 to the mm 10 mouse transcriptome (79) keeping the strand information with the following options: tophat -p 2--library-type fr-firststrand --read- mismatches 5 --read-gap-length 5 --read-edit-dist 5 --nocoverage-search --segment- length 15 --transcriptome-index. Reads mapping at multiple positions were discarded using samtools flag 256 (80). Duplicated mapping reads were filtered out using the UMIs with custom R scripts as follows. Reads were first assigned to genes. For each gene, only reads with distinct UMIs were kept. To take into account mutations in UMIs, distinct UMIs but with a Hamming distance of 1 were also collapsed to 1 read. Samples were normalized with DESeq using the estimateSizeFactors function (81). Multiplot studio was used to define differentially expressed genes in Ocn⁺ cells between tumor-bearing and tumor free mice (p<0.05). Osteoblast RNAseq data has been deposited under accession number GSE104294.

Survival Analysis of Lung Adenocarcinoma Patients

Analyses were performed using tumor microarray data and survival outcome in lung adenocarcinoma patients. Raw microarray CEL files along with patient annotations were obtained from two sources: 1) GSE68465 (34) and 2) on the world wide web at the address(35). CEL files from individual patients were converted into a single expression matrix using ExpressionFileCreator (v12.3, method=MAS5), followed by quantile normalization using the array NCI_U133A_61L as a reference as described before (34). Probes were collapsed to gene symbols by selecting the probe with maximum mean expression after excluding probes mapping to multiple gene symbols (82). From the list of differentially expressed genes (data not shown) between T-SiglecF^{high} and T-SiglecF^{low} neutrophils, genes with a minimum expression of 50 transcripts per million and >5 times higher expression in T-SiglecF^{high} neutrophils were selected. The resulting 305 mouse genes were mapped to human orthologs using the HCOP tool, which can be found on the world wide web at the address http://www.genenames.org/cgi-bin/hcop, including orthology predictions from Ensembl, NCBI, HGNC, Panther, HomoloGene, OrthoDB, OrthoMCL, OMA, PhylomeDB, TreeFam, Inparanoid, EggNOG. All orthologs were included for mouse genes mapping to multiple human genes. The conversion yielded 302 human orthologs (Table 2). Using human patient microarray data, each patient was attributed a “T-SiglecF^{high} neutrophil signature” value, defined as the sum rank transformed expression of the 302 human orthologs of genes enriched in T-SiglecF^{high} neutrophils in mouse. Here, rank transformation refers to the process by which the expression of gene i in patient j in the microarray data is replaced with the rank for patient j among other patients based on the expression of i (dense ranking). The signature was rescaled to have values from 0 to 1. Cox regression analysis was performed using the T-SiglecF^{high} neutrophil signature, sex, age, T stage, and N stage as predictor variables. Other sample characteristics, which were not documented for a fraction of patients, were used as strata; these included M stage, source of data, histological grade, smoking history, treatment with adjuvant chemotherapy and radiotherapy, tumor relapse, and positive surgical margin (data not shown). All predictor variables satisfied the proportional hazards assumption as validated by a Schoenfeld residual tests (cox.zph function in R). To further validate the statistically significant p-value returned by the Cox Hazard test (p value=0.0017), 302 genes present in the microarray data were randomly sampled, and recorded the number of times a Cox p-value smaller than the one observed was obtained. If the Cox Hazard model is accurate, it would expect approximately 0.17% of random trials to give the observed p-value or less. 7 out 1000 random samplings (0.7%) yielded a Cox p-value <0.0017, indicating a slight underestimate of the p-value by the Cox Hazard model, but nonetheless allowing to reject the null hypothesis of the gene selection being random with p<0.01. The T-SiglecF^{low} gene signature was defined in an analogous way, using the same number (n=302) of human orthologs of genes most enriched in T-SiglecF^{low} neutrophils. T-SiglecF^{low} neutrophil gene signature showed no significant association with survival. For Kaplan-Meier plots, survival data of top 25% and bottom 25% SiglecF^{high} signature expressers was used. Survival analysis was performed using the “survival” package in R (83) and “Lifelines” package in Python (84).

In Vivo Cell Fate Mapping

To track the progeny of hematopoietic precursors in tumor-bearing control or Ocn depleted mice, cell fate mapping experiments were performed. Bead enrichment (Miltenyi) was used followed by FACS-based sorting of live lineage negative congenic CD45.1 cKIT⁺ (CD117) cells (here lineage=B220, CD19, Ter1119, CD11c, CD11b, NK1.1, CD49b, CD127, Ly-6G, CD90.2). The purity of the sorted CD45.1⁺ cKIT⁺ cells was above 95%. 2.5×10⁵ cells were injected i.v. into tumor-bearing control or Ocn depleted mice (both CD45.2 genotypes) at 29 days post tumor-injection. 7 days post-cKit⁺ cell transfer, lung tumor tissue was harvested and CD45.1⁺ immune cell infiltrates were quantified using flow cytometry. Non-injected biological controls, Fluorescence Minus One (FMO)-staining controls and unstained cells were used to analyze the CD45.1⁺ cell progeny in the tissue.

In Vivo Tumor Cell and Neutrophil Co-Injection Experiment

To investigate whether SiglecF^{high} neutrophils are able to support the growth of tumor cells in vivo, KP-GFP tumor cells were co-injected with different neutrophil subpopulations (T-SiglecF^{high}, T-SiglecF^{low} or H-SiglecF^{low}) intradermally (i.d.) to the flank of C57BL/6 mice. Neutrophils were FACS sorted based on cell surface marker expression (CD45⁺CD11b⁺Ly-6G⁺SiglecF⁺ or SiglecF⁻) from lungs of KP1.9 tumor-bearing or tumor-free mice as detailed in the Flow cytometry methods section. Tumor cells (2×10⁵) and the respective neutrophil population (2×10⁵) were mixed in 50 μl 1×PBS before i.d. injection (1:1 ratio). Tumor growth was recorded over time with a digital caliper and tumor volumes defined as H/6×length×width².

Ex Vivo ROS Activity Assay

Neutrophils were analyzed ex vivo for their reactive oxygen species (ROS) content. Single cell suspensions were generated from KP tumor-bearing lungs or lungs of tumor-free mice as described in the Flow cytometry methods section. Cells were resuspended in HBSS containing 0.1% BSA followed by FACS antibody surface marker staining for 30 min on ice as detailed below. Then cells were washed and resuspended in PBS-EGG buffer (1 mM EDTA, 0.05% gelatin, 0.09% glucose) and 0.5 μM DHR123 probe (Thermo Fisher Scientific) was added for 30 min at 37° C. The reaction was stopped by moving the tubes to ice and washing the cells with PBS-EGG buffer. Cells were resuspended in PBS containing 0.1% BSA and activated rhodamine 123 signal (activated DHR 123) was analyzed in the FITC channel on a LSRII flow cytometer (BD) within 30 min. Hydrogen peroxide added to cells served as a positive control.

In Vitro Macrophage Differentiation Experiment

Monocytes and neutrophil were co-cultured to investigate if neutrophils can help to mature macrophages from their monocytic precursors. Neutrophils were FACS sorted based on cell surface marker expression (CD45⁺CD1b⁺Ly-6G⁺SiglecF⁺or SiglecF⁻) from lungs of KP 1.9 tumor-bearing (T-SiglecF^{low} or T-SiglecF^{high}) or tumor-free mice (H-SiglecF^{low}) as detailed in the Flow cytometry methods section. Murine spleens were harvested from tumor-bearing mice and were used to enrich for monocytes. In detail, spleens were harvested, meshed through a 40 μm cell strainer and ACK lysed to remove erythrocytes. Splenic monocytes were enriched through a MACS based negative isolation protocol by incubating single cells with PE conjugated Abs specific for CD90.2, CD3, B220, CD19 and Ly-6G followed by anti-PE MACS beads. Both incubation steps were performed for 20 min on ice. The negative isolation resulted in a 20-fold enrichment of monocytes based on flow cytometry measurements. This population likely also include myeloid precursors since these accumulate in spleens of tumor-bearing mice (8). For the co-culture, 4×10⁴ monocytes and 8×10⁴ neutrophils were incubated in Iscove's DMEM media supplemented with 10% FBS and 1% penicillin/streptomycin in 96-well cell culture plates for 6 days. Monocytes only and monocytes together with CSF-1 (1 μl/ml) were used as controls. Cells were removed from the plate and investigated by flow cytometry for F4/80 and CD11b expression in order to assess myeloid cell differentiation.

In Vitro Alkaline Phosphatase Assay for Osteoblastic Colony Formation

To study whether tumor derived circulating factors can affect the osteogenic potential, osteoblastic colony formation was investigated after addition of serum pooled from individual mice that were either tumor-bearing or tumor-free. In some experiments tumor-free serum with or without sRAGE was added. Long bones (femur and tibia) and vertebrae of tumor-free C57BL/6 mice were harvested and flushed. Single cell suspensions were generated using 70 m cell strainer and red blood cells removed in a ACK lysis step. Cells were counted and resuspended (4×10⁷/ml) in osteogenic medium (DMEM supplemented with 10% FBS and 1% penicillin/streptomycin, 10 mM B-glycerophosphate and 50 μg/ml ascorbic acid). 4×10⁶ cells were transferred to 6 well cell culture plates and 200 μl serum of tumor-bearing or tumor-free mice added. Medium was refreshed every second day and non-adherent cells were removed. Cells were fixed after 9-10 days by adding 4% PFA for 8 min followed by 2 washing steps with H₂O and the alkaline phosphate substrate reaction (B5655, Sigma) according to manufactures procedure. As counter stain, Nucleofast Red (Polysciences, Inc) was used. The number of ALP positive colonies was evaluated by 2-3 independent persons in a blinded manner.

In Vitro Co-Culture of Hematopoietic Precursors and Bone Marrow Stromal Cells +/−sRAGE

To test whether sRAGE altered neutrophil maturation from hematopoietic precursors via stromal cells, co-culture experiments were performed. In detail, 1×10³ ST2 cells per well were cultured in 96 well plates for two days. Then, bone marrow was harvested from tumor-free mice and depleted of differentiated cells using negative MACS bead separation (Abs specific for B220, CD19, Ter119, CD11c, CD11b, NK1.1, Dx5, CD127, Ly-6G and CD90.2 were utilized). The flow-through was collected. The following conditions were tested: ST2 cells, with or without 1×10⁴ lineage negative bone marrow cells, and with increased doses of sRAGE (namely: no sRAGE, 0.01 μg/ml, 0.1 μg/ml, 1 μg/ml). Neutrophil maturation was evaluated after three days by staining for CD11b, Ly-6G and CXCR2 surface expression using flow cytometry (1).

p53 Recombined PCR for Tumor Cell Detection

Detection of p53 recombined locus (only present in KP tumor cells after exposure to Cre recombinase) was used to survey bone and marrow tissues for KP tumor cell metastases. In brief, DNA was extracted from bone marrow or calvarial bone (after digestion) using DNeasy blood and tissue kit (Qiagen) according to manufacturer's instructions. KP1.9 tumor cells were used as positive control. Different DNA concentrations from KP1.9 tumor cells were used to determine PCR detection limit to <10 cells (with the estimate of ˜6 pg DNA/cell). DNA was isolated from Gel PCR products from a primary PCR run. A second PCR amplification run on these DNA samples was performed to detect low levels of DNA in the isolated tissues. The following primers were used: A: 5′ CAC AAA AAC AGG TTA AAC CCA G 3′; B: 5′ AGC ACA TAG GAG GCA GAG AC 3′; C: 5′ GAA GAC AGA AAA GGG GAG GG 3′. Following bands were amplified: p53 recombined 1lox: 612 bp, WT band: 288 bp and a background band: 400 bp.

Real-Time PCR for Analysis of Blood Samples

Neutrophils from the blood of KP lung tumor-bearing or tumor-free mice were investigated in order to define whether these cells exhibited transcriptional characteristics of tumor-infiltrating SiglecF^{high} cells outside the tumor microenvironment. Neutrophils were FACS sorted based on surface marker expression (CD45⁺ CD11b⁺ Ly-6G⁺) and RNA was isolated from the sorted cells using the RNeasy Micro Kit (Qiagen) according to manufactures procedures. Afterwards, cDNA was generated utilizing the High Capacity cDNA Reverse Transcription Kit (Applied Biosystems) and Real time PCR assays performed using the TaqMan Fast Advanced MasterMix together with TaqMan probes at the 7500 Fast Real-Time PCR System (Applied Biosystems). 32-Microglobulin was used as a housekeeping gene.

Protein Array

To investigate soluble factors in serum or plasma of KP and KP 1.9 lung tumor-bearing or tumor-free control mice, a membrane-based sandwich immunoassay with 111 different cytokine and chemokine antibodies was performed using the Proteome Profiler Mouse XL Cytokine Array Kit (R&D Systems) according to manufactures procedures. The captured soluble factors were visualized in duplicate using a chemiluminescent detection readout with an exposure time of 3 min. The signal intensity from each protein spot on the array was quantified using the Microarray_Profile plugin in FIJI (http://www.optinav.info/MicroArray_Profile.htm). The intensity was normalized against six reference spots on each array. From two independent experiments, fold change of tumor versus tumor-free soluble factors was calculated and the resulting values presented in a heat map. Factors below detection level and that failed to alter reproducibly were excluded from further analysis.

sRAGE ELISA

Mouse sRAGE levels in the serum of KP 1.9 tumor-bearing or tumor-free mice were quantified using ELISA according to manufacturer's instructions (MRGOO, R&D systems). OD values were measured at 450 and 570 nm (reference value) using a Tecan microplate reader. Murine blood was harvested and transferred to BD Microtainer tubes, incubated at room temperature for 30 min, spun at 1000 g for 15 min and the serum was stored at −80° C. until ELISA analysis. The concentration of sRAGE in serum samples, investigated in duplicate, was calculated by extrapolating values of a standard curve following manufactures guidelines.

Flow Cytometry

Single cell suspensions were obtained from lung tumors, bone marrow, spleen and bone tissue. The respective tissues and isolated single cell fractions were kept on ice for all steps if not stated otherwise. Tumor tissue was received by dissecting out tumor-bearing lungs. Small tissue pieces were generated using scissors and digested (RPMI containing 0.2 mg/ml collagenase type I, Worthington Biochemical Corporation) for 1 h at 37° C. while shaking. Femurs and for some experiments tibias were harvested, cleaned and the bone marrow flushed out using cold staining buffer (PBS containing 0.5% BSA and 2 mM EDTA). Digested lung tissue and harvested bone marrow were gently meshed through 40 μM cell strainers using a plunger. Spleens were harvested and also meshed through 40 μM cell strainer as described before. Red blood cells were removed using 1 ml ACK lysis buffer (Lonza) per cell pellet for 1 min (for lung cells) or 2 min (for spleen cells) and the reaction was stopped with RPMI media. In some experiments blood was collected from the cheek or if mice were euthanized via cardiac puncture and directly treated with 5 ml ACK lysis buffer for 5 min to remove red blood cells. The resulting single-cell suspensions were washed and resuspended in staining buffer. In order to investigate bone cells by flow cytometry, in general, long bones were harvested, cleaned and crushed gently and the released cells were collected (fraction 1) and lysed with ACK lysis buffer. In parallel, the bone fragments (fraction 2) were cut into small pieces with scissors, filtered through 70 μM cell strainer, digested (PBS containing 20% FBS and 0.25% collagenase type I) for 1 h at 37° C., washed and finally pooled with the cells derived from fraction 1.

Single cell suspensions were incubated with FcBlock (clone 93, Biolegend) for 15 min at 4° C., followed by staining with fluorescent conjugated Abs for 45 min at 4° C. The cells were washed with staining buffer and analyzed on a LSRII flow cytometer (BD). 7-aminoactinomycin (7AAD, Sigma) positivity was used to exclude dead cells. Flow Cytometry Absolute Count Standard (Bangs Laboratories) were used to quantify circulating neutrophils.

Following cell populations were identified based on cell marker expression: Ocn⁺ cells (Lin⁻ CD45⁻ CD31⁻Ter119⁻YFP⁺), neutrophils (CD45⁺ CD11b⁺ Ly-6G⁺), SiglecF^{high} neutrophils (CD45 CD11b⁺ Ly-6G⁺ SiglecF^{high}), SiglecF^{low} neutrophils (CD45⁺ CD11b⁺ Ly-6G⁺SiglecF^{low}), monocytes (CD45⁺ CD11b⁺ Ly-6G⁻ Ly-6C^{high}), CD11b⁻ alveolar macrophages (CD45⁺ CD11b⁺ F4/80⁺ SiglecF⁺ CD11c⁺), CD11b⁺ macrophage-like cells (CD45⁺ CD11b⁺ Ly6G⁻ Ly6C⁻), T cells (CD45⁺ CD3⁺CD4⁺ or CD8⁺), B cells (CD45⁺ B220⁺ CD19⁺), NK cells (CD45⁺ CD49b⁺ NK1.1⁺ or CD45⁺ CD49b⁺ NKp46⁺).

The lineage (Lin) Ab mix contained the following Abs unless otherwise noted: B220, CD19, Ter119, CD11c, CD11b, NK1.1, CD49b, CD127, Ly-6G, CD90.2.

Following Abs were purchased from BD if not mentioned differently: B220 (553089, clone RA3-6B2); CD19 (553786, clone 1D3); Ter119 (553673, clone TER-119); CD11c (12-0114-83, clone N418, eBioscience); CD11b (557397, clone M1/70); NK1.1 (553165, 550627, clone PK136); CD49b (553858, clone DX5); CD127 (12-1271-82, clone A7R34, eBioscience); Ly-6G (551461, 560599, clone 1A8); CD90.2 (553006, clone 53-2.1); SiglecF (564514, clone E50-2440); CD4 (557956, clone RM4-5), Biolegend: CD117 (105812, clone 2B8); F4/80 (123115, clone BM8); CD45.1 (110738, clone A20); CD45.2 (109831, clone 104); CD3e (100306, clone 145-2C11); CD8 (100725, clone 53.-6.7); CD19 (115530, clone 6D5); CD11c (117333, clone N418); Nkp46 (137619, clone 29A1.4); CD45 (103126, clone 30-F11); CXCR2/CD182 (149303, clone SA044G4) or R&D Systems: CLEC5a/MDL-1 (FAB1639P, clone 226402).

Statistical Methods

Unpaired t-test was used to compare two groups. Multiple t-test was performed to compare several cell populations between two groups and false discovery rate was accounted for using the Benjamini-Hochberg-Yekutieli procedure with Q=1%. One-way or Two-way ANOVA with subsequent post-hoc analysis was done to compare three or more groups. GraphPad Prism was used to test for statistical significance except for when noted. Matlab and Python were used for scRNAseq analysis, corresponding statistical testing is described above in section ‘Neutrophil single cell RNAseq’. Python was used for patient survival analysis as detailed in section ‘Bioinformatical analyses of lung adenocarcinoma patients’. *p<0.05, **p<0.01, ***p<0.001, ****p<0.0001, n.s. not significant.

### REFERENCES

1. S. B. Coffelt, M. D. Wellenstein, K. E. de Visser, Nat Rev Cancer 16, 431 (2016).
2. D. Hanahan, L. M. Coussens, Cancer Cell 21, 309 (2012).
3. D. I. Gabrilovich, S. Ostrand-Rosenberg, V. Bronte, Nat Rev Immunol 12, 253 (2012).
4. C. Engblom, C. Pfirschke, M. J. Pittet, Nat Rev Cancer 16, 447 (2016).
5. S. S. McAllister, R. A. Weinberg, Nat Cell Biol 16, 717 (2014).
6. A. J. Casbon et al., Proc Natl Acad Sci USA 112, E566 (2015).
7. L. J. Bayne et al., Cancer Cell 21, 822 (2012).
8. V. Cortez-Retamozo et al., Proc Natl Acad Sci USA 109, 2491 (2012).
9. W. C. Wu et al., Proc Natl Acad Sci USA 111, 4221 (2014).
10. A. J. Templeton et al., J Natl Cancer Inst 106, dju124 (2014).
11. L. Jiang et al., Oncotarget 6, 9542 (2015).
12. S. H. Huang et al., Cancer 121, 545 (2015).
13. J. Hoggatt, Y. Kfoury, D. T. Scadden, Annu Rev Pathol 11, 555 (2016).
14. S. J. Morrison, D. T. Scadden, Nature 505, 327 (2014).
15. A. Mendelson, P. S. Frenette, Nat Med 20, 833 (2014).
16. M. R. Reagan, C. J. Rosen, Nat Rev Rheumatol 12, 154 (2016).
17. L. M. Calvi et al., Nature 425, 841 (2003).
18. A. Zaheer et al., Nat Biotechnol 19, 1148 (2001).
19. R. Weissleder, M. J. Pittet, Nature 452, 580 (2008).
20. M. DuPage, A. L. Dooley, T. Jacks, Nat Protoc 4, 1064 (2009).
21. C. Pfirschke et al., Immunity 44, 343 (2016).
22. G. Karsenty, H. M. Kronenberg, C. Settembre, Annu Rev Cell Dev Biol 25, 629 (2009).
23. J. Y. Wu, D. T. Scadden, H. M. Kronenberg, J Bone Miner Res 24, 759 (2009).
24. M. Weinreb, D. Shinar, G. A. Rodan, J Bone Miner Res 5, 831 (1990).
25. I. Bilic-Curcic et al., Genesis 43, 87 (2005).
26. A. Bozec et al., J Cell Biol 190, 1093 (2010).
27. A. Bozec et al., J Cell Sci 126, 5432 (2013).
28. B. M. Abdallah et al., J Bone Miner Res 26, 1457 (2011).
29. K. Watari et al., Sci Rep 6, 19470 (2016).
30. J. R. Cubillos-Ruiz et al., Cell 161, 1527 (2015).
31. Z. Ang, J. Z. Er, J. L. Ding, Sci Rep 5, 8134 (2015).
32. J. Schmielau, O. J. Finn, Cancer Res 61, 4756 (2001).
33. S. Kusmartsev, Y. Nefedova, D. Yoder, D. I. Gabrilovich, J Immunol 172, 989 (2004).
34. C. C. F. T. M. C. O. L. A. Director's et al., Nat Med 14, 822 (2008).
35. D. X. Nguyen et al., Cell 138, 51 (2009).
36. A. M. Schmidt, Vascul Pharmacol 72, 1 (2015).
37. Z. Zhou, W. C. Xiong, Front Biosci (Schol Ed) 3, 768 (2011).
38. E. Lalla et al., J Clin Invest 105, 1117 (2000).
39. N. Mercer, H. Ahmed, S. B. Etcheverry, G. R. Vasta, A. M. Cortizo, Mol Cell Biochem 306, 87 (2007).
40. H. Nozawa, C. Chiu, D. Hanahan, Proc Natl Acad Sci USA 103, 12493 (2006).
41. F. Shojaei et al., Nature 450, 825 (2007).
42. M. Kowanetz et al., Proc Natl Acad Sci USA 107, 21248 (2010).
43. Z. G. Fridlender et al., Cancer Cell 16, 183 (2009).
44. S. K. Wculek, I. Malanchi, Nature 528, 413 (2015).
45. S. B. Coffelt et al., Nature 522, 345 (2015).
46. A. J. Gentles et al., Nat Med 21, 938 (2015).
47. D. I. Gabrilovich, Cancer Immunol Res 5, 3 (2017).
48. D. Visnjic et al., J Bone Miner Res 16, 2222 (2001).
49. M. Zhang et al., J Biol Chem 277, 44005 (2002).
50. D. Visnjic et al., Blood 103, 3258 (2004).
51. A. Greenbaum et al., Nature 495, 227 (2013).
52. L. Ding, S. J. Morrison, Nature 495, 231 (2013).
53. J. Y. Wu et al., Proc Natl Acad Sci USA 105, 16976 (2008).
54. J. Zhu et al., Blood 109, 3706 (2007).
55. V. W. Yu et al., J Exp Med 212, 759 (2015).
56. K. Fulzele et al., Blood 121, 930 (2013).
57. M. H. Raaijmakers et al., Nature 464, 852 (2010).
58. A. Kode et al., Nature 506, 240 (2014).
59. A. E. Broadus et al., N Engl J Med 319, 556 (1988).
60. R. Coleman et al., Ann Oncol 25 Suppl 3, iii124 (2014).
61. R. Rizzoli et al., Osteoporos Int 24, 2929 (2013).
62. E. Pompe et al., Eur Radiol 25, 283 (2015).
63. S. Srinivas et al., BMC Dev Biol 1, 4 (2001).
64. T. Buch et al., Nat Methods 2, 419 (2005).
65. M. L. Bouxsein et al., J Bone Miner Res 25, 1468 (2010).
66. M. N. Wein et al., Nat Commun 7, 13176 (2016).
67. E. S. Liu et al., J Bone Miner Res 31, 929 (2016).
68. D. W. Dempster et al., J Bone Miner Res 28, 2 (2013).
69. G. Courties et al., Circ Res 116, 407 (2015).
70. F. Pucci et al., Science 352, 242 (2016).
71. A. M. Klein et al., Cell 161, 1187 (2015).
72. R. Zilionis et al., Nat Protoc 12, 44 (2017).
73. T. S. Heng, M. W. Painter, G. P. C. Immunological, Nat Immunol 9, 1091 (2008).
74. Y. Benjamini, Y. Hochberg, Journal of the Royal Statistical Society 57, 289 (1995).
75. A. Subramanian et al., Proc Natl Acad Sci USA 102, 15545 (2005).
76. V. K. Mootha et al., Nat Genet 34, 267 (2003).
77. M. Meredith, D. Zemmour, D. Mathis, C. Benoist, NatI mmunol 16, 942 (2015).
78. A. Gordon, G. J. Hannon, (2010).
79. D. Kim et al., Genome Biol 14, R36 (2013).
80. H. Li et al., Bioinformatics 25, 2078 (2009).
81. M. I. Love, W. Huber, S. Anders, Genome Biol 15, 550 (2014).
82. J. A. Miller et al., BMC Bioinformatics 12, 322 (2011).
83. T. Therneau, version 2.38, (2015).
84. C. Davidson-Pilon, (2016).
85. P. J. Collins et al., FASEB J 31, 3084 (2017).

Table 1 shows the 50 genes that are most correlated with the SiglecF signature.

TABLE 1
50 genes most correlated to SiglecF signature.

| Gene Symbol | p-value | r (Spearman correlation coefficient) |
|---|---|---|
| Siglecf | 0.00E+00 | 1 |
| Clec4n | 0.00E+00 | 0.505513 |
| Hexb | 0.00E+00 | 0.476419 |
| Ltc4s | 1.56E−307 | 0.456352 |
| mt-Nd1 | 1.10E−254 | 0.419052 |
| Scimp | 7.83E−253 | 0.417656 |
| mt-Nd2 | 2.83E−246 | 0.412657 |
| B4galt1 | 2.43E−237 | 0.405708 |
| mt-Nd3 | 2.95E−234 | 0.40327 |
| Ppia | 3.19E−226 | 0.396821 |
| Cdkn1a | 6.52E−225 | 0.395756 |
| Bhlhe40 | 2.13E−217 | 0.389568 |
| mt-Atp6 | 2.44E−217 | 0.389519 |
| Xbp1 | 1.09E−198 | 0.373558 |
| AA467197 | 2.58E−196 | 0.371458 |
| mt-Cytb | 3.30E−195 | 0.370475 |
| Ptma | 1.38E−186 | 0.362695 |
| Id2 | 3.47E−179 | 0.355837 |
| Rgs10 | 4.09E−177 | 0.353888 |
| mt-Nd4 | 2.38E−176 | 0.353164 |
| Gm11410 | 2.66E−156 | 0.333481 |
| Rps28 | 1.99E−154 | 0.331567 |
| Itgax | 1.01E−148 | 0.325651 |
| Gpr171 | 1.01E−148 | 0.325651 |
| Entpd3 | 4.16E−147 | 0.32395 |
| mt-Co3 | 3.71E−146 | 0.322944 |
| Mrpl52 | 1.62E−139 | 0.315813 |
| Bcl2a1b | 4.00E−139 | 0.315384 |
| Ms4a6d | 3.08E−134 | 0.309998 |
| mt-Nd5 | 8.01E−134 | 0.309535 |
| mt-Co2 | 3.24E−133 | 0.308856 |
| Id1 | 8.96E−132 | 0.307239 |
| Ffar2 | 2.56E−125 | 0.299862 |
| Atp6v0c | 8.52E−124 | 0.298092 |
| Cish | 8.54E−122 | 0.295745 |
| Rps18 | 1.53E−119 | 0.293076 |
| Pald1 | 2.16E−118 | 0.291701 |
| Runx1 | 3.56E−118 | 0.291441 |
| RP23-27H19.7 | 3.04E−117 | 0.290323 |
| Rps29 | 3.66E−117 | 0.290225 |
| Adgre1 | 1.80E−114 | 0.286959 |
| Rps17 | 6.84E−114 | 0.28625 |
| Clec5a | 4.45E−113 | 0.285251 |
| Ubash3b | 5.10E−113 | 0.285179 |
| Ier3 | 9.36E−113 | 0.284854 |
| Ski | 5.58E−109 | 0.280158 |
| Fam105a | 3.92E−108 | 0.279092 |
| Car4 | 8.33E−108 | 0.278679 |
| Krtcap2 | 2.87E−107 | 0.277999 |
| Bcl2a1d | 5.45E−106 | 0.276373 |

Table 2 shows the human orthologs for murine genes used for T-SiglecF^{high} neutrophil gene signature.

TABLE 2
Human orthologs for murine genes used for T-SiglecF^{high} neutrophil gene signature.

| 0 | human_ortholog | human_ortholog_present_in_microarray_data |
|---|---|---|
| Spp1 | [‘SPP1’] | [‘SPP1’] |
| Ltc4s | [‘LTC4S’] | [‘LTC4S’] |
| Gpr171 | [‘GPR171’] | [‘GPR171’] |
| Ralgds | [‘RALGDS’, ‘RGL1’, ‘RGL4’] | [‘RALGDS’, ‘RGL1’] |
| Rab15 | [‘RAB15’] | [‘RAB15’] |
| Scimp | [‘SCIMP’] | [ ] |
| Samm50 | [‘SAMM50’] | [‘SAMM50’] |
| Il1a | [‘IL1A’] | [‘IL1A’] |
| Adamdec1 | [‘ADAMDEC1’] | [‘ADAMDEC1’] |
| Mreg | [‘MREG’] | [‘MREG’] |
| Nme2 | [‘NME1’, ‘NME1-NME2’, ‘NME2’, | [‘NME1’, ‘NME3’, ‘NME4’] |
| | ‘NME2P1’, ‘NME3’, ‘NME4’] | |
| Cox16 | [‘COX16’] | [‘COX16’] |
| Cxcl14 | [‘CXCL14’] | [‘CXCL14’] |
| Tmem147 | [‘TMEM147’] | [‘TMEM147’] |
| Lrp1 | [‘LRP1’, ‘LRP1B’, ‘LRP2’, | [‘LRP1’, ‘LRP1B’, ‘LRP2’, |
| | ‘LRP5’, ‘LRP6’] | ‘LRP5’, ‘LRP6’] |
| H1f0 | [‘H1F0’] | [‘H1F0’] |
| Tnfrsf13b | [‘TNFRSF13B’] | [‘TNFRSF13B’] |
| Nme1 | [‘NME1’, ‘NME1-NME2’, ‘NME2’, | [‘NME1’, ‘NME3’, ‘NME4’] |
| | ‘NME3’, ‘NME4’] | |
| Mif | [‘MIF’] | [‘MIF’] |
| Ranbp1 | [‘RANBP1’] | [‘RANBP1’] |
| Hexb | [‘HEχB’] | [‘HEXB’] |
| Lgmn | [‘LGMN’] | [‘LGMN’] |
| Ms4a6d | [‘MS4A6A’, ‘MS4A6E’] | [‘MS4A6A’] |
| P2ry14 | [‘P2RY14’] | [‘P2RY14’] |
| Acp5 | [‘ACP5’] | [‘ACP5’] |
| Gm4076 | [‘MT-ND5’] | [ ] |
| S1pr2 | [‘S1PR2’] | [‘S1PR2’] |
| Fundc2 | [‘FUNDC2’] | [ ] |
| Hspa9 | [‘HSPA13’, ‘HSPA14’, ‘HSPA1A’, | [‘HSPA13’, ‘HSPA14’, ‘HSPA1A’, |
| | ‘HSPA1B’, ‘HSPA1L’, ‘HSPA2’, ‘HSPA4’, | ‘HSPA1L’, ‘HSPA2’, ‘HSPA4’, ‘HSPA4L’, |
| | ‘HSPA4L’, ‘HSPA5’, ‘HSPA6’, ‘HSPA7’, | ‘HSPA5’, ‘HSPA6’, ‘HSPA8’, ‘HSPA9’, |
| | ‘HSPA8’, ‘HSPA9’, ‘HSPH1’, ‘HYOU1’] | ‘HSPH1’, ‘HYOU1’] |
| Ffar2 | [‘FFAR2’] | [‘FFAR2’] |
| Hsd17b12 | [‘HSD17B12’] | [‘HSD17B12’] |
| Dbi | [‘DBI’] | [‘DBI’] |
| Car4 | [‘CA4’] | [‘CA4’] |
| Elk3 | [‘ELK3’] | [‘ELK3’] |
| Rhoq | [‘RHOJ’, ‘RHOQ’] | [‘RHOQ’] |
| Mat2a | [‘MAT1A’, ‘MAT2A’] | [‘MAT1A’, ‘MAT2A’] |
| Ddt | [‘DDT’, ‘DDTL’] | [‘DDT’] |
| E2f4 | [‘E2F4’] | [‘E2F4’] |
| Polr2m | [‘GCOM2’, ‘POLR2M’] | [ ] |
| Rcc2 | [‘RCC2’] | [ ] |
| Prss2 | [‘PRSS1’, ‘PRSS2’, ‘PRSS3’] | [‘PRSS1’, ‘PRSS2’, ‘PRSS3’] |
| Ctsc | [‘CTSC’] | [‘CTSC’] |
| Tcof1 | [‘TCOF1’] | [‘TCOF1’] |
| Erdr1 | N/A | [ ] |
| Trio | [‘KALRN’, ‘TRIO’] | [‘KALRN’, ‘TRIO’] |
| Pebp1 | [‘PEBP1’] | [‘PEBP’] |
| Odc1 | [‘ODC1’] | [‘ODC1’] |
| Slc23a2 | [‘SLC23A1’, ‘SLC23A2’] | [‘SLC23A2’] |
| Pald1 | [‘PALD1’] | [ ] |
| Abi3 | [‘ABI3’] | [ ] |
| Rgs10 | [‘RGS10’] | [‘RGS10’] |
| Siglecf | [‘CD33’, ‘SIGLEC14’, ‘SIGLEC5’, | [‘CD33’, ‘SIGLEC5’, ‘SIGLEC6’, |
| | ‘SIGLEC6’, ‘SIGLEC8’] | ‘SIGLEC8’] |
| Src | [‘FGR’, ‘FYN’, ‘SRC’, ‘YES1’] | [‘FGR’, ‘FYN’, ‘SRC’, |
| | | ‘YES1’] |
| Snx8 | [‘MIR6836’, ‘SNX8’] | [ ] |
| Ccdc86 | [‘CCDC86’] | [‘CCDC86’] |
| Nucks1 | [‘NUCKS1’] | [‘NUCKS1’] |
| Plagl2 | [‘PLAGL2’] | [‘PLAGL2’] |
| Gm13092 | N/A | [ ] |
| Nt5e | [‘NT5E’] | [‘NT5E’] |
| G3bp1 | [‘G3BP1’] | [‘G3BP1’] |
| Ddx21 | [‘DDX21', ‘DDX50’] | [‘DDX21’, ‘DDX50’] |
| Ptgs1 | [‘PTGS1’, ‘PTGS2’] | [‘PTGS1’, ‘PTGS2’] |
| Sptssa | [‘SPTSSA’] | [ ] |
| Ormdl1 | [‘ORMDL1’, ‘ORMDL2’, ‘ORMDL3’] | [‘ORMDL2’] |
| Cish | [‘CISH’] | [‘CISH’] |
| B4galt1 | [‘B4GALT1’, ‘B4GALT2’] | [‘B4GALT1’, ‘B4GALT2’] |
| Mcoln1 | [‘MCOLN1’] | [‘MCOLN1’] |
| Igfbp6 | [‘IGFBP6’] | [‘IGFBP6’] |
| Nop10 | [‘NOP10’] | [‘NOP10’] |
| U2af1l4 | [‘AD000671.2’, ‘IGFLR1’, ‘U2AF1’, | [‘U2AF1’] |
| | ‘U2AF1L4’, ‘U2AF1L5’] | |
| Mrpl52 | [‘MRPL52’] | [‘MRPL52’] |
| Zranb2 | [‘ZRANB2’] | [ ] |
| Nckap5l | [‘NCKAP5L’] | [ ] |
| Ilf3 | [‘ILF3’, ‘STRBP’] | [‘ILF3’] |
| Nhp2 | [‘NHP2’] | [‘NHP2’] |
| Ccl3 | [‘CCL18’, ‘CCL3’, ‘CCL3L3’, | [‘CCL18’, ‘CCL4’] |
| | ‘CCL4’, ‘CCL4L2’] | |
| Gns | [‘GNS’] | [‘GNS’] |
| Rnf187 | [‘RNF187’] | [‘RNF187’] |
| mt-Nd2 | [‘MT-ND2’] | [ ] |
| Polr2f | [‘POLR2F’] | [‘POLR2F’] |
| Pdcd1lg2 | [‘PDCD1LG2’] | [‘PDCD1LG2’] |
| Ubash3b | [‘UBASH3B’] | [ ] |
| mt-Nd1 | [‘MT-ND1’] | [ ] |
| Ccdc115 | [‘CCDC115’] | [ ] |
| Npm1 | [‘NPM1’] | [‘NPM1’] |
| Krtcap2 | [‘KRTCAP2’] | [ ] |
| Aebp2 | [‘AEBP2’] | [ ] |
| Plbd2 | [‘PLBD2’] | [ ] |
| Snrpd2 | [‘SNRPD2’] | [‘SNRPD2’] |
| Dtx4 | [‘DTX1’, ‘DTX4’] | [‘DTX4’] |
| Clec4n | [‘CLEC6A’] | [ ] |
| Eif3b | [‘EIF3B’] | [‘EIF3B’] |
| Hipk3 | [‘HIPK1’, ‘HIPK2’, ‘HIPK3’] | [‘HIPK1’, ‘HIPK2’, ‘HIPK3’] |
| Fam20c | [‘FAM20C’] | [ ] |
| AA467197 | [‘C15orf48’] | [ ] |
| Tmem65 | [‘TMEM65’] | [ ] |
| Banf1 | [‘BANF1’] | [‘BANF1’] |
| S100a1 | [‘S100A1’] | [‘S100A1’] |
| Rab18 | [‘RAB18’] | [ ] |
| Col15a1 | [‘COL15A1’, ‘COL18A1’] | [‘COL15A1’, ‘COL18A1’] |
| Gstm1 | [‘GSTM1’, ‘GSTM2’, ‘GSTM3’, | [‘GSTM1’, ‘GSTM2’, ‘GSTM3’, |
| | ‘GSTM4’, ‘GSTM5’] | ‘GSTM4’, ‘GSTM5’] |
| Srsf9 | [‘SRSF9’] | [‘SRSF9’] |
| Itm2c | [‘ITM2C’] | [‘ITM2C’] |
| Bhlhe40 | [‘BHLHE40’] | [‘BHLHE40’] |
| Tnfrsf26 | [‘LOC254896’, ‘TNFRSF10A’, ‘TNFRSF10B’, | [‘LOC254896’, ‘TNFRSF10B’, |
| | ‘TNFRSF10D’] | ‘TNFRSF10D’] |
| Atp5o | [‘AP000311.1’, ‘ATP5O’] | [‘ATP5O’] |
| Fmnl3 | [‘FMNL1’, ‘FMNL2’, ‘FMNL3’] | [‘FMNL1’] |
| mt-Nd5 | [‘MT-ND5’] | [ ] |
| 1810011H11Rik | [‘C10orf128’] | [ ] |
| Creb5 | [‘CREB5’] | [‘CREB5’] |
| Stx3 | [‘STX3’] | [‘STX3’] |
| Cstb | [‘CSTB’] | [‘CSTB’] |
| mt-Nd3 | [‘MT-ND3’] | [ ] |
| Hmga1-rs1 | [‘HMGA1’, ‘HMGA2’] | [‘HMGA1’, ‘HMGA2’] |
| Ran | [‘RAN’] | [‘RAN’] |
| Cd81 | [‘CD81’] | [‘CD81’] |
| Mt1 | [‘MT1A’, ‘MT1B’, ‘MT1DP’, | [‘MT1E’, ‘MT1F’, ‘MT1G’, |
| | ‘MT1E’, ‘MT1F’, ‘MT1G’, | ‘MT1H’, ‘MT1M’, ‘MT1X’, |
| | ‘MT1H’, ‘MT1L’, ‘MT1M’, | ‘MT2A’] |
| | ‘MT1X, ‘MT2A’] | |
| Cmas | [‘CMAS’] | [‘CMAS’] |
| Chil3 | [‘CHI3L1’, ‘CHI3L2’, ‘CHIA’, | [‘CHI3L1’, ‘CHI3L2’, ‘CHIA’, |
| | ‘CHIT1’] | ‘CHIT1’] |
| Igsf8 | [‘IGSF8’] | [ ] |
| Ctnna1 | [‘CTNNA1’, ‘CTNNA2’, ‘CTNNA3’] | [‘CTNNA1’, ‘CTNNA2’, ‘CTNNA3’] |
| Eprs | [‘EPRS’] | [‘EPRS’] |
| Ube2e1 | [‘UBE2E1’, ‘UBE2E2’, ‘UBE2E3’] | [‘UBE2E1’, ‘UBE2E3’] |
| Snx2 | [‘SNX2’] | [‘SNX2’] |
| Fam110a | [‘FAM110A’] | [ ] |
| Xpo1 | [‘χPO1’] | [‘χPO1’] |
| D15Ertd621e | N/A | [ ] |
| Rpsa | [‘RPSA’, ‘RPSAP58’] | [‘RPSA’] |
| Il1rl2 | [‘IL1RL2’] | [‘IL1RL2’] |
| Pole4 | [‘POLE4’] | [ ] |
| Camkk1 | [‘CAMKK1’, ‘CAMKK2’] | [‘CAMKK2’] |
| Tcirg1 | [‘ATP6V0A1’, ‘ATP6V0A2’, ‘ATP6V0A4’, | [‘ATP6V0A1’, ‘ATP6V0A2’, ‘ATP6V0A4’, |
| | ‘TCIRG1’] | ‘TCIRG1’] |
| Arl1 | [‘ARL1’] | [‘ARL1’] |
| Pld3 | [‘PLD3’] | [‘PLD3’] |
| Dpp9 | [‘DPP8’, ‘DPP9’] | [‘DPP8’] |
| Colgalt1 | [‘CERCAM’, ‘COLGALT1’, ‘COLGALT2’] | [ ] |
| Slc39a1 | [‘SLC39A1’] | [‘SLC39A1’] |
| Gm11410 | N/A | [ ] |
| Nudt3 | [‘NUDT3’] | [‘NUDT3’] |
| Ddb1 | [‘DDB1’] | [‘DDB1’] |
| Secisbp21 | [‘SECISBP2L’] | [‘SECISBP2L’] |
| Gusb | [‘GUSB’, ‘GUSBP1’, ‘GUSBP11’] | [‘GUSB’] |
| C330007P06Rik | [‘CXorf56’] | [‘CXorf56’] |
| Irak2 | [‘IRAK2’] | [ ] |
| Basp1 | [‘BASP1’] | [‘BASP1’] |
| Hmga1 | [‘HMGA1’, ‘HMGA2’] | [‘HMGA1’, ‘HMGA2’] |
| Utp18 | [‘UTP18’] | [‘UTP18’] |
| Fam105a | [‘FAM105A’] | [‘FAM105A’] |
| mt-Atp8 | [‘MT-ATP8’] | [ ] |
| Ostc | [‘OSTC’] | [ ] |
| Rbmxl1 | [‘RBMX’, ‘RBMXL1’, ‘RBMXL2’, | [‘RBMX’, ‘RBMXL2’] |
| | ‘RBMY1A1’, ‘RBMY1B’, ‘RBMY1D’, | |
| | ‘RBMY1E’, ‘RBMY1F’, ‘RBMY1J’] | |
| Mrps33 | [‘MRPS33’] | [‘MRPS33’] |
| Nceh1 | [‘NCEH1’] | [ ] |
| Nsun2 | [‘NSUN2’] | [ ] |
| Ski | [‘SKI’] | [‘SKI’] |
| Uqcrb | [‘UQCRB’] | [‘UQCRB’] |
| Med12l | [‘MED12’, ‘MED12L’] | [‘MED12’] |
| Rpl36al | [‘RPL36A’, ‘RPL36A-HNRNPH2’, ‘RPL36AL’] | [‘RPL36A’, ‘RPL36AL’] |
| Zfp91 | [‘ZFP91’, ‘ZFP91-CNTF’] | [ ] |
| Phb2 | [‘PHB2’] | [‘PHB2’] |
| Stip1 | [‘STIP1’] | [‘STIP1’] |
| Maff | [‘MAFF’] | [‘MAFF’] |
| Sf3b3 | [‘SF3B3’] | [‘SF3B3’] |
| Mapkapk3 | [‘MAPKAPK2’, ‘MAPKAPK3’] | [‘MAPKAPK2’, ‘MAPKAPK3’] |
| ID2 | [‘ID2’] | [‘ID2’] |
| Ggta1 | [‘GGTA1P’, ‘GLT6D1’] | [ ] |
| Gm10275 | N/A | [ ] |
| Ppia | [‘LOC105371242’, ‘PPIA’, ‘PPIAL4A’, | [‘PPIA’, ‘PPIAL4A’, ‘PPIF’] |
| | ‘PPIAL4C’, ‘PPIAL4D’, ‘PPIAL4E’, | |
| | ‘PPIAL4F’, ‘PPIAL4G’, ‘PPIF’] | |
| Gm11407 | N/A | [ ] |
| Arfgcf2 | [‘ARFGEF1’, ‘ARFGEF2’] | [‘ARFGEF1’, ‘ARFGEF2’] |
| Rps19 | [‘RPS19’] | [‘RPS19’] |
| Rad23b | [‘RAD23B’] | [‘RAD23B’] |
| Ntpcr | [‘NTPCR’] | [ ] |
| Nutf2-ps1 | [‘NUTF2’] | [‘NUTF2’] |
| Dgcr2 | [‘DGCR2’] | [‘DGCR2’] |
| Pmf1 | [‘PMF1’, ‘PMF1-BGLAP’] | [‘PMF1’] |
| Rps17 | [‘RPS17’] | [‘RPS17’] |
| Arl8b | [‘ARL8A’, ‘ARL8B’] | [‘ARL8B’] |
| Rnps1 | [‘RNPS1’] | [‘RNPS1’] |
| Gm9825 | [‘RNPS1’] | [‘RNPS1’] |
| Isy1 | [‘ISY1’, ‘ISY1-RAB43’] | [ ] |
| Ptbp1 | [‘PTBP1’, ‘PTBP2’, ‘PTBP3’] | [‘PTBP’, ‘PTBP2’] |
| Gsto1 | [‘GSTO1’, ‘GSTO2’] | [‘GSTO1’] |
| Runx1 | [‘LOC100506403’, ‘RUNX1’] | [‘RUNX1’] |
| Upp1 | [‘UPP1’] | [‘UPP1’] |
| Pfdn6 | [‘PFDN6’] | [‘PFDN6’] |
| Snrpe | [‘SNRPE’] | [‘SNRPE’] |
| Smn1 | [‘SMN1’, ‘SMN2’] | [ ] |
| Cdkn1a | [‘CDKN1A’] | [‘CDKN1A’] |
| Hilpda | [‘HILPDA’] | [ ] |
| Cct2 | [‘CCT2’] | [‘CCT2’] |
| mt-Cytb | [‘MT-CYB’] | [ ] |
| Ddx50 | [‘DDX21’, ‘DDX50’] | [‘DDX21’, ‘DDX50’] |
| Uck2 | [‘MIR3658’, ‘UCK1’, ‘UCK2’] | [‘UCK2’] |
| Vegfa | [‘VEGFA’] | [‘VEGFA’] |
| Pigt | [‘PIGT’] | [‘PIGT’] |
| Itgb1 | [‘ITGB1’, ‘ITGB2’, ‘ITGB5’, | [‘ITGB1’, ‘ITGB2’, ‘ITGB5’, |
| | ‘ITGB6’, ‘ITGB7’] | ‘ITGB6’, ‘ITGB7’] |
| Zrsr2 | [‘ZRSR1’, ‘ZRSR2’] | [‘ZRSR2’] |
| Tbca | [‘TBCA’] | [‘TBCA’] |
| Emg1 | [‘EMG1’] | [‘EMG1’] |
| Eif3g | [‘EIF3G’] | [‘EIF3G’] |
| Ctsb | [‘CTSB’] | [‘CTSB’] |
| Emc1 | [‘EMC1’] | [ ] |
| Scd2 | [‘SCD’, ‘SCD5’] | [‘SCD’, ‘SCD5’] |
| Igf2r | [‘IGF2R’] | [‘IGF2R’] |
| Tnf | [‘TNF’] | [‘TNF’] |
| Papd5 | [‘PAPD5’] | [ ] |
| Ssbp4 | [‘SSBP2’, ‘SSBP3’, ‘SSBP4’] | [‘SSBP2’, ‘SSBP3’] |
| Bms1 | [‘BMS1’] | [‘BMS1’] |
| Nek6 | [‘NEK6’, ‘NEK7’] | [‘NEK7’] |
| Csf1 | [‘CSF1’] | [‘CSF1’] |
| Gm3244 | [‘NDUFB4’] | [‘NDUFB4’] |
| Lemd2 | [‘LEMD2’] | [ ] |
| Nap1l4 | [‘NAP1L4’] | [‘NAP1L4’] |
| Hivep2 | [‘HIVEP2’, ‘HIVEP3’] | [‘HIVEP2’, ‘HIVEP3’] |
| Atp6v1c1 | [‘ATP6V1C1’] | [‘ATP6V1C1’] |
| Gps1 | [‘GPS1’] | [‘GPS1’] |
| Ndufb4 | [‘NDUFB4’] | [‘NDUFB4’] |
| Psmc4 | [‘PSMC4’] | [‘PSMC4’] |
| Eif4e | [‘EIF4E’, ‘EIF4E1B’] | [‘EIF4E’] |
| Adgrg3 | [‘ADGRG3’] | [ ] |
| Rps18 | [‘RPS18’] | [‘RPS18’] |
| Ndufb6 | [‘NDUFB6’] | [‘NDUFB6’] |
| Nol7 | [‘NOL7’] | [‘NOL7’] |
| Pdia6 | [‘PDIA6’] | [‘PDIA6’] |
| Adgre1 | [‘ADGRE1’] | [ ] |
| Psmb6 | [‘PSMB6’] | [‘PSMB6’] |
| Cd63 | [‘CD63’] | [‘CD63’] |
| Trim28 | [‘TRIM28’] | [‘TRIM28’] |
| Alyref | [‘ALYREF’] | [ ] |
| Taf3 | [‘TAF3’] | [ ] |
| Cct3 | [‘CCT3’] | [‘CCT3’] |
| Emc10 | [‘EMC10’] | [ ] |
| Tcp1 | [‘TCP1’] | [‘TCP1’] |
| Slc38a1 | [‘SLC38A1’] | [‘SLC38A1’] |
| Tbk1 | [‘IKBKE’, ‘TBK1’] | [‘IKBKE’, ‘TBK1’] |
| Cct8 | [‘CCT8’] | [‘CCT8’] |
| Ak6 | [‘AK6’, ‘TAF9’, ‘TAF9B’] | [‘TAF9’, ‘TAF9B’] |
| Cox7b | [‘COX7B’, ‘COX7B2’] | [‘COX7B’] |
| Slc31a2 | [‘SLC31A2’] | [‘SLC31A2’] |
| 2610001J05Rik | [‘SMIM30’] | [ ] |
| Cd63-ps | N/A | [ ] |
| Glrx5 | [‘GLRX5’] | [‘GLRX5’] |
| Mgat4b | [‘MGAT4A’, ‘MGAT4B’] | [‘MGAT4A’, ‘MGAT4B’] |
| Cycs | [‘CYCS’] | [‘CYCS’] |
| Gm10263 | [‘RPS28’] | [‘RPS28’] |
| Bcl2a1b | [‘BCL2A1’] | [‘BCL2A1’] |
| Gm5835 | N/A | [ ] |
| Dad1 | [‘DAD1’] | [‘DAD1’] |
| Nudcd3 | [‘NUDCD3’] | [‘NUDCD3’] |
| Rps28 | [‘RPS28’] | [‘RPS28’] |
| Aprt | [‘APRT’] | [‘APRT’] |
| Gabpa | [‘GABPA’] | [‘GABPA’] |
| Tsg101 | [‘TSG101’] | [‘TSG101’] |
| Frrs1 | [‘FRRS1’] | [ ] |
| Gm14586 | N/A | [ ] |
| Tpi1 | [‘TPI1’] | [‘TPI1’] |
| Jak3 | [‘JAK3’] | [‘JAK3’] |
| Eea1 | [‘EEA1’] | [‘EEA1’] |
| Gngt2 | [‘GNGT2’] | [ ] |
| Peli2 | [‘PELI1’, ‘PELI2’, ‘PELI3’] | [‘PELI1’, ‘PELI2’] |
| Trmt112 | [‘TRMT112’] | [‘TRMT112’] |
| Rpl10a-ps1 | N/A | [ ] |
| Idh3b | [‘IDH3B’] | [‘IDH3B’] |
| Swi5 | [‘SWI5’] | [ ] |
| Anxa5 | [‘ANXA5’] | [‘ANχA5’] |
| Nup153 | [‘NPAP1’, ‘NUP153’] | [‘NUP153’] |
| Gm10053 | [‘CYCS’] | [‘CYCS’] |
| Rbbp5 | [‘RBBP5’] | [‘RBBP5’] |
| Ssh1 | [‘SSH1’, ‘SSH2’] | [‘SSH1’] |
| Eif4g1 | [‘EIF4G1’, ‘EIF4G3’] | [‘EIF4G1’, ‘EIF4G3’] |
| Tmem230 | [‘TMEM230’] | [ ] |
| Srsf7 | [‘SRSF7’] | [‘SRSF7’] |
| Btbd19 | [‘BTBD19’] | [ ] |
| 3110082I17Rik | [‘C7orf50’] | [ ] |
| Irf3 | [‘IRF3’] | [‘IRF3’] |
| Fam178a | N/A | [ ] |
| Ero1l | [‘ERO1A’, ‘ERO1B’] | [ ] |
| mt-Atp6 | [‘MT-ATP6’] | [ ] |
| Rps27l | [‘RPS27’, ‘RPS27L’] | [‘RPS27’, ‘RPS27L’] |
| Bcl2a1a | [‘BCL2A1’] | [‘BCL2A1’] |
| Usp2 | [‘USP2’] | [‘USP2’] |
| Runx3 | [‘RUNX3’] | [‘RUNX3’] |
| Clec5a | [‘CLEC5A’] | [‘CLEC5A’] |
| Rpl10a | [‘RPL10A’] | [‘RPL10A’] |
| Tmbim1 | [‘FAIM2’, ‘MIR6513’, ‘TMBIM1’] | [‘FAIM2’, ‘TMBIM1’] |
| Wdr83os | [‘WDR83OS’] | [ ] |
| Znrd1 | [‘ZNRD1’] | [ ] |
| Srebf2 | [‘SREBF1’, ‘SREBF2’] | [‘SREBF1’, ‘SREBF2’] |
| Rpl36a | [‘RPL36A’, ‘RPL36A-HNRNPH2’, ‘RPL36AL’] | [‘RPL36A’, ‘RPL36AL’] |
| Snrpg | [‘SNRPG’] | [‘SNRPG’] |
| Elovl5 | [‘ELOVL5’] | [‘ELOVL5’] |
| Ssr4 | [‘SSR4’] | [‘SSR4’] |
| Asna1 | [‘ASNA1’] | [‘ASNA1’] |
| Mrps14 | [‘MRPS14’] | [‘MRPS14’] |
| Gga3 | [‘GGA1’, ‘GGA3’] | [‘GGA1’, ‘GGA3’] |
| Gpaa1 | [‘GPAA1’] | [‘GPAA1’] |
| Bag1 | [‘BAG1’] | [‘BAG1’] |
| Tbcb | [‘TBCB’] | [‘TBCB’] |
| Rps8-ps1 | N/A | [ ] |
| Amdhd2 | [‘AC093525.1’, ‘AMDHD2’] | [‘AMDHD2’] |
| Gm16210 | N/A | [ ] |
| Cenpb | [‘CENPB’] | [‘CENPB’] |
| Vimp | N/A | [ ] |
| Btf314 | [‘BTF3’, ‘BTF3L4’] | [‘BTF3’] |

What is claimed is:

1. (canceled)

2. A method for treating cancer, the method comprising:
identifying a population of SiglecF^{high} cells in a patient; and
administering an agent to the patient that inhibits the activity, level, and/or migration of said population of SiglecF^{high} cells.

3. The method of claim 2, wherein the cancer is lung cancer.

4. The method of claim 3, wherein the lung cancer is non-small cell lung cancer.

5. The method of claim 4, wherein the is non-small cell lung cancer is KRAS⁺ non-small cell lung cancer.

6. The method of claim 3, wherein the lung cancer is selected from the group consisting of: small cell lung cancer, small cell carcinoma, combined small cell carcinoma, lung carcinoid tumor, adenocarcinoma, squamous cell carcinoma, a solid tumor cancer, or large cell carcinoma.

7. (canceled)

8. The method of claim 2, wherein the agent is selected from the group consisting of: a small molecule, an inhibitory nucleic acid, an antibody or antigen-binding fragment thereof, or antibody reagent, an inhibitory polypeptide, an antisense oligonucleotide, an immunotherapy, a nanoparticle, and a polymer.

9.-15. (canceled)

16. The method of claim 2, wherein the inhibiting the level kills the SiglecF^{high} cell.

17. The method of claim 2, wherein the inhibiting the level inhibits the rate at which the SiglecF^{high} cell is induced.

18. The method of claim 2, wherein the inhibiting the activity puts the cell into anergy.

19. The method of claim 2, wherein the inhibiting the activity disrupts the functional interaction of the SiglecF^{high} cell and a tumor cell, and/or disrupts the functional interaction of the SilecF^{high} cell and a tumor microenvironment.

20. (canceled)

21. The method of claim 2, wherein the inhibiting the activity disrupts the tumor-promoting function of a SiglecF^{high} cell.

22. The method of claim 2, wherein the inhibiting the migration disrupts the physical interaction of the SiglecF^{high} cell and a tumor cell, and/or disrupts the physical interaction of the SiglecF^{high} cell and a tumor microenvironment.

23. (canceled)

24. The method of claim 2, wherein the inhibiting the migration disrupts the movement of the SiglecF^{high} cell towards a tumor cell, and/or disrupts the movement of the SiglecF^{high} cell towards a tumor microenvironment.

25. (canceled)

26. The method of claim 2, wherein the inhibiting the migration inhibits the tropism of the SiglecF^{high} cell.

27. The method of claim 2, wherein the identifying a population of SiglecF^{high} cells comprises: assessing the gene profile of a sample obtained from said patient; and comparing it to the gene profile of SiglecF^{high} cells.

28. The method of claim 27, wherein the sample is obtained from a biopsy of a lung, lymph node, or bronchoalveolar lavage.

29. (canceled)

30. (canceled)

31. The method of claim 2, further administering a second therapeutic.

32. The method of claim 31, wherein the second therapeutic is a chemotherapeutic, an anti-tumor agent, radiation, or surgery.

33.-39. (canceled)

40. A method for treating cancer, the method comprising:
receiving the results of an assay that identifies a patient as having a population of SiglecF^{high} cells; and
administering an agent to the patient that inhibits the activity, level, and/or migration of said population of SiglecF^{high} cells.

41. A composition comprising an agent that inhibits the activity, level, and/or migration of said population of SiglecF^{high} cells.
